SEQUENCE LISTING (32 sequences)

SEQ ID NO: 1 - DNA, 22 bp, synthetic construct
  misc_feature 1..22: note = Description of sequence: Primer: Fw-sequence; target gene: BMP2
  source 1..22: mol_type = other DNA
ctacatgctagacctgtatcgc

SEQ ID NO: 2 - DNA, 21 bp, synthetic construct
  misc_feature 1..21: note = Description of sequence: Primer: Rv-sequence; target gene: BMP2
  source 1..21: mol_type = other DNA
cccactcgtttctggtagttc

SEQ ID NO: 3 - DNA, 21 bp, synthetic construct
  misc_feature 1..21: note = Description of sequence: Primer: Fw-sequence; target gene: RUNX2
  source 1..21: mol_type = other DNA
ttcaccttgaccataaccgtc

SEQ ID NO: 4 - DNA, 21 bp, synthetic construct
  misc_feature 1..21: note = Description of sequence: Primer: Rv-sequence; target gene: RUNX2
  source 1..21: mol_type = other DNA
ggcggtcagagaacaaactag

SEQ ID NO: 5 - DNA, 21 bp, synthetic construct
  misc_feature 1..21: note = Description of sequence: Primer: Fw-sequence; target gene: SP7
  source 1..21: mol_type = other DNA
ctccctctcccttttctctct

SEQ ID NO: 6 - DNA, 21 bp, synthetic construct
  misc_feature 1..21: note = Description of sequence: Primer: Rv-sequence; target gene: SP7
  source 1..21: mol_type = other DNA
ggagccatagtgaacttcctc

SEQ ID NO: 7 - DNA, 22 bp, synthetic construct
  misc_feature 1..22: note = Description of sequence: Primer: Fw-sequence; target gene: ALPL
  source 1..22: mol_type = other DNA
gatgtggagtatgagagtgacg

SEQ ID NO: 8 - DNA, 19 bp, synthetic construct
  misc_feature 1..19: note = Description of sequence: Primer: Rv-sequence; target gene: ALPL
  source 1..19: mol_type = other DNA
ggtcaagggtcaggagttc

SEQ ID NO: 9 - DNA, 22 bp, synthetic construct
  misc_feature 1..22: note = Description of sequence: Primer: Fw-sequence; target gene: MKI67
  source 1..22: mol_type = other DNA
aaaagaattgaacctgcggaag

SEQ ID NO: 10 - DNA, 22 bp, synthetic construct
  misc_feature 1..22: note = Description of sequence: Primer: Rv-sequence; target gene: MKI67
  source 1..22: mol_type = other DNA
agtcttattttggcgtctggag

SEQ ID NO: 11 - DNA, 19 bp, synthetic construct
  misc_feature 1..19: note = Description of sequence: Primer: Fw-sequence; target gene: Caspase3
  source 1..19: mol_type = other DNA
actggactgtggcattgag

SEQ ID NO: 12 - DNA, 21 bp, synthetic construct
  misc_feature 1..21: note = Description of sequence: Primer: Rv-sequence; target gene: Caspase3
  source 1..21: mol_type = other DNA
gagccatcctttgaatttcgc

SEQ ID NO: 13 - DNA, 22 bp, synthetic construct
  misc_feature 1..22: note = Description of sequence: Primer: Fw-sequence; target gene: Caspase9
  source 1..22: mol_type = other DNA
ccaaccctagaaaaccttaccc

SEQ ID NO: 14 - DNA, 21 bp, synthetic construct
  misc_feature 1..21: note = Description of sequence: Primer: Rv-sequence; target gene: Caspase9
  source 1..21: mol_type = other DNA
tctgcatttcccctcaaactc

SEQ ID NO: 15 - DNA, 20 bp, synthetic construct
  misc_feature 1..20: note = Description of sequence: Primer: Fw-sequence; target gene: IL-6
  source 1..20: mol_type = other DNA
ccactcacctcttcagaacg

SEQ ID NO: 16 - DNA, 22 bp, synthetic construct
  misc_feature 1..22: note = Description of sequence: Primer: Rv-sequence; target gene: IL-6
  source 1..22: mol_type = other DNA
catctttggaaggttcaggttg

SEQ ID NO: 17 - DNA, 19 bp, synthetic construct
  misc_feature 1..19: note = Description of sequence: Primer: Fw-sequence; target gene: NOS2
  source 1..19: mol_type = other DNA
gtttgaccagaggacccag

SEQ ID NO: 18 - DNA, 21 bp, synthetic construct
  misc_feature 1..21: note = Description of sequence: Primer: Rv-sequence; target gene: NOS2
  source 1..21: mol_type = other DNA
atctcctttgttaccgcttcc

SEQ ID NO: 19 - DNA, 21 bp, synthetic construct
  misc_feature 1..21: note = Description of sequence: Primer: Fw-sequence; target gene: COL1A1
  source 1..21: mol_type = other DNA
cccctggaaagaatggagatg

SEQ ID NO: 20 - DNA, 21 bp, synthetic construct
  misc_feature 1..21: note = Description of sequence: Primer: Rv-sequence; target gene: COL1A1
  source 1..21: mol_type = other DNA
tccaaaccactgaaacctctg

SEQ ID NO: 21 - DNA, 20 bp, synthetic construct
  misc_feature 1..20: note = Description of sequence: Primer: Fw-sequence; target gene: COL1A2
  source 1..20: mol_type = other DNA
aggacaagaaacacgtctgg

SEQ ID NO: 22 - DNA, 22 bp, synthetic construct
  misc_feature 1..22: note = Description of sequence: Primer: Rv-sequence; target gene: COL1A2
  source 1..22: mol_type = other DNA
ggtgatgttctgagaggcatag

SEQ ID NO: 23 - DNA, 21 bp, synthetic construct
  misc_feature 1..21: note = Description of sequence: Primer: Fw-sequence; target gene: COL3A1
  source 1..21: mol_type = other DNA
aagtcaaggagaaagtggtcg

SEQ ID NO: 24 - DNA, 22 bp, synthetic construct
  misc_feature 1..22: note = Description of sequence: Primer: Rv-sequence; target gene: COL3A1
  source 1..22: mol_type = other DNA
ctcgttctccattcttaccagg

SEQ ID NO: 25 - DNA, 21 bp, synthetic construct
  misc_feature 1..21: note = Description of sequence: Primer: Fw-sequence; target gene: COL4A3
  source 1..21: mol_type = other DNA
cccacaggcatagaaggattc

SEQ ID NO: 26 - DNA, 21 bp, synthetic construct
  misc_feature 1..21: note = Description of sequence: Primer: Rv-sequence; target gene: COL4A3
  source 1..21: mol_type = other DNA
gcctattacatgactccctgg

SEQ ID NO: 27 - DNA, 21 bp, synthetic construct
  misc_feature 1..21: note = Description of sequence: Primer: Fw-sequence; target gene: COL5A1
  source 1..21: mol_type = other DNA
cggaaccttgacgagaactac

SEQ ID NO: 28 - DNA, 20 bp, synthetic construct
  misc_feature 1..20: note = Description of sequence: Primer: Rv-sequence; target gene: COL5A1
  source 1..20: mol_type = other DNA
tctcccttttggcctttctc

SEQ ID NO: 29 - DNA, 20 bp, synthetic construct
  misc_feature 1..20: note = Description of sequence: Primer: Fw-sequence; target gene: TGFB1
  source 1..20: mol_type = other DNA
gcctttcctgcttctcatgg

SEQ ID NO: 30 - DNA, 21 bp, synthetic construct
  misc_feature 1..21: note = Description of sequence: Primer: Rv-sequence; target gene: TGFB1
  source 1..21: mol_type = other DNA
tccttgcggaagtcaatgtac

SEQ ID NO: 31 - DNA, 20 bp, synthetic construct
  misc_feature 1..20: note = Description of sequence: Primer: Fw-sequence; target gene: hPPIA
  source 1..20: mol_type = other DNA
gccgaggaaaaccgtgtact

SEQ ID NO: 32 - DNA, 22 bp, synthetic construct
  misc_feature 1..22: note = Description of sequence: Primer: Rv-sequence; target gene: hPPIA
  source 1..22: mol_type = other DNA
ctgcaaacagctcaaaggagac

(19) United States
(12) Patent Application Publication
(10) Pub. No.: US 2026/0041812 A1
(43) Pub. Date: Feb. 12, 2026

(54) ADHESIVE COMPOSITIONS WITH TUNABLE POROSITY AND ACIDITY CONTENT AND METHODS OF USE THEREOF

(71) Applicant: RevBio, Inc., Lowell, MA (US)

(72) Inventors: George W. Kay, Sharon, MA (US); Brian J. Hess, Charlestown, MA (US); Rahul P. Jadia, Lowell, MA (US); Brittany McDonough, Nashua, NH (US); Molly E. Shutt, Saugus, MA (US)

(21) Appl. No.: 19/099,185

(22) PCT Filed: Jul. 28, 2023
(86) PCT No.: PCT/US2023/029031

§ 371 Date: Jan. 28, 2025

Related U.S. Application Data
(60) Provisional application No. 63/369,690, filed on Jul. 28, 2022.

(51) Int. Cl. A61L 24/00; A61K 6/60; A61K 6/70; A61L 24/04; A61L 24/06; A61L 24/08; A61L 24/10

(52) U.S. Cl. CPC A61L 24/0036; A61K 6/60; A61K 6/70; A61L 24/046; A61L 24/06; A61L 24/08; A61L 24/102; A61L 24/104; A61L 24/108; A61L 2400/12; A61L 2430/02; A61L 2430/12

(57) ABSTRACT

Adhesive compositions including a multivalent metal salt, an multidentate acidic organic compound, and an aqueous medium are disclosed. The multivalent metal salt and multidentate acidic organic compound can include, as a mixture, a carbonate salt and/or carbonic acid. The aqueous medium can include pH adjusting agent such as sodium hydroxide. The adhesive composition, upon curing, can have a porosity of 10-50%. The adhesive composition, upon curing, can include a plurality of pores. The adhesive composition can be porous, and upon curing can have a plurality of pores having a pore size of between 20 μm to 200 μm. Methods of producing the adhesive composition are disclosed. Methods of using the adhesive composition are further disclosed.

### CROSS-REFERENCE TO RELATED APPLICATIONS

This application claims priority to U.S. Patent Application No. 63/369,690, filed Jul. 28, 2022, the entire contents of which are incorporated herein by reference in their entirety.

### BACKGROUND

The bones of the skeleton are metabolically active organs that undergo continuous modeling, remodeling, and repair throughout life as they perform their mechanical and physiological functions. Bone regenerative procedures and materials used for treatments for fractures, voids or other deficiencies of conditions rely on these internal processes and their control mechanisms to reach successful resolution of the problems presented for treatment. The processes involved in bone metabolism and repair are complex and involve physical-chemical influences such as the systemic acid-base balance, endocrine control at the systemic level through hormones (e.g., calcitonin, parathyroid hormone, growth hormone, corticosteroids, and others), local influences by locally acting hormones such as the prostaglandins of the E series, and paracrine signaling at cell size distances. The processes involved in bone metabolism also depend on the chemical composition of the mineral component of bone itself, bone apatite. Bone apatite approximates the composition and structure of stoichiometric hydroxyapatite compound, Ca₁₀(PO₄)₆(OH)₂, the most thermodynamically stable calcium phosphate phase in vertebrate body; however, some of the eighteen ions making up the crystal lattice, i.e., 10 Ca²⁺, 6(PO₄)³⁻, and 2 OH—, are occasionally substituted in the lattice by common ions present in the body, those most frequently being the cations Mg²⁺, Na⁺, K⁺, substituting for calcium, and the anions (CO₃)²⁻ and Cl⁻ substituting for either phosphate or hydroxide. These substitutions influence the chemical and physiological properties of the bone mineral and consequently bone tissue itself.

### SUMMARY

The present disclosure features, inter alia, compositions and methods for the regeneration of bone tissue, induction of osteoblast formation, and treatment of one or more diseases or disorders. In an aspect, the present disclosure features an adhesive composition including a multivalent metal salt, a multidentate acidic organic compound, a carbonate salt, and an aqueous medium. The adhesive composition, upon curing, can have a porosity of 10-50%.

In another aspect, the present disclosures features an adhesive composition including a multivalent metal salt, an multidentate acidic organic compound, a carbonate salt, and an aqueous medium. The adhesive composition, upon curing, can have a plurality of pores.

In another aspect, the present disclosures features an adhesive composition including a multivalent metal salt, an acidic compound, a carbonate salt, and an aqueous medium which upon mixing generates carbonic acid. The in situ carbonic acid dissociates into to carbon dioxide and water, creating a plurality of carbon dioxide gas bubbles within the reaction mixture. The gas bubbles are retained within the adhesive composition upon curing as plurality of pores.

In another aspect, the present disclosure features a porous adhesive composition including a multivalent metal salt, an multidentate acidic organic compound, a carbonate salt, and an aqueous medium. The porous adhesive composition, upon curing, can have a plurality of pores having a pore size of between 20 μm to 200 μm.

In another aspect, the present disclosure features a porous adhesive composition including a powdered multivalent metal salt, an multidentate acidic organic compound, an aqueous medium, and a granular material. In some embodiments the powdered multivalent metal salt when put in contact with and mixed with the aqueous material produces an adhesive viscous fluid which can serve as a binder partially filling the intergranular spaces of the mixture. In some embodiments the binder volume does not exceed 36% of the volume of the packed dry solid granular material alone. In some embodiments the total volume of the binder fluid, when compared to the total volume of the granules is in the range of 15 percent and 36 percent of the volume of the packed dry solid granular material alone. In some embodiments, the binder volume comprises between about 15 and about 19 percent, or between about 17 and about 21 percent, or between about 19 and about 23 percent, or between about 21 and 25 percent, between about 23 and about 27 percent, or between about 25 and about 29 percent, or between about 27 and about 31 percent, or between about 29 and 33 percent, between about 31 and about 35 percent, or between about 33 and about 36 percent of the volume of the packed dry solid granular material alone. In some embodiments the volume of the binder provided is sufficient to generate a mixture which is cohesive and yet is insufficient to completely fill the intergranular voids of the composition mixture. In some embodiments the voids in the intergranular spaces constitute porosity within the substance of the composition mixture. In some embodiments the voids in the intergranular spaces are continuous, branched, or interconnected.

In another aspect, the present disclosure features a porous adhesive composition including a powdered multivalent metal salt, an multidentate acidic organic compound, an aqueous medium, and a granular material. In some embodiments the powdered multivalent metal salt when put in contact with and mixed with the aqueous material produces an adhesive viscous fluid which can serve as a binder partially filling the intergranular spaces of the mixture. In some embodiments the binder volume does not exceed approximately 36% of the volume of the total combined adhesive mixture including the liquid binder and the packed dry solid granular material alone. In some embodiments the volume of the binder provided is sufficient to generate a mixture which is cohesive but is insufficient to fill the intergranular voids of the mixture. In some embodiments the voids in the intergranular spaces constitute porosity within the substance of the mixture. In some embodiments the voids in the intergranular spaces are continuous, branched, or interconnected.

In another aspect, the present disclosure features a porous adhesive composition including a powdered multivalent metal salt, an multidentate acidic organic compound, an aqueous medium, and a granular material. In some embodiments, the particles of the granular material are themselves porous.

In another aspect, the present disclosure features a porous adhesive composition including a powdered multivalent metal salt, an multidentate acidic organic compound, an aqueous medium, and a granular material. In some embodiments the granular material is composed of particles of similar composition. In some embodiments, the granular material comprises granules of different compositions. In some embodiments, a portion of the granules include materials that are more soluble than those comprising other granules.

In another aspect, the present disclosure features a porous adhesive composition including a powdered multivalent metal salt, an multidentate acidic organic compound, an aqueous medium, and a granular material. In some embodiments the granular material is composed of particles of similar composition. In some embodiments the granular material comprises granules of different compositions. In some embodiments some of the granules are composed of materials that are significantly more soluble than those comprising the binder. In another aspect, the present disclosure features a porous adhesive composition including a powdered multivalent metal salt, an multidentate acidic organic compound, an aqueous medium, and a granular material. In some embodiments the granular material is composed of particles of similar composition. In some embodiments the granular material comprises granules of different compositions. In some embodiments some of the granules are composed of materials that are significantly less soluble than those comprising the binder.

In another aspect, the present disclosure features a porous adhesive composition including a powdered multivalent metal salt, an multidentate acidic organic compound, an aqueous medium, and a granular material. In some embodiments some of the granules comprise an additive or a plurality of additives. In some embodiments those additives diffuse out of the granules. In some embodiments the additives do not diffuse out of the granules. In some of the embodiments the additives are released upon dissolution of the granules. In some of the embodiments the additives are released upon resorption of the granules.

In another aspect, the present disclosure features a porous adhesive composition including a powdered multivalent metal salt, an multidentate acidic organic compound, an aqueous medium, and a granular material. In some embodiments the adhesive liquid binder comprises an additive or a plurality of additives. In some embodiments those additives diffuse out of the binder once it has solidified. In some embodiments the additives do not diffuse out of the solidified binder. In some of the embodiments the additives are released upon dissolution of the gradual dissolution of the binder. In some of the embodiments the additives are released upon gradual resorption of the binder.

In another aspect, the present disclosure features a porous adhesive composition including a multivalent metal salt, an multidentate acidic organic compound, an aqueous medium and a pH adjusting agent. In some embodiments the pH adjusting agent is a base used to raise the pH of the reaction mixture leading the cured state of the composition. In some embodiments the pH adjusting agent is a hydroxide of a Group 1 element. In some embodiments the pH adjusting agent is sodium hydroxide. In some embodiments the pH adjusting agent is a weak acid salt of Group 1 element. In some embodiments, the pH adjusting agent is sodium carbonate. In some embodiments, the pH adjusting agent is tribasic sodium citrate. In some embodiments, the pH adjusting agent is sodium bicarbonate.

In another aspect, the present disclosure features a porous adhesive composition including a multivalent metal salt, an multidentate acidic organic compound, an aqueous medium and an acidity reducing agent. In some embodiments the pH adjusting agent is a base used to raise the pH. In some embodiments the pH adjusting agent is an oxide of a Group 2 element. In some embodiments the pH adjusting agent is calcium oxide. In some embodiments the pH adjusting agent is a hydroxide of a Group 2 element. In some embodiments the pH adjusting agent is magnesium hydroxide. In some embodiments the pH adjusting agent is a weak acid salt of a Group 2 element. In some embodiments, the pH adjusting agent is calcium carbonate. In some embodiments, the pH adjusting agent is strontium carbonate. In some embodiments, the pH adjusting agent is tribasic calcium citrate.

In some embodiments, the adhesive composition may include an acidity adjusting agent. As disclosed herein, the pH of the adhesive composition might in some embodiments comprising the multidentate organic acidic compound of Formula I (phosphoserine) and the alkaline multivalent metal salt which comprises mixed alkaline calcium phosphates, i.e., tetracalcium phosphate (TTCP), α-tricalcium phosphate (α-TCP), and hydroxyapatite (HA), is acidic immediately following activation, i.e., mixing of the powdered precursors and the aqueous medium, as a result of differential dissolution rate of the acidic component being much higher than that of the alkaline component, reaching pH values of 5.3, or lower. This transient decrease in pH is followed by a return to neutral pH upon curing; however, the high acidity of this implantable material can damage host tissues, resulting in high cytotoxicity and low survival rates (20%) of neighboring cells. The acidity adjusting agent can be any suitable compound that can be used to adjust the pH a desired amount. In certain embodiments, the acidity adjusting agent is a basic acidity adjusting agent, e.g., an agent with a pH greater than 7. The basic acidity adjusting agent can be any aqueous soluble basic compound including, but not limited to, the oxide and hydroxide salts of the Group I or Group II elements, e.g., alkali metal hydroxide, an alkali metal oxide, an alkaline earth metal hydroxide, an alkaline earth metal oxide, or a combination thereof. For example, the basic acidity adjusting agent can be selected from the group consisting of sodium hydroxide, potassium hydroxide, lithium hydroxide, magnesium hydroxide, or a combination thereof. In particular embodiments, the basic acidity adjusting agent is sodium hydroxide. In some embodiments, the aqueous medium contains the acidity adjusting agent. In specific embodiments, the aqueous medium is sodium hydroxide. In some embodiments of any and all aspects of the present disclosure, the acidity adjusting agent is sodium hydroxide that has a concentration between 0.5 and 5 M. The early return to physiological pH realized by inclusion of the acidity adjusting agent may be associated with the observed improved biocompatibility.

In another aspect, the present disclosure features a porous adhesive composition including a multivalent metal salt, an multidentate acidic organic compound, an aqueous medium and an acidity reducing agent. In some embodiments, the multivalent metal salt is an alkaline salt, e.g., tetracalcium phosphate, tribasic calcium citrate, calcium carbonate, calcium metasilicate, etc. In some embodiments the rate of acidity adjustment of the composition by a powdered acidity adjusting agent is controlled by the particle size profile of the acidity adjusting agent or combination of agents. Without wishing to be bound by any particular theory, the reaction between a solid and a surrounding liquid is at least in part influenced by the contacting surface area between the two, e.g., proportional: consequently, since the same mass of smaller particles must have a larger surface area than that composed of larger particles, the reaction rate, under otherwise identical conditions, will be higher when the particles are smaller. In some embodiments, when more rapid neutralization of acidity of the reaction mixture is desired, the alkaline multivalent metal salt constituent of the composition is designed to comprise a greater proportion of smaller particles, i.e., powders or granules, than otherwise specified. In some embodiments a specific alkaline multivalent metal salt component, e.g., calcium carbonate, calcium silicate or calcium citrate, may be specifically selected to participate in the reaction as a smaller-particle component of the composition. In some embodiments the selection of a specific multivalent metal salt to be compounded as a larger particle size profile compared to other multivalent metal salts or salts within the formulation may accelerate the appearance of certain properties, e.g., rise in porosity, increase in lubricity, increase in pH, etc., as a consequence of that design choice. In some embodiments the selection of a specific multivalent metal salt to be compounded as a larger particle size profile compared to other multivalent metal salts or salts within the formulation may decelerate the appearance of certain properties, e.g., rise in porosity, increase in lubricity, increase in pH, etc., as a consequence of that design choice.

In another aspect, the present disclosure features a porous adhesive composition including a multivalent metal salt, an multidentate acidic organic compound, an aqueous medium and an acidity reducing agent. In some embodiments, the multivalent metal salt is an alkaline salt. In some embodiments, the rate of acidity adjustment of the composition by a powdered pH adjusting agent is controlled by the particle size profile of the pH adjusting agent.

In another aspect, the present disclosure features a porous adhesive composition including a multivalent metal salt, an multidentate acidic organic compound, an aqueous medium and an acidity reducing agent. In some embodiments the pH adjusting agent is a base used to raise the pH. In some embodiments, the pH adjusting agent is a highly water-soluble compound.

In some embodiments, the pH adjusting agent, prior to the activation of the reaction generating the adhesive composition, is dissolved in the aqueous medium. In some embodiments, the pH adjusting agent dissolved in the aqueous medium is sodium hydroxide. In some embodiments, the pH adjusting agent dissolved in the aqueous medium is a polybasic sodium salt of the amino acid phosphoserine.

In another aspect, the present disclosure features a porous adhesive composition including a multivalent metal salt, an multidentate acidic organic compound, an aqueous medium and an acidity reducing agent. In some embodiments the pH adjusting agent is a base used to raise the pH. In some embodiments the pH adjusting agent is a highly water-soluble compound.

In some embodiments the pH adjusting agent prior to the activation of the reaction generating the adhesive composition is included among the powdered components. In some embodiments the pH adjusting agent included among the powdered components is tribasic sodium citrate. In some embodiments the pH adjusting agent included among the powdered components is sodium carbonate. In some embodiments the pH adjusting agent included among the powdered components is a polybasic sodium salt of the amino acid phosphoserine.

In another aspect, the present disclosure features a porous adhesive composition comprising a multivalent metal salt, an multidentate acidic organic compound, an aqueous medium and a porogen. In some embodiments, the porogen comprises sodium, calcium, potassium, magnesium, lithium, or carbon. In some embodiments, the porogen is a carbonate salt. In some embodiments, the porogen is a bicarbonate salt, i.e., a hydrogen carbonate salt. As used herein, “carbonate” refers to either carbonate or bicarbonate, i.e., hydrogen carbonate.

In another aspect, the present disclosure features an adhesive composition including a multivalent metal salt, an multidentate acidic organic compound, a carbonate salt, and an aqueous medium.

In another aspect, the present disclosure features an adhesive composition including a multivalent metal salt, an multidentate acidic organic compound, a carbonate salt, and an aqueous medium including a pH adjusting agent. In some embodiments the carbonate salt is nanocrystalline calcium carbonate.

In another aspect, the present disclosure features an adhesive composition including a multivalent metal salt, an multidentate acidic organic compound, and an additive that accelerates dissolution of the cured composition. In some embodiments the dissolution accelerating additive is a highly soluble salt of a Group 1 element. In some embodiments the additive is a sodium salt.

In another aspect, the present disclosure features a method of preparing an adhesive composition. The method may include providing a mixture of a multivalent metal salt, multidentate acidic organic compound, and a carbonate salt, contacting the mixture with an aqueous medium, and adding a pH adjusting agent to the mixture to bring a pH of the mixture to a value between 5 to 9, thereby preparing the adhesive composition. In some embodiments, the pH adjusting agent brings the pH of the mixture to a value between 5.7 to 8. In some embodiments, a plurality of pH adjusting agents brings the pH of the mixture to a value between 5.7 to 8.

In another aspect, the present disclosure features a method of preparing an adhesive composition. The method may include providing a mixture of a multivalent metal salt, multidentate acidic organic compound, and a carbonate salt, contacting the mixture with an aqueous medium; and adding a pH adjusting agent to the mixture to evolve a gas from the mixture. In some embodiments, the evolved gas is derived from dissociating carbonic acid. In some embodiments, the pH adjusting agent also acts as a porogen. The adhesive composition, upon curing, can have a porosity of 10-50%.

In another aspect, the present disclosure features a method of preparing an adhesive composition. The method may include providing a mixture of a multivalent metal salt, multidentate acidic organic compound, and a carbonate salt, contacting the mixture with an aqueous medium, and adding a pH adjusting agent to the mixture to evolve a gas from the mixture. The adhesive composition, upon curing, can have a plurality of pores having a pore size of between 20 m to 200 m, thereby preparing the adhesive composition.

In some embodiments, the pH adjusting agent is a basic pH adjusting agent. In some embodiments, the basic pH adjusting agent may include an alkali metal hydroxide, an alkali metal oxide, an alkaline earth metal hydroxide, an alkaline earth metal oxide, or a combination thereof. For example, the basic pH adjusting agent may be selected from the group consisting of sodium hydroxide, potassium hydroxide, lithium hydroxide, magnesium hydroxide, or a combination thereof. In some embodiments, the pH adjusting agent may be a weak acid salt, e.g., a carbonate or citrate salt. In some embodiments, the basic pH adjusting agent is sodium hydroxide.

In another aspect, the present disclosure features a method of generating or regenerating bone tissue. The method may include preparing an adhesive composition comprising a multivalent metal salt, an multidentate acidic organic compound, and a carbonate salt in an aqueous medium, applying the adhesive composition to a site (e.g., into or onto bone, or in between bones or bone fragments), and allowing the adhesive composition to harden, cure, or be resorbed by bone, the adhesive composition, upon curing, having a porosity of 10-50%, thereby generating or regenerating bone tissue.

In another aspect, the present disclosure features a method of inducing osteoblast formation. The method may include preparing an adhesive composition comprising a multivalent metal salt, an multidentate acidic organic compound, and a carbonate salt in an aqueous medium, applying the adhesive composition to a site (e.g., into or onto bone, or in between bones or bone fragments), and allowing the adhesive composition to harden, cure, or be resorbed by bone, the adhesive composition, upon curing, having a porosity of 10-50%, thereby inducing osteoblast formation.

In another aspect, the present disclosure features a method of treating disabilities consequent to bone disease or disorder in a subject, e.g., following resection of affected bone.

The method may include preparing an adhesive composition comprising a multivalent metal salt, an multidentate acidic organic compound, and a carbonate salt in an aqueous medium, applying the adhesive composition to a site (e.g., into or onto bone, or in between bones or bone fragments), and allowing the adhesive composition to harden, cure, or be resorbed by bone, the adhesive composition, upon curing, having a porosity of 10-50%, thereby treating or preventing a bone disease or disorder in a subject.

In another aspect, the present disclosure features a method of inducing expression of a cell proliferation marker, e.g., MKI67. The method may include preparing an adhesive composition comprising a multivalent metal salt, an multidentate acidic organic compound, and a carbonate salt in an aqueous medium, applying the adhesive composition to a site (e.g., into or onto bone, or in between bones), and allowing the adhesive composition to harden, cure, or be resorbed by bone, the adhesive composition, upon curing, having a porosity of 10-50%, thereby inducing expression of a cell proliferation marker, e.g., MKI67.

In another aspect, the present disclosure features a method of inducing expression of Bone Morphogenetic protein-2 (BMP2). The method may include preparing an adhesive composition comprising a multivalent metal salt, an multidentate acidic organic compound, and a carbonate salt in an aqueous medium, applying the adhesive composition to a site (e.g., into or onto bone, or in between bones or bone fragments), and allowing the adhesive composition to harden, cure, or be resorbed by bone, the adhesive composition, upon curing, having a porosity of 10-50%, thereby inducing expression of BMP2.

In another aspect, the present disclosure features a method of inducing expression of one or both of caspase-3 and caspase-9. The method may include preparing an adhesive composition comprising a multivalent metal salt, an organic phosphate compound, and a carbonate salt in an aqueous medium, applying the adhesive composition to a site (e.g., into or onto bone, or in between bones), and allowing the adhesive composition to harden, cure, or be resorbed by bone, the adhesive composition, upon curing, having a porosity of 10-50%, thereby inducing expression of one or both of caspase-3 and caspase-9.

In another aspect, the present disclosure features a method of inducing expression of Interleukin-6 (IL-6). The method may include preparing an adhesive composition comprising a multivalent metal salt, an organic phosphate compound, and a carbonate salt in an aqueous medium, applying the adhesive composition to a site (e.g., into or onto bone, or in between bones), and allowing the adhesive composition to harden, cure, or be resorbed by bone, the adhesive composition, upon curing, having a porosity of 10-50%, thereby inducing expression of IL-6.

In another aspect, the present disclosure features a method of inducing expression of tumor necrosis factor alpha (TNF-α). The method may include preparing an adhesive composition comprising a multivalent metal salt, an organic phosphate compound, and a carbonate salt in an aqueous medium, applying the adhesive composition to a site (e.g., into or onto bone, or in between bones), and allowing the adhesive composition to harden, cure, or be resorbed by hone, the adhesive composition, upon curing, having a porosity of 10-50%, thereby inducing expression of TNF-α.

In some embodiments, the adhesive composition, upon curing, can have a density between 0.75 g/cm³ to 1.40 g/cm³, e.g., between 0.75 g/cm³ to 1.40 g/cm³, between 0.80 g/cm³ to 1.35 g/cm³, between 0.85 g/cm³ to 1.30 g/cm³, between 0.90 g/cm³ to 1.25 g/cm³, between 0.95 g/cm³ to 1.20 g/cm³, between 1.00 g/cm³ to 1.15 g/cm³, or between 1.05 g/cm³ to 1.10 g/cm³, e.g., between 0.75 g/cm³ to 1.00 g/cm³, between 0.80 g/cm³ to 1.05 g/cm³, between 0.85 g/cm³ to 1.10 g/cm³, between 0.90 g/cm³ to 1.15 g/cm³, between 0.95 g/cm³ to 1.20 g/cm³, between 1.00 g/cm³ to 1.25 g/cm³, between 1.05 g/cm³ to 1.30 g/cm³, between 1.10 g/cm³ to 1.35 g/cm³, or between 1.15 g/cm³ to 1.40 g/cm³, e.g., about 0.75 g/cm³, about 0.80 g/cm³, about 0.85 g/cm³, about 0.90 g/cm³, about 0.95 g/cm³, about 1.00 g/cm³, about 1.05 g/cm³, about 1.10 g/cm³, about 1.15 g/cm³, about 1.20 g/cm³, about 1.25 g/cm³, about 1.30 g/cm³, about 1.35 g/cm³, or about 1.40 g/cm³.

In some embodiments, a pH of the adhesive composition in a tacky state is between 7 to 10. In some embodiments, a pH of the adhesive composition in a tacky state is between 5.7 to 6.5.

In some embodiments, the multivalent metal salt comprises calcium phosphate, e.g., tetracalcium phosphate or tricalcium phosphate, e.g., α-tricalcium phosphate or β-tricalcium phosphate, calcium citrate, calcium carbonate, magnesium phosphate, sodium silicate, calcium silicate, lithium phosphate, titanium phosphate, strontium phosphate, zinc phosphate, calcium oxide, magnesium oxide, calcium silicate, or a combination thereof.

In some embodiments of any and all aspects of the present disclosure, the multidentate acidic organic compound is a compound of Formula (I):

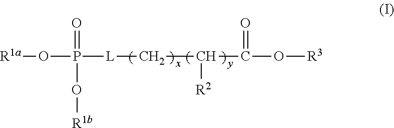

or a pharmaceutically acceptable salt thereof, wherein L is O, S, NH, or CH₂; each of R^{1a} and R^{1b} is independently H, optionally substituted alkyl, or optionally substituted aryl; R² is H, NR^{4a}R^{4b}, C(O)R⁵, or C(O)OR⁵; R³ is H, optionally substituted alkyl, or optionally substituted aryl; each of R^{4a} and R^{4a} is independently H, C(O)R⁶, or optionally substituted alkyl; R⁵ is H, optionally substituted alkyl, or optionally substituted aryl; R⁶ is optionally substituted alkyl or optionally substituted aryl; and each of x and y is independently 0, 1, 2, or 3. Phosphoscrine is exemplary of compounds of Formula (I).

In some embodiments, the multidentate acidic organic compound (e.g., a compound of Formula (I)) is present in an amount greater than or equal to about 10% (w/w) of the total composition. In some embodiments, the multidentate acidic organic compound (e.g., a compound of Formula (I)) is present in an amount greater than or equal to about 1% (w/w), about 2% (w/w), about 5% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 22.5% (w/w), about 25% (w/w), about 30% (w/w), about 35% (w/w), about 40% (w/w), about 45% (w/w), about 50% (w/w), or more of the total composition.

In some embodiments, the multidentate acidic organic compound (e.g., a compound of Formula (I)) is present in an amount greater than or equal to about 0.1% (w/w) of the composition. In some embodiments, the multidentate acidic organic compound (e.g., a compound of Formula (I)) is present in an amount greater than or equal to about 0.1% (w/w), about 0.5% (w/w), about 1% (w/w), about 3% (w/w), about 5% (w/w), about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w), or about 80% (w/w) of the composition.

In some embodiments, the multidentate acidic organic compound is present within the adhesive composition in an amount between 10% and 90% (w/w) of the total weight. In some embodiments, the amount of the multivalent metal salt (e.g., a calcium phosphate, e.g., tetracalcium phosphate, tricalcium phosphate, hydroxyapatite, or calcium oxide, calcium silicate, a magnesium phosphate, magnesium hydroxide, magnesium silicate, or a combination thereof) is in the range of about 10% to about 90%, about 15% to about 85%, about 20% to about 80%, about 30% to about 75%, about 40% to about 70%, or about 50% to about 65% w/w of the total composition. In other embodiments, the amount of the metal salt (e.g., a calcium phosphate or calcium oxide or a combination thereof) is in the range of about 5% to about 95%, about 10% to about 85%, about 15% to about 75%, about 20% to about 65%, about 25% to about 55%, or about 35% to about 50% w/w of the total composition.

In some embodiments, the aqueous medium comprises water, saliva, saline, serum, plasma, or blood.

In some embodiments of any and all aspects of the present disclosure the adhesive composition further comprises an additive. The additive can include a salt, filler, formulation base, viscosity modifier, abrasive, coloring agent, flavoring agent, or polymer. In some embodiments, the polymer may be poly(L-lactide), poly(D,L-lactide), polyglycolide, poly(ε-caprolactone), poly(teramethylglycolic-acid), poly(dioxanone), poly(hydroxybutyrate), poly(hydroxyvalerate), poly(lactide-co-glycolide), poly(glycolide-co-trimethylene carbonate), poly(glycolide-co-caprolactone), poly(glycolide-co-dioxanone-co-trimethylene-carbonate), poly(tetramethylglycolic-acid-co-dioxanone-co-trimethylenecarbonate), poly(glycolide-co-caprolactone-co-lactide-co-trimethylene-carbonate), poly(hydroxybutyrate-co-hydroxyvalerate), poly(methylmethacrylate), poly(acrylate), polyamines, polyamides, polyimidazoles, poly(vinyl-pyrrolidone), collagen, silk, chitosan, hyaluronic acid, gelatin, or a mixture thereof. In some embodiments, the additive is a pH adjusting agent. In some embodiments, the additive is a porogen.

In some embodiments of any and all aspects of the present disclosure, the carbonate salt comprises a carbonate salt of Group I or Group II elements, e.g., beryllium carbonate, beryllium bicarbonate, magnesium carbonate, magnesium bicarbonate, calcium carbonate, calcium bicarbonate, strontium carbonate, strontium bicarbonate, sodium carbonate, sodium bicarbonate, potassium carbonate, potassium bicarbonate, etc.

In some embodiments, the adhesive composition during the tacky state has a tack stress of between about 10 kPa and about 250 kPa after mixing with the aqueous medium. In some embodiments, the adhesive composition has a putty state for up to 15 minutes after mixing with the aqueous medium. In some embodiments, the adhesive composition during the putty state has a tack stress of between about 10 kPa and about 250 kPa after mixing with the aqueous medium.

In some embodiments, the adhesive composition has an adhesive strength upon curing of greater than 100 kPa. In some embodiments, the adhesive composition may exhibit an adhesive strength in the cement-like state in the range of about 100 kPa to about 12,000 kPa, depending on the application and the particular components and ratios of components in said adhesive compositions. In some embodiments, the adhesive strength of the adhesive composition in the cement-like state is between about 100 kPa and about 10,000 kPa, e.g., about 9,000 kPa, about 8,000 kPa, about 7,000 kPa, about 6,000 kPa, about 5,000 kPa, about 4,000 kPa, about 3,000 kPa, about 2,000 kPa, about 1,000 kPa, about 750 kPa, about 500 kPa, about 250 kPa, or about 200 kPa. In some embodiments, the adhesive strength of the adhesive composition in the cement-like state is between about 100 kPa, about 200 kPa, about 300 kPa, about 400 kPa, about 500 kPa, about 600 kPa, about 700 kPa, about 800 kPa, about 900 kPa, about 1,000 kPa, about 2,500 kPa, about 5,000 kPa, about 7,500 kPa, about 10,000 kPa or about 12,000 kPa. In some embodiments, the adhesive strength of the adhesive composition in the cement-like state is in the range of about 200 kPa and about 2,500 kPa. In some embodiments, the adhesive strength of the adhesive composition in the cement-like state is greater than 100 kPa.

In some embodiments of any and all aspects of the present disclosure, the multivalent metal compound is provided as a powder. For example, the mean particle size of the powder is about 0.0001 to about 1.000 mm, about 0.0005 to about 0.001 mm, about 0.001 to about 0.025 mm, about 0.005 to about 0.015 mm, about 0.001 to about 0.250 mm, about 0.005 to about 0.150 mm, about 0.250 to about 0.750 mm, about 0.25 to about 0.50 mm, about 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm. In some embodiments the multivalent metal powders may exhibit a minimum percentage of mass content of particles with particle size less than or equal to 0.090 mm, more specifically particles less than or equal to 0.045 mm, wherein the minimum percentage is at least 10 percentage. The particle size distribution may be multi-modal to include any combination of mean particle sizes as previously described. These granules may exhibit a mean granule size of about 0.050 mm to about 5 mm, about 0.100 to about 1.500 mm, about 0.125 to about 1.000 mm, about 0.125 to about 0.500 mm, about 0.125 to about 0.250 mm, about 0.250 to about 0.750 mm, about 0.250 to about 0.500 mm, about 0.500 to about 1.00 mm, about 0.500 to about 0.750 mm. The granule size distribution may be multi-modal to include any combination of mean granule sizes as previously described. In some embodiments, the granules may be supplied with a various proportion of porosity and a various size of internal pores. The pores may communicate with each other. The pores may communicate with granule surface. In some embodiments, the pores do not communicate with each other. In some embodiments, the pores do not communicate with granule surface. In some embodiments, varying sizes of said powders or granules may be used in the adhesive composition.

In some embodiments of any and all aspects of the present disclosure, the porogen (e.g., the carbonate salt) is provided in a solid particle form, such as a microparticle or nanoparticle. In an embodiment, the porogen (e.g., the carbonate salt) is provided in a nanoparticle form (e.g., having a diameter of about 10 nm to about 1000 nm). In an embodiment, the porogen (e.g., the carbonate salt) is provided in a microparticle form (e.g., having a diameter of about 10 m to about 1000 μm). In some embodiments, the porogen (e.g., the carbonate salt) includes a material supplied as nanoparticles, e.g., particles having an average diameter of 20 nm to 200 nm. In some embodiments, the resulting adhesive composition is porous and includes a plurality of pores, wherein said pores range in size from about 0.01 mm to about 1.0 mm.

In some embodiments of any and all aspects of the present disclosure, the pH adjusting agent is sodium hydroxide, having a molar concentration between 0.5 and 5 M. In some embodiments, inclusion of the pH adjusting agent brings the pH of the adhesive composition to between 5.7 to 8.

### BRIEF DESCRIPTION OF THE DRAWINGS

The accompanying drawings are not drawn to scale. In the drawings, each identical or nearly identical component that is illustrated in the various figures is represented by a like numeral. For purposes of clarity, not every component may be labeled in every drawing. In the drawings:

FIGS. 1A-1B illustrate the pH of the elution medium of compositions according to this disclosure with varied particle sizes of calcium carbonate particles. FIG. 1A illustrates the pH of the elution medium as a function of composition, with each bar being a different time post-deposition. FIG. 1B illustrates the pH of the elution medium as a function of time, with each bar being a different composition.

FIGS. 2A-2B illustrate the pH of the elution medium of compositions according to this disclosure with varied particle sizes of multivalent metal salt particles. FIG. 2A illustrates the pH of the elution medium as a function of composition, with each bar being a different time post-deposition. FIG. 2B illustrates the pH of the elution medium as a function of time, with each bar being a different composition.

FIGS. 3A-3B illustrate the pH of the medium surrounding cured compositions according to this disclosure made with fines. FIG. 3A illustrates the pH of the medium surrounding cured compositions as a function of composition, with each bar being a different time post-deposition.

FIG. 3B illustrates the pH of the medium surrounding cured compositions as a function of time, with each bar being a different composition.

FIGS. 4A-4D illustrate the gene expression of the MK167 marker as a metric for bone marrow stromal cell proliferation using adhesive compositions of this disclosure relative to control compositions. FIG. 4A illustrates the gene expression of the MK167 marker one day following application of the various compositions. FIG. 4B illustrates the gene expression of the MKI67 marker three days following application of the various compositions. FIG. 4C illustrates the gene expression of the MKI67 marker five days following application of the various compositions. FIG. 4D illustrates the gene expression of the MKI67 marker seven days following application of the various compositions.

FIGS. 5A-5D illustrate the gene expression of Bone Morphogenetic protein-2 (BMP2) as a metric for bone marrow stromal cell differentiation using adhesive compositions of this disclosure relative to control compositions. FIG. 5A illustrates the gene expression of BMP2 one day following application of the various compositions. FIG. 5B illustrates the gene expression of BMP2 three days following application of the various compositions. FIG. 5C illustrates the gene expression of BMP2 five days following application of the various compositions. FIG. 5D illustrates the gene expression of BMP2 seven days following application of the various compositions.

FIGS. 6A-6D illustrate an evaluation of collagen deposition via expression of the COL1A1 gene following application of compositions according to this disclosure. FIG. 6A illustrates the gene expression of COL1A1 one day following application of the various compositions. FIG. 6B illustrates the gene expression of COL1A1 three days following application of the various compositions. FIG. 6C illustrates the gene expression of COL1A1 five days following application of the various compositions. FIG. 6D illustrates the gene expression of COLA1 seven days following application of the various compositions.

FIGS. 7A-7D illustrate an evaluation of cell apoptosis via expression of the Caspase-3 gene following application of compositions according to this disclosure. FIG. 7A illustrates the gene expression of Caspase-3 one day following application of the various compositions. FIG. 7B illustrates the gene expression of Caspase-3 three days following application of the various compositions. FIG. 7C illustrates the gene expression of Caspase-3 five days following application of the various compositions. FIG. 7D illustrates the gene expression of Caspase-3 seven days following application of the various compositions.

FIGS. 8A-8D illustrate an evaluation of cell apoptosis via expression of the Caspase-9 gene following application of compositions according to this disclosure. FIG. 8A illustrates the gene expression of Caspase-9 one day following application of the various compositions. FIG. 8B illustrates the gene expression of Caspase-9 three days following application of the various compositions. FIG. 8C illustrates the gene expression of Caspase-9 five days following application of the various compositions. FIG. 8D illustrates the gene expression of Caspase-9 seven days following application of the various compositions.

FIGS. 9A-9D illustrate an evaluation of biomineralization via expression of the ALPL gene following application of compositions according to this disclosure. FIG. 9A illustrates the gene expression of ALPL one day following application of the various compositions. FIG. 9B illustrates the gene expression of ALPL three days following application of the various compositions. FIG. 9C illustrates the gene expression of ALPL five days following application of the various compositions. FIG. 9D illustrates the gene expression of ALPL seven days following application of the various compositions.

FIGS. 10A-10D illustrate the gene expression of Vascular Endothelial Growth factor (VEGF) as a metric for bone marrow stromal cell angiogenesis using adhesive compositions of this disclosure relative to control compositions. FIG. 10A illustrates the gene expression of VEGF one day following application of the various compositions. FIG. 10B illustrates the gene expression of VEGF three days following application of the various compositions. FIG. 10C illustrates the gene expression of VEGF five days following application of the various compositions. FIG. 10D illustrates the gene expression of VEGF seven days following application of the various compositions.

FIG. 11A-11B illustrate Cone beam computed tomography (CBCT) images of compositions of this disclosure at 3 and 8 weeks post deposition. The white arrow indicates the lateral condyle indicating implantation site. FIG. 11A illustrates CBCT images of compositions of this disclosure at 3 weeks post deposition. FIG. 11B illustrates CBCT images of compositions of this disclosure at 8 weeks post deposition.

FIGS. 12A-12B illustrate histological stains of compositions of this disclosure at 3 and 8 weeks post deposition. FIG. 12A illustrates histological stains of compositions of this disclosure at 3 weeks post deposition. FIG. 12B illustrates histological stains of compositions of this disclosure at 8 weeks post deposition.

FIG. 13 illustrates clinical and CBCT images of the left side of the jaw in a first canine patient with an extracted tooth replaced by an implant secured with compositions of this disclosure. The images shown are pre-operative, during the operation, and immediately following the operation.

FIG. 14 illustrates clinical and CBCT images of the canine jaw in FIG. 13 at various time points post operation.

FIG. 15 illustrates histological images of the canine jaw in FIGS. 13 and 14 at the site of implantation.

FIG. 16 illustrates clinical and CBCT images of the right side of the jaw in the first canine patient with an extracted tooth replaced by an implant secured with compositions of this disclosure. The images shown are pre-operative, during the operation, and immediately following the operation.

FIG. 17 illustrates clinical and CBCT images of the canine jaw in FIG. 16 at various time points post operation.

FIG. 18 illustrates histological images of the canine jaw in FIGS. 16 and 17 at the site of implantation.

FIG. 19 illustrates clinical and CBCT images of the left side of the jaw in a second canine patient with an extracted tooth replaced by an implant secured with compositions of this disclosure. The images shown are pre-operative, during the operation, and immediately following the operation.

FIG. 20 illustrates clinical and CBCT images of the canine jaw in FIG. 19 at various time points post operation.

FIG. 21 illustrates histological images of the canine jaw in FIGS. 19 and 20 at the site of implantation.

FIG. 22 illustrates clinical and CBCT images of the right side of the jaw in the second canine patient with an extracted tooth replaced by an implant secured with compositions of this disclosure. The images shown are pre-operative, during the operation, and immediately following the operation.

FIG. 23 illustrates clinical and CBCT images of the canine jaw in FIG. 22 at various time points post operation.

FIG. 24 illustrates histological images of the canine jaw in FIGS. 22 and 23 at the site of implantation.

FIG. 25 illustrates clinical and CBCT images of the left side of the jaw in a third canine patient with an extracted tooth replaced by an implant secured with compositions of this disclosure. The images shown are pre-operative, during the operation, and immediately following the operation.

FIG. 26 illustrates clinical and CBCT images of the canine jaw in FIG. 25 at various time points post operation.

FIG. 27 illustrates histological images of the canine jaw in FIGS. 25 and 26 at the site of implantation.

FIG. 28 illustrates clinical and CBCT images of the right side of the jaw in the third canine patient with an extracted tooth replaced by an implant secured with compositions of this disclosure. The images shown are pre-operative, during the operation, and immediately following the operation.

FIG. 29 illustrates clinical and CBCT images of the canine jaw in FIG. 28 at various time points post operation.

FIG. 30 illustrates histological images of the canine jaw in FIGS. 28 and 29 at the site of implantation.

FIG. 31 illustrates the pH of the elute over 48 hours when compositions of this disclosure were incorporated with one or more additives.

FIGS. 32A-32B illustrate a comparison of test cylinders made from adhesive compositions of this disclosure. FIG. 32A illustrates a micro-CT image of a test cylinder made using a pH-adjusted adhesive composition having a resulting porosity of 30%. FIG. 32B illustrates a micro-CT image of a test cylinder made using a non-pH adjusted adhesive composition having a resulting porosity of 4%.

FIG. 33 illustrates a SEM micrograph of a cured and granulated pH-adjusted adhesive composition of this disclosure listing measured pore diameters.

FIG. 34 illustrates the pH changes of an aqueous background solution as the organic phosphate component elutes from the adhesive compositions disclosed herein over a two week period.

FIG. 35 illustrates the percentage cumulative release profiles of the organic phosphate component from dense (solid bars) and porous (checkered bars) adhesive compositions disclosed herein over a two week period.

FIG. 36 illustrates the percentage release profiles of the organic phosphate component from the adhesive compositions disclosed herein over a two week period.

FIGS. 37A-37D illustrate the procedure used to create an epiphyseal defect into the distal femur of a New Zealand white rabbit for evaluation of adhesive compositions of this disclosure.

FIG. 37A illustrates the incision, with a ruler for reference, made to expose the portion of the distal femur. FIG. 37B illustrates drilling into the exposed distal femur to produce the epiphyseal defect. FIG. 37C illustrates the shape of the resulting epiphyseal defect. FIG. 37D illustrates the application of the adhesive composition of this disclosure to the epiphyseal defect.

FIGS. 38A-38D illustrate eosinophilic trabecular bone formation and osteoid deposition at two different time periods following application of two different compositions of this disclosure. FIG. 38A illustrates the lack of eosinophilic trabecular bone formation and osteoid deposition at 3 weeks following application of a non-pH adjusted adhesive composition of this disclosure. FIG. 38B illustrates abundant eosinophilic trabecular bone formation and osteoid deposition at 3 weeks following application of a pH adjusted adhesive composition of this disclosure. FIG. 38C illustrates the lack of eosinophilic trabecular bone formation and osteoid deposition at 8 weeks following application of a non-pH adjusted adhesive composition of this disclosure. FIG. 38D illustrates abundant eosinophilic trabecular bone formation and osteoid deposition at 8 weeks following application of a pH adjusted adhesive composition of this disclosure.

FIGS. 39A-39C illustrate the procedure used to create a distal root defect into mandible of a canine for evaluation of adhesive compositions of this disclosure. FIG. 39A illustrates the creation of a three-wall defect in the distal root of the first molar. FIG. 39B illustrates the deposition of an adhesive composition into the distal root defect. FIG. 39C illustrates the soft tissue healing progress at six weeks following deposition of the adhesive composition into the distal root defect.

FIGS. 40A-40D illustrate radiographic images of the distal root defect site shown in FIGS. 42A-42C at different time point following implantation of the adhesive composition into the distal root defect. FIG. 40A illustrates the adhesive composition implantation immediately post-procedure. FIG. 40B illustrates the adhesive composition implantation and its resorption at two weeks following implantation. FIG. 40C illustrates the bone substitution at four weeks following implantation. FIG. 40D illustrates the bone substitution at six weeks following implantation.

FIGS. 41A-41B illustrate the pH of the elution medium of compositions according to this disclosure with calcium citrate and particles and citric acid. FIG. 41A illustrates the pH of the elution medium as a function of composition, with each bar being a different time post-deposition. FIG. 41B illustrates the pH of the elution medium as a function of time, with each bar being a different composition.

FIGS. 42A-42B illustrate the pH of the elution medium of compositions according to this disclosure having higher porosity. FIG. 42A illustrates the pH of the elution medium as a function of composition, with each bar being a different time post-deposition. FIG. 42B illustrates the pH of the elution medium as a function of time, with each bar being a different composition.

FIG. 43 illustrates the pH of the elution medium of compositions according to this disclosure with α-tricalcium phosphate and calcium silicate particles.

### DETAILED DESCRIPTION

Cohesive, bone-adhesive, bone-conductive, and bone-regenerative compositions comprising a multivalent metal phosphate and a multidentate organic acid are described herein. The initial tack strength of the composition allows for immediate stabilization of bone fragments and implants contacting bone or metal surface. In vivo, the lasting bond formed during curing becomes stronger over the ensuing days, weeks and months and persists until the material is gradually degraded while being substantially replaced by newly formed bone osseointegrating the stabilized members.

The present disclosure features adhesive compositions and methods of use thereof, wherein the porosity and the pH, in isolation or simultaneously, of the mixture forming the composition is adjusted to provide improved biocompatibility and to modulate new bone growth. In an embodiment, the compositions described herein exhibit the advantageous property of robust adhesive behavior toward bone and other materials, (e.g., titanium and other metals), followed by bone regenerative behavior wherein the adhesive composition is gradually dissolved, resorbed and replaced with new bone over time while maintaining deposit volume. In some embodiments, the adhesive composition may be used to occlude access to the bone or bone wound surface by undesirable factors.

It has been observed that the pH of some adhesive compositions at the time of activation by combining the powdered and liquid precursors and mixing is unphysiologically low (pH-5) but returns to physiologically appropriate levels later during the curing process (pH-7). However, the passing stage of unphysiologically low pH, i.e., high acidity, may be cytotoxic to the tissues surrounding the composition at that phase of curing. Without wishing to be bound by any particular theory, the solubility of the acidic component of the adhesive composition, e.g., the multidentate acidic organic compound, phosphoserine, is approximately 45,000× greater than the solubility of the alkaline component on molarity basis at neutral pH, e.g., pH of 7, suggesting that inclusion of dissolved or readily soluble alkali compounds to increase the pH of the surrounding milieu may modulate the initial drop in pH to less unphysiological levels. One of the goals of the present disclosure is to enhance the biocompatibility of exemplary adhesive compositions. As the pH is a measure of the acidity, its value being the logarithm of the hydrogen ion concentration, phrases pH adjustment, pH modification, acidity adjustment, and acidity modification will be used interchangeably herein and will refer to the process of altering the hydrogen cation availability within the adhesive composition for diffusion into its immediate environment. The modifications described herein may result in the either lowering or increasing the availability of hydrogen cations for diffusion into the immediate environment of the adhesive composition; however, within the specific context of clinical applications considered and illustrated within this disclosure, it may be desirable to lower the availability of hydrogen cation for release. In an embodiment, modification of the acidity of a substance, i.e., its acid content, and modification of pH may not be linearly related.

A plot of pH versus an amount of a titrant, NaOH, illustrates the typically lower slope in the pH rise in proximity to one of the pKₐ of the acid being titrated. This behavior may also observed within the system of an adhesive composition, resulting in small increase in the pH while the pH adjustment methods are applied; however, the acidity of an adhesive composition may be altered quite substantially, as reflected by the amount of the base equivalents added by the methods disclosed herein. In an embodiment, exemplary adhesive compositions described herein exhibit similar pH values for their elution media during the early phases of the reaction, i.e., the first 15 minutes to one hour: between 5.4 and 5.9, reflecting the pKₐ value of the primary compound eluting from the composition at the corresponding early time points, phosphoserine, with published values of pKa in the range of 5.6 to 5.8. The purpose served by the pH adjustment, i.e., acidity adjustment, methods disclosed herein is to lower the amount of acid that must be buffered and neutralized by the surrounding host tissues during the early phases of the curing reaction of the adhesive composition in order to prevent tissue damage collateral to that buffering and neutralization following implantation, for example. The less kinetically favored, i.e., by virtue of low solubility, reactions between the multivalent metal salts, e.g., TTCP, α-TCP, etc., and the readily soluble multidentate organic acidic compounds, e.g., phosphoserine, citric acid, etc., continue to proceed and ultimately result in nearly neutral pH of the elution media, whether the formulation uses the methods disclosed herein or not.

### Chemical Definitions

Definitions of specific functional groups and chemical terms are described in more detail below. The chemical elements are identified in accordance with the Periodic Table of the Elements, CAS version, Handbook of Chemistry and Physics, 75′ Ed., inside cover, and specific functional groups are generally defined as described therein.

“Alkyl” refers to a radical of a straight-chain or branched saturated hydrocarbon group. In some embodiments, an alkyl group has 1 to 3 carbon atoms (“C₁₋₃ alkyl”). In some embodiments, an alkyl group has 1 carbon atom (“C₁ alkyl”). Examples of C_{1 6} alkyl groups include methyl (C₁) and propyl (C₃). Alkyl groups disclosed herein may be substituted or unsubstituted.

As used herein, “alkylene,” refers to a divalent radical of an alkyl group. When a range or number of carbons is provided for a particular “alkylene” group, it is understood that the range or number refers to the range or number of carbons in the linear carbon divalent chain.

The term “heteroalkyl,” as used herein, refers to an alkyl group, as defined herein, which further comprises 1 or more (e.g., 1 or 2) heteroatoms (e.g., non-ionizable heteroatoms, e.g., oxygen) within the parent chain, wherein the one or more heteroatoms is inserted between adjacent carbon atoms within the parent carbon chain and/or one or more heteroatoms is inserted between a carbon atom and the parent molecule, i.e., between the point of attachment.

As used herein, “alkylene,” and “heteroalkylene,” refer to a divalent radical of an alkyl and heteroalkyl group respectively. When a range or number of carbons is provided for a particular “alkylene” or “heteroalkylene” group, it is understood that the range or number refers to the range or number of carbons in the linear carbon divalent chain.

As used herein, “carboxyl” refers to —C(O)OH.

As used herein, “phosphonyl” refers to —P(O)(OH)₂.

As used herein, “aryl” refers to a functional group or substituent derived from an aromatic ring. In some embodiments, an aryl may be derived from an aromatic hydrocarbon.

Exemplary aryl groups include, but are not limited to, phenyl, naphthyl, thienyl, indolyl, and xylyl. Aryl groups disclosed herein may be substituted or unsubstituted.

As used herein, “hydroxy” refers to —OH.

As used herein “thiol” refers to a sulfur analog of an alcohol. In some embodiments, a thiol group may include an organosulfur compound, for example, one that contains a carbon-bonded sulfhydryl. Exemplary thiol groups include —SH, —C—SH, and R-SH, where R represents an organic substituent, e.g., an aryl or alkyl.

As used herein “amino” refers to a compound that contains a nitrogen atom, for example, with a lone pair, attached to a hydrogen atom, alkyl group, or aryl group. In some embodiments, the amino may be derived from ammonia, for example, wherein one or more hydrogen atoms have been replaced by a substituent, for example an aryl or alkyl. The amino may be organic or inorganic. In some embodiments, an amino includes —NH₂, an amino acid, a biogenic amine, trimethylamine, and aniline.

### Compositions

The present disclosure features compositions described herein including adhesive compositions comprising a mixture of a multivalent metal salt, an multidentate acidic organic compound, a carbonate salt, and an aqueous medium, e.g., water, water solution, water suspension, colloid, or aqueous hydrogel. In some embodiments, the compositions described herein further include an additive. In some embodiments the additive is a monovalent metal salt. In some embodiments the additive is an organic or inorganic acid. In some embodiments, the additive is a porogen. In some embodiments, the additive is a pH adjusting agent. In some embodiments the additive is a conjugate salt of a weak acid.

Exemplary multivalent metal salts may include calcium phosphates (e.g., hydroxyapatite, octacalcium phosphate, tetracalcium phosphate, tricalcium phosphate), calcium citrate, sodium citrate, calcium carbonate, magnesium phosphates, magnesium citrate, magnesium carbonate, magnesium hydroxide, strontium citrate, sodium silicates, alkaline calcium silicates, lithium phosphates, titanium phosphates, strontium phosphates, strontium carbonate, strontium oxide, barium sulfate, zinc phosphates, calcium oxide, magnesium oxide, calcium silicate, and combinations thereof.

The amount of each multivalent metal salt (e.g., a calcium phosphate, a calcium oxide, or a combination thereof) may vary, e.g., between about 10% to about 90% weight by weight (w/w) of the total composition. In some embodiments, the amount of the multivalent metal salt (e.g., a calcium phosphate or calcium oxide or a combination thereof) is in the range of about 10% to about 90%, about 15% to about 85%, about 20% to about 80%, about 30% to about 75%, about 40% to about 70%, or about 50% to about 65% w/w of the total composition. In other embodiments, the amount of the metal salt (e.g., a calcium phosphate or calcium oxide or a combination thereof) is in the range of about 5% to about 95%, about 10% to about 85%, about 15% to about 75%, about 20% to about 65%, about 25% to about 55%, or about 35% to about 50% w/w of the total composition.

In some embodiments, the multivalent metal salt comprises one or more alkaline earth metals, e.g., beryllium, magnesium, barium, radium, strontium, or calcium. In some embodiments, the multivalent metal salt may comprise a mixed salt of several metal ions, e.g., dolomite or another mixed salt of alkali earth metal ions. In some embodiments, the multivalent metal salt comprises calcium. In some embodiments, the multivalent metal salt comprises calcium and phosphate. In some embodiments, the multivalent metal salt comprises tetracalcium phosphate. In some embodiments, the composition comprises a plurality of multivalent metal salt compounds. In some embodiments, the plurality comprises tetracalcium phosphate and at least one other multivalent metal salt compound. In some embodiments, the multivalent metal salt comprises hydroxyapatite. In some embodiments, the multivalent metal salts comprise tricalcium phosphate. In some embodiments, the tricalcium phosphate comprises either alpha tricalcium phosphate or beta tricalcium phosphate. In some embodiments, the multivalent metal salts comprise an oxide. In some embodiments, the multivalent metal salt is calcium oxide. In some embodiments, the multivalent metal salt is magnesium oxide. In some embodiments, the multivalent metal salt compound does not comprise tetra-calcium phosphate. In some embodiments, the composition comprises tricalcium phosphate and calcium oxide. In some embodiments, the multivalent metal salt is calcium silicate.

In some embodiments the adhesive composition comprises silicate salts among the constituents of the solid multivalent metal salts. In some embodiments of the adhesive composition these silicate salts are silicates of Group 1 and Group 2 elements. In some embodiments of the adhesive composition these silicate salts are alkaline. In some embodiments of the adhesive composition these silicate salts are sodium metasilicate, sodium orthosilicate, calcium metasilicate, calcium orthosilicate, or a combination thereof. In some embodiments a saturated solution of these silicate salts may exhibit a pH value between 7.4 and 9.95. In some embodiments of the adhesive composition these silicate salts are calcium metasilicate, calcium oxide content of 65% w/w; or calcium orthosilicate, calcium oxide content 48% w/w; or a combination thereof. In some embodiments of the adhesive composition the calcium silicate salts may comprise calcium oxide content in the range between 20 and 65% w/w of the calcium silicate total dry mass.

In some embodiments the adhesive composition comprises calcium silicate among the constituents of the solid multivalent metal salts. The amount of calcium silicate multivalent metal salt (e.g., a calcium metasilicate, calcium orthophosphate, or a combination thereof) may vary, e.g., between about 0.25% to about 75% weight by weight (w/w) of the total multivalent metal salts mass. In some embodiments, the amount of the silicate multivalent metal salt (e.g., calcium metasilicate or calcium orthosilicate, or a combination thereof) is in the range of about 0.25% to about 1.0%, about 1% to about 2%, about 1.5% to about 4%, about 2% to about 6%, about 3% to about 8%, about 5% to about 12%, about 7% to about 14%, about 9% to about 16%, about 11% to about 20%, about 12% to about 22%, about 15% to about 25%, about 18% to about 27%, about 21% to about 30%, about 25% to about 35%, about 30% to about 40%, about 35% to about 45%, about 40% to about 50%, about 45% to about 55%, about 50% to about 60%, about 55% to about 65%, about 60% to about 70%, or about 65% to about 75% w/w of the total multivalent salts mass of the adhesive composition.

In some embodiments, the multivalent metal salt is initially provided as a powder or as a granule. These powders may exhibit a mean particle size of about 0.0001 to about 1.000 mm, about 0.0005 to about 0.001 mm, about 0.001 to about 0.025 mm, about 0.005 to about 0.015 mm, about 0.001 to about 1.000 mm, about 0.001 to about 0.250 mm, about 0.005 to about 0.150 mm, about 0.250 to about 0.750 mm, 0.25 to about 0.50 mm, 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm. The powder may have a mean particle size of less than about 1.000 mm. In some embodiments, the multivalent metal powders may exhibit a minimum percentage of mass content of particles with particle size less than or equal to 0.090 mm, more specifically particles less than or equal to 0.045 mm, wherein the minimum percentage is at least 10 percentage. The particle size distribution may be multi-modal to include any combination of mean particle sizes as previously described. These granules may exhibit a mean granule size of about 0.0125 mm to about 10 mm, about 0.025 mm to about 7.5 mm, about 0.050 mm to about 5 mm, about 0.075 mm to about 2.5 mm, about 0.100 to about 2 mm, about 0.100 to about 1.500 mm, about 0.125 to about 1.000 mm, about 0.125 to about 0.500 mm, about 0.125 mm to about 0.375 mm, about 0.125 to about 0.250 mm, about 0.150 mm to about 0.250 mm, about 0.175 mm to about 0.250 mm, about 0.250 to about 0.750 mm, about 0.250 to about 0.500 mm, about 0.500 to about 1.00 mm, about 0.500 to about 0.750 mm. The granule size distribution may be multi-modal to include any combination of mean granule sizes as previously described. In some embodiments, the granules may be supplied with a various proportion of porosity and a various size of internal pores. The pores may communicate with each other. The pores may communicate with granule surface. In some embodiments, the pores do not communicate with each other. In some embodiments, the pores do not communicate with granule surface. In some embodiments, varying sizes of said powders or granules may be used in the adhesive composition.

In some embodiments, the multidentate acidic organic compound (e.g., phosphoserine) may react with the multivalent metal salts (e.g., tetracalcium phosphate, tricalcium phosphate, calcium oxide, calcium citrate, magnesium carbonate, etc.) to form an adhesive composition when combined with an aqueous medium. The term “multidentate acidic organic compound” as used herein is synonymous with the term “organic phosphate compound” also used herein. The multidentate acidic organic compound may be described by a compound of Formula (I) or a salt thereof:

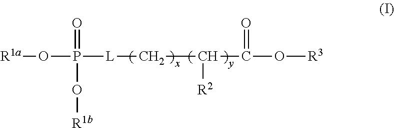

wherein L is O, S, NH, or CH₂; each of R^{1a} and R^{1b} is independently H, optionally substituted alkyl, or optionally substituted aryl; R² is H, NR^{4a}R^{4b}, C(O)R⁵, or C(O)OR⁵; R³ is H, optionally substituted alkyl, or optionally substituted aryl; each of R^{4a} and R^{4a} is independently H, C(O)R⁶, or optionally substituted alkyl; R^{S} is H, optionally substituted alkyl, or optionally substituted aryl; R⁶ is optionally substituted alkyl or optionally substituted aryl; and each of x and y is independently 0, 1, 2, or 3.

In some embodiments, L is 0 or S. In some embodiments, L is 0. In some embodiments, each of R^{1a} and R^{1b} is independently H. In some embodiments, L is 0 and each of R^{1a} and R^{1b} is independently H. In some embodiments, R² is H, NR^{4a}R^{4b}, or C(O)R⁵. In some embodiments, R² is NR^{4a}R^{4b}. In some embodiments, R² is NR^{4a}R^{4b} and each of R^{4a} and R^{4b} is independently H. In some embodiments, L is 0, each of R^{1a} and R^{1b} is H, R² is NR^{4a}R^{4b}, and each of R^{4a} and R^{4b} is independently H. In some embodiments, R³ is H. In some embodiments, L is 0, each of R^{1a} and R^{1b} is independently H, R² is NR^{4a}R^{4b}, each of R^{4a} and R^{4b} is independently H, and R³ is H. In some embodiments, each of x and y is 0 or 1. In some embodiments, each of x and y is 1.

In some embodiments, L is 0, each of R^{1a} and R^{1b} is H, R² is NR^{4a}R^{4b}, each of R^{4a} and R^{4b} is independently H, R³ is H, and each of x and y is 1. In some embodiments, the multidentate acidic organic compound (e.g., a compound of Formula (I)) is phosphoserine.

As used herein, the term “optionally substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds (e.g., alkyl, alkenyl, alkynyl, cycloalkyl, heterocycloalkyl, aryl, or heteroaryl, any of which may itself be further substituted), as well as halogen, carbonyl (e.g., aldehyde, ketone, ester, carboxyl, or formyl), thiocarbonyl (e.g., thioester, thiocarboxylate, or thioformate), amino, —N(R^{b})(R^{c}), wherein each R^{b} and R^{c} is independently H or C₁-C₆ alkyl, cyano, nitro, —SO₂N(R^{b})(R′), —SOR^{d}, and S(O)₂R^{d}, wherein each R^{b}, R^{c}, and R^{d} is independently H or C₁-C₆ alkyl. Illustrative substituents include, for example, those described herein above. The permissible substituents can be one or more and the same or different for appropriate organic compounds. This disclosure is not intended to be limited in any manner by the permissible substituents of organic compounds. It will be further understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

In some embodiments, the molecular weight of the multidentate acidic organic compound is below about 1000 g/mol. In some embodiments, the molecular weight of the multidentate acidic organic compound is between about 150 g/mol and about 1000 g/mol, e.g., between about 155 g/mol and about 750 g/mol, between about 160 g/mol and about 500 g/mol, between about 165 g/mol and about 250 g/mol, between about 170 g/mol and about 200 g/mol, or between about 175 g/mol and about 190 g/mol. In some embodiments, the molecular weight the multidentate acidic organic compound is between about 180 g/mol and about 190 g/mol.

The multidentate acidic organic compound of Formula (I) may adopt any stereoisomeric form or contain a mixture of stereoisomers. For example, the multidentate acidic organic compound may be a mixture of D,L-phosphoserine, or contain substantially pure D-phosphoserine or substantially pure L-phosphoserine. In many embodiments, the stereochemistry of the multidentate acidic organic compound does not significantly impact the physical (e.g., adhesive) or regeneration properties of the composition. In some embodiments, the particular stereochemistry of the organic phosphate or the ratio of stereoisomers of the multidentate acidic organic compound has a significant impact on the regeneration properties of the composition.

In some embodiments, the multidentate acidic organic compound (e.g., a compound of Formula (I)) is present in an amount greater than or equal to about 0.1% (w/w) of the composition. In some embodiments, the multidentate acidic organic compound (e.g., a compound of Formula (I)) is present in an amount greater than or equal to about 0.1% (w/w), about 0.5% (w/w), about 1% (w/w), about 3% (w/w), about 5% (w/w), about 10% (w/w), about 20% (w/w), about 30% (w/w), about 40% (w/w), about 50% (w/w), about 60% (w/w), about 70% (w/w), about 80% (w/w), about 90% (w/w), about 95% (w/w), or up to 100% of the composition.

In some embodiments, the adhesive composition may further comprise a porogen. As used herein, the term “porogen” refers to a particle of a defined shape or a defined volume, which may, in an embodiment, be cured within a set structure. The porogen may leave void spaces where the porogen was embedded after curing. Exemplary porogens include carbonate salts of alkaline metal elements (e.g., lithium, sodium, potassium) and alkaline earth elements (beryllium, magnesium, calcium, strontium). A porogen may be used in the adhesive compositions disclosed herein to create a plurality of pores within the substance of the composition during curing. In an embodiment, the porogen comprises a calcium salt, e.g., calcium carbonate.

It has been observed that the pH of the adhesive composition mixture at the time of activation by combining the powdered and liquid precursors and mixing may unphysiologically low (pH ˜5). In an embodiment, in these scenarios, the pH returns to physiologically appropriate levels later during the curing process (pH ˜7). However, the passing stage of unphysiologically low pH, i.e., high acidity, has been observed to be cytotoxic to the tissues surrounding the composition at that phase of curing.

In some embodiments, the porosity of the adhesive composition, upon curing, is between 10% to 80%, e.g., between 10% to 50%, between 50% to 80%, between 12% to 48%, between 14% to 46%, between 16% to 44%, between 18% to 42%, between 20% to 40%, between 22% to 38%, between 24% to 36%, between 26% to 34%, between 28% to 32%, or about 30%, e.g., between 10% to 30%, between 15% to 35%, between 20% to 40%, between 25% to 45%, or between 30% to 50%, e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%.

In some embodiments, the plurality of pores of the cured adhesive composition can have a pore size of between 0.005 m to 1000 m, e.g., between 0.005 m to 0.05 m, between 0.05 μm to 0.1, between 0.1 μm to 1.0 μm, between 1.0 μm to 10 am, between 10 m to 20 μm, between 20 μm to 200 μm, between 25 μm to 190 μm, between 30 μm to 180 μm, between 35 μm to 170 μm, between 40 μm to 160 μm, between 45 μm to 150 μm, between 50 μm to 140 μm, between 55 μm to 130 μm, between 60 μm to 120 μm, between 65 μm to 110 μm, between 70 μm to 100 μm, or between 80 μm to 90 μm, e.g., between 20 μm to 50 μm, between 30 μm to 60 μm, between 50 μm to 90 m, between 75 m to 100 m, between 90 μm to 120 μm, between 100 μm to 150 μm, between 120 μm to 170 μm, or between 150 μm to 200 μm, e.g., about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, or 200 μm. In some embodiments, the plurality of pores of the cured adhesive composition can be multimodal, including a combination of any of the pore size ranges listed above.

In some embodiments, the adhesive composition, upon curing, can have a density between 0.75 g/cm³ to 1.40 g/cm³, e.g., between 0.75 g/cm³ to 1.40 g/cm³, between 0.80 g/cm³ to 1.35 g/cm³, between 0.85 g/cm³ to 1.30 g/cm³, between 0.90 g/cm³ to 1.25 g/cm³, between 0.95 g/cm³ to 1.20 g/cm³, between 1.00 g/cm³ to 1.15 g/cm³, or between 1.05 g/cm³ to 1.10 g/cm³, e.g., between 0.75 g/cm³ to 1.00 g/cm³, between 0.80 g/cm³ to 1.05 g/cm³, between 0.85 g/cm³ to 1.10 g/cm³, between 0.90 g/cm³ to 1.15 g/cm³, between 0.95 g/cm³ to 1.20 g/cm³, between 1.00 g/cm³ to 1.25 g/cm³, between 1.05 g/cm³ to 1.30 g/cm³, between 1.10 g/cm³ to 1.35 g/cm³, or between 1.15 g/cm³ to 1.40 g/cm³, e.g., about 0.75 g/cm³, about 0.80 g/cm³, about 0.85 g/cm³, about 0.90 g/cm³, about 0.95 g/cm³, about 1.00 g/cm³, about 1.05 g/cm³, about 1.10 g/cm³, about 1.15 g/cm³, about 1.20 g/cm³, about 1.25 g/cm³, about 1.30 g/cm³, about 1.35 g/cm³, or about 1.40 g/cm³.

In some embodiments, the aqueous medium comprises water (e.g., sterile water), saliva, buffers (e.g., sodium phosphate, potassium phosphate, or saline (e.g., phosphate buffered saline)), pH adjusting agents, blood, blood-based solutions (e.g., plasma, serum, bone marrow), spinal fluid, dental pulp, cell-based solutions (e.g., solutions comprising fibroblasts, osteoblasts, platelets, odontoblasts, stem cells (e.g., mesenchymal stem cells) histiocytes, macrophages, mast cells, or plasma cells), or combinations thereof in the form of aqueous solutions, suspensions, and colloids. In some embodiments, the aqueous medium comprises sterile water, distilled water, deionized water, sea water, or fresh water.

In some embodiments, a pH of the active reaction mixture of the activated composition is generally acidic, i.e., pH<7. This acidity causes the immediate vicinity of the implanted adhesive composition following application to become acidic as well. Even though over hours to days the pH rises towards neutral, becomes neutral, or becomes slightly alkaline, e.g., a pH of about 7.5-8.5, as the adhesive composition cures. Without wishing to be bound by any particular theory, the solubility of the acidic component of the adhesive composition, e.g., the multidentate acidic organic compound, is approximately 300,000× greater than the solubility of the alkaline component on molarity basis at neutral pH, e.g., pH of 7, suggesting that inclusion of dissolved or readily soluble alkali compounds to increase the pH of the surrounding milieu may modulate the initial drop in pH to less unphysiological levels. While in the tacky state, the pH of the adhesive composition is between 6 to 9.5, e.g., the pH is 6, 6.5, 7, 7.5, 8, 8.5, or 9.

In some embodiments, the adhesive composition may include an acidity adjusting agent. As disclosed herein, the pH of the adhesive composition might in some embodiments comprising the multidentate organic acidic Compound of Formula I (phosphoserine) and the alkaline multivalent metal salt, MVMS, which comprises mixed alkaline calcium phosphates, i.e., tetracalcium phosphate (TTCP), α-tricalcium phosphate (α-TCP), and hydroxyapatite (HA), is acidic immediately following activation, i.e., mixing of the powdered precursors and the aqueous medium, as a result of differential dissolution rate of the acidic component being much higher than that of the alkaline component, reaching pH values of 5.3, or lower. This transient decrease in pH may be followed by a return to neutral pH upon curing; however, the high acidity of this implantable material can damage host tissues, resulting in high cytotoxicity and low survival rates (20%) of neighboring cells. The acidity adjusting agent can be any suitable compound that can be used to adjust the pH a desired amount. In certain embodiments, the acidity adjusting agent is a basic acidity adjusting agent, e.g., an agent with a pH greater than 7. The basic acidity adjusting agent can be any aqueous soluble basic compound including, but not limited to, the oxide and hydroxide salts of the Group I or Group II elements, e.g., alkali metal hydroxide, an alkali metal oxide, an alkaline earth metal hydroxide, an alkaline earth metal oxide, or a combination thereof. For example, the basic acidity adjusting agent can be selected from the group consisting of sodium hydroxide, potassium hydroxide, lithium hydroxide, magnesium hydroxide, or a combination thereof. In particular embodiments, the basic acidity adjusting agent is sodium hydroxide. In some embodiments, the aqueous medium contains the acidity adjusting agent. In specific embodiments, the aqueous medium is sodium hydroxide. In some embodiments of any and all aspects of the present disclosure, the acidity adjusting agent is sodium hydroxide that has a concentration between 0.5 and 5 M.

It is also envisioned that the relationship between the porogen and the pH modulating influence of the additives to the composition can be controlled by their specific selection. For example, the series of salts, e.g., an oxide, a hydroxide, a carbonate, and a bicarbonate of the same element, e.g., calcium, when used as additives to the powdered components of the system, will generate different, yet progressive, effects on both porosity and acidity. Calcium oxide, CaO, and its solubilized form, calcium hydroxide, Ca(OH)₂, being the most alkaline (pKa=10.9), will have stronger influence on the pH than the carbonates, but do not increase the porosity of the composition because no gas is released during neutralization. Calcium carbonate, CaCO₃, is both alkaline (pKa=10.3) and a porogen, capable of releasing one equivalent of carbon dioxide per mole. Calcium bicarbonate, Ca(HCO₃)₂, being the least alkaline (pKa=8.3) of the series, will reduce the acidity the least; however, it will increase porosity the most because it can release two equivalents of carbon dioxide per mole.

According to one embodiment, the additive is calcium oxide, CaO, which, in presence of water becomes hydrated to produce calcium hydroxide, Ca(OH)₂, and dissolves according to Equation 1.

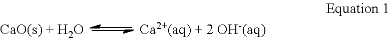

In presence of acid the two hydroxide ions, OH⁻(aq), become protonated with two H⁺ ions to produce two water molecules, thus neutralizing the acid present, as shown in Equation 2.

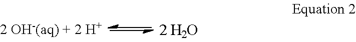

One significant net result of the sequence of events is the consumption of two hydrogen ion equivalents (i.e., acid), generating a less acidic environment by reduction of hydrogen ion concentration. In an embodiment, no porosity is generated as a result.

According to another embodiment of the invention, the additive is calcium carbonate, which is water soluble according to Equation 3.

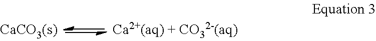

When exposed to acid, the dissolved carbonate ion, CO₃²⁻(aq), is converted through protonation to carbonic acid, H₂CO₃(aq) according to Equation 4, thereby consuming acidity.

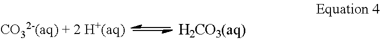

Carbonic acid dissociates into carbon dioxide, CO₂(aq), and water, as shown in Equation 5. The dissolved carbon dioxide is liberated into the gas phase and is eliminated from the reaction as its concentration rises.

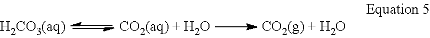

One significant net result of this sequence of events is the consumption two hydrogen ion equivalents (i.e., acid), generating a less acidic environment by reduction of hydrogen ion concentration, and the release of one carbon dioxide gas equivalent within the composition.

According to yet another embodiment of the invention, the additive is calcium bicarbonate, Ca(HCO₃)₂(s), which is a water soluble solid according to Equation 6.

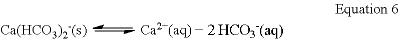

When exposed to acid, the dissolved bicarbonate ions, HCO₃⁻(aq), are converted through protonation to carbonic acid, H₂CO₃(aq), according to Equation 7, thereby consuming acidity.

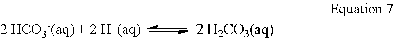

Carbonic acid dissociates into carbon dioxide, CO_{2(aq)}, and water, as shown in Equation 8. The dissolved carbon dioxide is liberated into the gas phase and is eliminated from the reaction as its concentration rises.

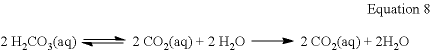

One significant net result of this sequence of events is the consumption of two hydrogen ion equivalents (i.e., acid), generating a less acidic environment by reduction of hydrogen ion concentration, and the release of two carbon dioxide gas equivalents within the composition. In some embodiments of the invention, a carbonate, a bicarbonate, or a combination of both, may be used to optimize the porosity content of the composition and the acidity content of the composition independently of one another by taking advantage of these differences. The gas is released as bubbles within the substance of the reaction mixture. These carbon dioxide gas bubbles may become pores within the composition as it cures into a solid substance. Following implantation, and, for example, at a much slower rate, the carbon dioxide may be lost from the site and substituted by liquid water from the extracellular fluid environment.

Given the initial low pH of the adhesive composition reaction mixture as the material is applied to the tissues of the host, another method of modulating the initial acidity of the composition with is hereby disclosed. Without wishing to be bound by any particular theory, the acidity of the active reaction of the composition may be related to the dissolution and dissociation of an acidic species, e.g., compound of Formula I. Supplying a dissolved alkaline additive substance with capacity to neutralize the acid within the aqueous medium may provide a kinetic advantage in modulating the unphysiologically high acidity at the time of implantation of the adhesive composition. Such additives must fulfill the requirements that they are sufficiently alkaline and sufficiently soluble in the aqueous medium, e.g., compounds including the conjugate bases of weak acids, particularly polyprotic acids, and be present as soluble salts. Soluble salts may include salts of alkaline metal ions, e.g., sodium, potassium, which are present in the host tissues in sufficiently high concentrations not to perturb the homeostatic balance. Example of salts which are both highly soluble and not toxic as used in the system are sodium hydroxide, which generates water when interacting with the acidic hydrogen ion, and tribasic sodium citrate, which is a good buffer in the pH range of interest, a strong chelator of calcium contributing to hardening of the composition, and a compound readily metabolized by the host.

In some embodiments, the compositions further comprise an additive or a plurality of additives. An additive may be used to impart additional functionality to the composition of the disclosure, such as improving or affecting the handling, texture, durability, strength, multidentate acidic organic compound release, or resorption rate of the material, or to provide additional cosmetic or medical properties. Exemplary additives may include salts (e.g., sodium bicarbonate, sodium chloride, sodium phosphate, sodium hydroxide, potassium chloride), polymers, fillers or physical modifiers (e.g., granules or fibers), activity modifiers (e.g., adsorption agents), formulation bases, viscosity modifiers (e.g., polyols (e.g., glycerol, mannitol, sorbitol, trehalose, lactose, glucose, fructose, or sucrose)), bone fragments, bone chips, coloring agents (e.g., dyes or pigments), flavoring agents (e.g., sweeteners), medications that act locally (e.g., anesthetics, coagulants, clotting factors, chemotactic agents, agents inducing phenotypic change in local cells or tissues, and signaling system components or modifiers), medications that act systemically (e.g., analgesics, anticoagulants, hormones, vitamins, pain relievers, anti-inflammatory agents), antimicrobial agents (e.g., antibacterial, antiviral, or antifungal agents) or combinations thereof. The biologically active substances (e.g., medicines) in the categories above might include active substances or precursors, which become biologically active upon modification after interaction with the surrounding environment. The substances might be synthetic, semisynthetic, or biologically derived (e.g., peptides, proteins (e.g., bone morphogenetic protein), or small molecules). The substances might include, but not be limited to anti-inflammatories (e.g., steroids, nonsteroidal anti-inflammatory drugs, cyclooxygenase inhibitors), complement proteins, bone morphogenic factors and proteins, hormones active locally or systemically (e.g., parathyroid hormone, calcitocin, prostaglandins), or other small molecules (e.g., calciferols).

In some embodiments, the additive is a polymer. These polymeric based compounds may include one or more of a poly(L-lactide), poly(D,L-lactide), polyglycolide, poly(α-caprolactone), poly(teramethylglycolic-acid), poly(dioxanone), poly(hydroxybutyrate), poly(hydroxyvalerate), poly(lactide-co-glycolide), poly(glycolide-co-trimethylene carbonate), poly(glycolide-co-caprolactone), poly(glycolide-co-dioxanone-co-trimethylene-carbonate), poly(tetramethylglycolic-acid-co-dioxanone-co-trimethylenecarbonate), poly(glycolide-co-caprolactone-co-lactide-co-trimethylene-carbonate), poly(hydroxybutyrate-co-hydroxyvalerate), poly(methylmethacrylate), poly(acrylate), a polyamine, a polyamide, a polyimidazole, poly(vinyl-pyrrolidone), collagen, silk, chitosan, hyaluronic acid, collagen, gelatin and/or mixtures thereof. In addition, co-polymers of the above homopolymers also can be used.

In some embodiments, the fillers or physical modifiers are made from tricalcium phosphate (in either the alpha or beta form), hydroxyapatite, or mixtures thereof. The fillers or physical modifiers may also be made from biodegradable polymers such as polyethylene glycol (PEG), polylactic acid (PLLA), polyglycolic acid (PGA), and copolymers of lactic and glycolic acid (PLGA) and may further comprise biodegradable block polymers such as polylactic acid (PLLA)-polyethylene glycol (PEG)-polylactic acid (PLLA) block polymer.

In some embodiments, the adhesive composition comprises some or all of the alkaline multivalent metal salt solid in granulated or powder form, or any combination thereof. These particulate solids may exhibit a mean particle size of about 0.0005 to about 0.500 mm, about 0.001 to about 0.250 mm, about 0.005 to about 0.150 mm, about 0.0075 to about 0.100 mm, about 0.01 to about 0.025 mm, about 0.020 to about 0.060 mm, about 0.020 to about 0.040 mm, about 0.040 to about 0.100 mm, about 0.040 to about 0.060 mm, about 0.050 to about 0.100 mm about 0.050 to about 0.150 mm, about 0.060 to about 0.150 mm, about 0.060 to about 0.125 mm, about 0.075 to about 0.150 mm, or about 0.075 to about 0.125 mm. The mean particle size may be bimodal to include any combination of mean particle sizes as previously described. These granules may exhibit a mean granule size of about 0.0125 mm to about 10 mm, about 0.025 mm to about 7.5 mm, about 0.050 mm to about 5 mm, about 0.075 mm to about 2.5 mm, about 0.100 to about 2 mm, about 0.100 to about 1.500 mm, about 0.125 to about 1.000 mm, about 0.125 to about 0.500 mm, about 0.125 mm to about 0.375 mm, about 0.125 to about 0.250 mm, about 0.150 mm to about 0.250 mm, about 0.175 mm to about 0.250 mm, about 0.250 to about 0.750 mm, about 0.250 to about 0.500 mm, about 0.500 to about 1.00 mm, about 0.500 to about 0.750 mm. The mean granule size may be multi-modal to include any combination of mean granule sizes as previously described. In some embodiments, varying sizes of said powders or granules may be used in the adhesive composition. In some embodiments the size of the multivalent metal salt particles may be selected to provide control over specific composition characteristics or behaviors. In some embodiments the size of the multivalent metal salt particles may be selected to provide control over the rate of the setting reaction. In some embodiments the size of the multivalent metal salt particles may be selected to provide control over the rate of the pH of the reaction mixture, e.g., by controlling the surface area of alkaline multivalent metal salt constituent during the early phase of the reaction. In some embodiments the size of the multivalent metal salt particles may be selected to provide control over the mechanical properties of the material, e.g., viscosity of the injectable composition, compressive strength of the composition, adhesive properties of the composition, and the like.

In some embodiments, certain additives may be provided as powders or granules or solutes or any combination thereof. These powders may exhibit a mean particle size of about 0.0005 to about 0.500 mm, about 0.001 to about 0.250 mm, about 0.005 to about 0.150 mm, about 0.0075 to about 0.100 mm, about 0.01 to about 0.025 mm, about 0.020 to about 0.060 mm, about 0.020 to about 0.040 mm, about 0.040 to about 0.100 mm, about 0.040 to about 0.060 mm, about 0.050 to about 0.100 mm about 0.050 to about 0.150 mm, about 0.060 to about 0.150 mm, about 0.060 to about 0.125 mm, about 0.075 to about 0.150 mm, or about 0.075 to about 0.125 mm. The mean particle size may be bimodal to include any combination of mean particle sizes as previously described. These granules may exhibit a mean granule size of about 0.0125 mm to about 10 mm, about 0.025 mm to about 7.5 mm, about 0.050 mm to about 5 mm, about 0.075 mm to about 2.5 mm, about 0.100 to about 2 mm, about 0.100 to about 1.500 mm, about 0.125 to about 1.000 mm, about 0.125 to about 0.500 mm, about 0.125 mm to about 0.375 mm, about 0.125 to about 0.250 mm, about 0.150 mm to about 0.250 mm, about 0.175 mm to about 0.250 mm, about 0.250 to about 0.750 mm, about 0.250 to about 0.500 mm, about 0.500 to about 1.00 mm, about 0.500 to about 0.750 mm. The mean granule size may be multi-modal to include any combination of mean granule sizes as previously described. In some embodiments, varying sizes of said powders or granules may be used in the adhesive composition. In some embodiments the size of the additive particles may be selected to provide control over the rate of the setting reaction. In some embodiments the size of the additive particles may be selected to provide control over the rate of the pH of the reaction mixture, e.g., by controlling the surface area of alkaline additive during the early phase of the reaction. In some embodiments the size of the additive particles may be selected to provide control over the mechanical properties of the material, e.g., viscosity of the injectable composition, compressive strength of the composition, adhesive properties of the composition, etc.

In some embodiments the specifics of the particle size distribution of these granules are selected to produce intergranular spaces and passages of desired magnitude, this magnitude generally referring to the diameter of a hypothetical sphere capable of passing through stacked granule space. In some embodiments the magnitude of the intergranular passages is from about 50 micron to about 500 micron, e.g., about 50 micron to about 500 micron, about 75 micron to about 475 micron, about 100 micron to about 450 micron, about 125 micron to about 425 micron, about 150 micron to about 400 micron, about 175 micron to about 375 micron, about 200 micron to about 350 micron, about 225 micron to about 325 micron, or about 250 micron to about 300 micron. In some embodiments, the magnitude of the intergranular passages is greater than about 100 microns but less than 400 microns. In some embodiments the magnitude of the intergranular spaces and passages depends on the general shape of the granules forming the scaffold for the same spaces and passages. In some embodiments the intergranular passages facilitate exchange of tissue fluids through the bulk of the composition. In some embodiments the intergranular passages facilitate dissolution of the composition in tissue fluids. In some embodiments the intergranular passages facilitate neovascularization of the bulk of the composition. In some embodiments the intergranular passages facilitate ingrowth of host tissues into the bulk of the composition. In some embodiments the intergranular passages facilitate replacement of the composition substance by bone. In some embodiments the intergranular passages facilitate dispersal out of the composition and into surrounding tissues of the organic acidic compound constituent of the adhesive composition, e.g., phosphoserine or its conjugate bases. In some embodiments, the intergranular passages facilitate dispersal out of the composition and into surrounding tissues of additives diffusing. In some embodiments the compounds diffusing out of the composition through the intergranular passages may provide therapeutic effects, e.g., stimulation of bone repair, pain control, antimicrobial activity, hormonal activity, and the like.

In some embodiments, the adhesive composition can be applied to the surface of a structure in its fluid or semi-solid state by means of an injection delivery device or by application using an instrument such as a spatula. The viscosity of the adhesive composition when in its fluid state might be as low as about 100 cP to about 10,000 cP, e.g., about 100 cP to about 500 cP, about 500 cP to about 1,000 cP, about 1,000 cP to about 2,000 cP, about 2,000 cP to about 5,000 cP, about 5,000 cP to about 7,000 cP, or about 7,000 cP to about 10,000 cP. The viscosity of the adhesive composition when in its semi-solid state may range from about 10,000 cP to about 350,000 cP. In some embodiments, the viscosity and cohesion properties of the composition may facilitate the ability to squeeze the material through a needle or cannula as small as 18 gauge when the viscosity is in the low range of its fluid state. In some embodiments, with viscosities in the semi-solid state, the shape and amount of material can be altered through smearing, spreading, molding, or removal techniques without substantially effecting the strength of the set material.

In some embodiments, the adhesive compositions are injectable and their injectability can be controlled by selection of particle size of the multivalent metal salts. The injectability of the adhesive composition can be assessed by the amount of force required to dispense the adhesive composition through a clinically relevant hand actuated delivery device, e.g., a syringe, cannula.

In some embodiments the multivalent metal salts comprise particles which are less than or equal to a critical size and are present in sufficient mass fraction to improve flowability and reduce the piston force required for the injection. In some embodiments the multivalent metal salts particles comprise fines within the particle population. Fines are herein defined as a particle population in which ninety percent of the volume, i.e., d90, is constituted by particles less than or equal to 0.045 mm in diameter as measured by laser diffraction method. In some embodiments the adhesive composition comprises at least ten percent w/w fines of the multivalent metal salts constituent to reduce the injection force. Without wishing to be bound by any particular theory, the fines fraction of the active reaction mixture presents a disproportionately large surface area to the acidic solution of the multidentate organic acid compound surrounding it and is consequently disproportionately reduced in mass by etching away. The fines granules, therefore, are either eliminated outright or reduced in diameter. Both outcomes favor lower viscosity, the former by eliminating solids which disrupt fluid flow, and the latter by increasing the lubricity of the mixture by increasing the influence of adsorbed and coordinated small molecules on friction between solid particles.

In some embodiments, the adhesive composition may have a tacky state after mixing with an aqueous medium. In some embodiments, this tacky property is retained for a number seconds (e.g., up to 30 seconds, up to 5 seconds, up to 2 seconds), up to minutes (e.g., up to 30 minutes, up to 12 minutes, up to about 4 minutes, up to about 2 minutes, up to about 1 minute), up to hours (e.g., up to 12 hours, up to 4 hours, up to 1 hour), up to days (e.g., up to 7 days, up to 3 days, up to 1 day), after mixing with the aqueous medium. The duration of the tacky state may be dependent on a number of factors including relative ratio of the components, the particle sizes of the component materials, the presence of additives and the like, or the temperature of the environment. In some embodiments, during the tacky state, the adhesive composition will adhere to surfaces without the need for external clamping or other application of pressure. In some embodiments, the adhesive composition in the tacky state will adhere bone to bone and bone to other materials. In some embodiments, the adhesive composition in the tacky state may adhere materials such as steel, e.g., stainless steel, titanium, zirconia, polyether ether ketone, aluminum, copper, brass, aragonite, calcite, cement, alumina, concrete, ceramics, rock, glass, and other metals or substances. In some embodiments, during the tacky state the contacting surfaces may be held together by the adhesive composition itself, without the need for external force, until the composition sets to the final hardened cement state. In some embodiments, the tacky state can allow the materials to be positioned or repositioned without appreciable loss of cured strength.

The amount of force needed to separate two adherent pieces of material from each other during the tacky state is the tack strength. In some embodiments, the adhesive composition, when applied to join or affix two surfaces, may have a tack stress, as measured by tensile or shear loads during the tacky state, from about 10 kPa to about 250 kPa and preferably from about 50 kPa to about 150 kPa. In some embodiments, the tack stress may be sufficiently high that the items to be joined need not be held or clamped together unless there is an opposing force, e.g., a separating/tensile force/stress on the surface, inducing stresses greater than the maximum tack stress. During the tacky state the materials may be positioned, repositioned or reopposed several times without appreciable loss of cured adhesive strength.

In some embodiments, the adhesive composition may adopt a pliable working or putty state after mixing with an aqueous medium prior to hardening, which is present for up to about one week or less, one day or less, one hour or less, 30 minutes or less, depending on the components of said adhesive compositions and the conditions of the application, e.g., temperature. In some embodiments, the adhesive composition may adopt a pliable working or putty state for less than or equal to about one week after mixing with an aqueous solution or suspension, e.g., less than about six days, less than about five days, less than about four days, less than about three days, less than about two days, less than about one day, less than about twelve hours, less than about one hour, less than about 30 minutes, less than about 20 minutes, less than about 15 minutes, less than about 10 minutes, less than about 5 minutes, less than about 3 minutes, less than about 2 minutes, less than about 1 minute, less than about 30 seconds, less than about 5 seconds after mixing with an aqueous solution or suspension.

In some embodiments, during the putty state, which follows the tacky state, the adhesive composition can be shaped or sculpted, for example, to fill voids in bone or acquire a desired contour, size or form. The combined time of the tacky state and the putty state is referred to herein as working time. In some embodiments, the adhesive bone regenerative composition may have a working time of up to at least 15 seconds, up to at least 30 seconds, up to at least 1 minute, up to at least 3 minutes, up to at least 5 minutes, up to at least 8 minutes, up to at least 12 minutes, or up to at least 15 minutes from initial mixing, after which time the compositions have sufficiently begun hardening to resist running, sagging, or other spontaneous displacement.

In some embodiments, after a set amount of time, the adhesive composition may adopt a hard, cement-like state. This process of conversion from the pliable working state to the cement-like state may be referred to as “hardening,” “curing,” or “setting.” In some embodiments, the adhesive composition may harden, cure, or set such that the materials that have been affixed to each other with the adhesive compositions cannot be displaced relative one another without the application of significant force. In some embodiments, the adhesive compositions will begin to harden within about 15 minutes, e.g., within about 15 seconds, within about 1 minute, within about 3 minutes within about 5 minutes, or within about 8 minutes, after mixing with the aqueous medium near room or body temperature. In some embodiments, adhesive compositions may be formulated to harden within a specific amount of time. For example, certain formulations may harden within less than 8 minutes, e.g., less than 30 seconds, less than 1 minute, or less than 3 minutes. Other formulations may harden within more than 8 minutes, for example, more than about 12 minutes, more than about 15 minutes, more than one day or about one week. The variance in hardening times may be due to the composition (e.g., additives in the formulation or manufacturing details of the formulation physical state such as powder particle size distribution), the environment (e.g., temperature), and the handling (length of mixing, rate of mixing the composition, and mode of mixing). In some embodiments, hardening time may range between less than 30 seconds to more than one day, under the same external conditions. In some embodiments, the described tacky, putty, and cement-like state can occur in a wet environment or dry environment.

In some embodiments, the adhesive composition may exhibit an adhesive strength in the cement-like state in the range of about 100 kPa to about 12,000 kPa, depending on the application and the particular components and ratios of components in said adhesive compositions. In some embodiments, the adhesive strength of the adhesive composition in the cement-like state is between about 100 kPa and about 10,000 kPa, e.g., about 9,000 kPa, about 8,000 kPa, about 7,000 kPa, about 6,000 kPa, about 5,000 kPa, about 4,000 kPa, about 3,000 kPa, about 2,000 kPa, about 1,000 kPa, about 750 kPa, about 500 kPa, about 250 kPa, or about 200 kPa. In some embodiments, the adhesive strength of the adhesive composition in the cement-like state is between about 100 kPa, about 200 kPa, about 300 kPa, about 400 kPa, about 500 kPa, about 600 kPa, about 700 kPa, about 800 kPa, about 900 kPa, about 1,000 kPa, about 2,500 kPa, about 5,000 kPa, about 7,500 kPa, about 10,000 kPa or about 12,000 kPa. In some embodiments, the adhesive strength of the adhesive composition in the cement-like state is in the range of about 200 kPa and about 2,500 kPa. In some embodiments, the adhesive strength of the adhesive composition in the cement-like state is greater than 100 kPa.

In some embodiments, the adhesive compositions disclosed herein exhibit adhesive behavior toward bone and other materials, including titanium and stainless steel. In some embodiments, the adhesive compositions disclosed herein exhibit bone regenerative behavior, where the substance of the adhesive composition is gradually degraded and replaced with new bone in volume-maintaining manner over time. In some embodiments, the adhesive composition may be degraded and replaced by bone while substantially maintaining the original shape formed during application. In some embodiments, the adhesive composition may be degraded and replaced by bone while substantially maintaining the strength of its attachment to bone as measured by biomechanical testing of objects attached by the composition to bone in vivo, i.e., attachment strength of screw-shaped titanium implants measured by torque required to turn them. In some embodiments, the adhesive composition may be degraded and replaced by bone while substantially maintaining the original shape formed during application. In some embodiments, the adhesive composition may be degraded and replaced by bone while substantially increasing the strength of its attachment to bone as measured by biomechanical testing of objects attached by the composition to bone in vivo, i.e., attachment of screw-shaped titanium implants measured by torque required to turn them. In some embodiments the in vivo degradation proceeds by dissolution of the adhesive composition. In some embodiments the in vivo degradation proceeds by cell-mediated resorption of the adhesive composition. In some embodiments the in vivo degradation proceeds by dissolution and cell-mediated resorption of the adhesive composition.

### Methods of Preparing Adhesive Compositions

In an aspect, there is provided method of preparing an adhesive composition, e.g., an adhesive composition as described herein. The method may include providing a mixture of a multivalent metal salt, multidentate acidic organic compound, and a salt comprising a conjugate base of a weak acid, e.g., carbonate, citrate, phosphate, etc., or an oxide or hydroxide of an alkali element, e.g., Group I or Group II element (e.g., sodium, calcium, magnesium). The method further may include contacting the mixture with an aqueous medium. The method additionally may include adding an acidity adjusting agent to the mixture to reduce the acid content of the mixture to a value of pH between 5.7 to 8, thereby preparing the adhesive composition to exhibit improved biocompatibility.

In an aspect, there is provided method of preparing an adhesive composition, e.g., an adhesive composition as described herein. The method may include providing a mixture of a multivalent metal salt, multidentate acidic organic compound, and a salt comprising a conjugate base of a weak acid, e.g., carbonate or citrate. The method further may include contacting the mixture with an aqueous medium. The method additionally may include adding an acidity adjusting agent to the mixture to evolve a gas from the mixture, thereby preparing the adhesive composition that, upon curing, has a porosity of 10-65%.

In an aspect, there is provided method of preparing an adhesive composition, e.g., an adhesive composition as described herein. The method may include providing a mixture of a multivalent metal salt, multidentate acidic organic compound, and a salt comprising a conjugate base of a weak acid, e.g., carbonate. The method further may include contacting the mixture with an aqueous medium. The method additionally may include adding an acidity adjusting agent to the mixture to evolve a gas from the mixture, thereby preparing the adhesive composition that, upon curing, has a plurality of pores having a pore size of between 20 μm to 200 μm.

In some embodiments, the addition of the pH adjusting agent is used to increase the pH value and to stimulate the formation of a porous adhesive composition. As disclosed herein, the pH of the activated reaction mixture leading to formation of the adhesive composition is initially unphysiologically low. This low pH does not persist, as it is followed by a return to neutral pH upon curing, i.e., within minutes, hours, or days. An acidity reducing agent may be added to the formulation to increase the pH during the working time period of the composition, e.g., during the first minutes or hours following the activation and implantation of the composition to improve the biocompatibility of the adhesive composition. The pH adjusting agent can be any suitable compound that can adjust the pH to a desired level to achieve a desired purpose in the desired time frame. In general, the pH of the mixture used to form the adhesive composition can be adjusted by: 1) adding to the mixture a soluble alkaline ion to balance the decrease in pH as the multidentate acidic organic component is released; 2) adding to the mixture a soluble conjugate base salt of a weak acid to buffer the acidity contributed by the organic phosphate component entering solution.

In some embodiments acidity adjustment can be combined with concomitant and consequent generation of porosity by release of gas within the composition reaction mixture, as follows. Without wishing to be bound by any particular theory, if and when the pH adjusting agent comprises carbonate ion, e.g., calcium carbonate, sodium bicarbonate, magnesium bicarbonate, etc., and the pH of the reaction mixture following its activation is acidic, the protonation of carbonate results in formation of carbonic acid, Equations 4 and 7, which spontaneously dissociates to water and carbon dioxide, as shown in Equations 5 and 8, above.

The carbon dioxide is liberated in the form of gas bubbles within the substance of the composition. Upon curing these bubbles constitute porosity within the solid, cement-like state of the adhesive composition.

In certain embodiments, the pH adjusting agent is a basic pH adjusting agent, e.g., an agent with a pH greater than 7. The basic pH adjusting agent can be any aqueous soluble basic compound including, but not limited to, an alkali metal hydroxide, an alkali metal oxide, an alkaline earth metal hydroxide, an alkaline earth metal oxide, or a combination thereof. For example, the basic pH adjusting agent can be selected from the group consisting of sodium hydroxide, potassium hydroxide, lithium hydroxide, magnesium hydroxide, strontium hydroxide, sodium alkaline silicate or a combination thereof. In particular embodiments, the basic pH adjusting agent is sodium hydroxide. In other embodiments, the pH adjusting agent is a salt of a weak acid, e.g., carbonic acid, citric acid, such as sodium carbonate or a dibasic or tribasic sodium citrate salt. In some embodiments, a basic pH adjusting agent, e.g., sodium hydroxide, can be added to the aqueous medium to, e.g., to increase the pH. Without wishing to be bound by any particular theory, an aqueous medium containing a basic pH adjusting agent, e.g., sodium hydroxide, that is added to a dry mixture of a multivalent metal salt and an multidentate acidic organic compound, counters the pH drop expected from the dissolution of acidic species. This neutralization can raise the lowest levels of pH reached by the reaction mixture during the dissolution of the organic phosphate into the same aqueous medium and thus limit damage to surrounding tissues. Once again, without wishing to be bound by any particular theory, given that the pKa of the multidentate acidic organic compound, e.g., phosphoserine, might be near 5.6-5.8, the pH of the composition is buffered by the multidentate acidic organic compound to that range and does not appreciably rise upon the addition of the basic agent in spite of the total amount of acid requiring to be neutralized by the host tissues being significantly reduced. The reduction of the free acid content within the composition thus leads to improved survival of neighboring cells and improved host tissue response.

In some embodiments, the composition is prepared initially from dry components (e.g., the multivalent metal salt, the multidentate acidic organic compound, and the carbonate salt. In some embodiments, said dry components composition are present in the form of a powder or granule. As discussed herein, the adhesive composition is generally prepared by the addition of an aqueous medium to a dry mixture including a multivalent metal salt, an multidentate acidic organic compound, and a carbonate salt, e.g., calcium carbonate. In some embodiments the aqueous medium may further comprise dissolved alkali ions, e.g., sodium hydroxide. In some embodiments, the carbonate salt, e.g., calcium carbonate, sodium carbonate, etc., is added to the dry mixture of the multivalent metal salt and the multidentate acidic organic compound.

Carbonate salts are alkaline, and when used as such, the carbonate salt can act as an acidity reducing agent as discussed herein. Further, carbonate salts and acids are porogen agents, i.e., used to increase the porosity of an adhesive composition, by the release of carbon dioxide gas when dissolved in water, the release of the carbon dioxide gas from the adhesive composition during the curing process creates a plurality of pores that remain once the adhesive composition is cured (see Example 10). Without wishing to be bound by any particular theory, it is believed that smaller particles of the carbonate salt, increase the rate of carbon dioxide release upon dissolution in an aqueous medium, and this rate modulates the size of those pores that form in the cured adhesive composition. In some embodiments, the carbonate salt includes a carbonate salt or supplied as nanoparticles (e.g., diameters of about 10 nm to about 1000 nm) to microparticles (e.g., diameters of about 10 m to about 1000 m). In some embodiments, the carbonate salt includes a carbonate salt supplied as nanoparticles, e.g., particles having an average diameter of 20 nm to 200 nm. In some embodiments, the carbonate salt includes a carbonate salt supplied as nanoparticles, e.g., particles having an average diameter of 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, or 1000 nm.

In some embodiments, the adhesive composition can be mixed using devices suitable for the preparation or application of the adhesive composition, such as mixing bowls or surfaces, stirring sticks, spatulas, syringes, agitators, pre-dosed capsules, triturators, applicator hand pieces, pumps, or other preparation or delivery devices.

### Uses of the Adhesive Compositions

The adhesive compositions disclosed herein may be useful in a wide variety of applications. Exemplary uses include generation or regeneration of bone tissue, wherein the generation or regeneration of bone is derived from the increased action of osteoblast cells, wherein, the primary action of osteoblasts is to generate new mineralized bone tissue through the combination of synthetic activity, e.g., synthesis of enzymes, structural and non-structural proteins, etc., establishment and confinement of a zone of bone deposition, e.g., form a polarized matt of osteoblasts, pumping of ions, calcium, into the zone while pumping other ions out, secretion of enzymes, e.g., alkaline phosphatase, necessary for extracellular bone precursor synthesis, and concentration mineralization action of osteoblasts. Other exemplary uses include increasing the rate of bone healing or repair, inducing the formation of osteoblasts, and inducing the differentiation of bone marrow stromal cells into osteoblast cell line. Bone marrow stromal cells (“BMSCs”) are multipotential, heterogenous members within the bone marrow that act as stem/progenitor cells of the bone tissue and are indirectly responsible for hematopoicsis. BMSCs can be induced to differentiate into osteoblasts. Osteoblasts secrete alkaline phosphatase, osteoid and mineralize the bone matrix. The mineralized extracellular matrix is mainly composed of inorganic minerals, e.g., bone apatite, but also significant amounts of type I collagen, and smaller amounts of other proteins, minerals and growth factors. The directed differentiation of BMSCs can be carried out in vitro using appropriate differentiation media and can be assayed for specific markers such as presence of alkaline phosphatase (“AP”), Bone Morphogenetic protein-2 (BMP-2), and Vascular Endothelial Growth factor (VEGF).

A biomarker, e.g., an extra-cellular matrix protein, can be detected and used as evidence of osteoblast differentiation. The matrix maturation phase is characterized by maximal expression of AP. At the beginning of matrix mineralization, certain proteins are expressed, such as osteocalcin (“OC”), bone sialo-protein (“BSP”), and osteopontin (“OPN”). Once mineralization is completed, calcium deposition can be visualized using appropriate staining methods.

Traditionally, osteoconduction, osteoinduction, and osteogenesis have been used to describe various types of graft material behavior. Osteoconduction, as used herein, refers to the process of guiding the reparative growth of the natural bone through graft substance.

Osteoconduction occurs when the bone graft material serves as a scaffold for new bone growth that is promoted by surrounding native bone. Osteoblasts from the margin of the defect that is being grafted utilize the bone graft material as a framework upon which to spread and generate new bone. In some embodiments, the composition disclosed herein is osteoconductive (e.g., has osteoconductive properties).

Osteoinduction, as used herein, refers to the process of regenerating new bone cells and/or bone tissue. In some embodiments, osteoinduction involves the stimulation of undifferentiated cells to become active osteoblasts. In some embodiments, osteoinduction involves the stimulation of osteoprogenitor cells to differentiate into osteoblasts that then begin new bone formation. A bone graft material that is osteoconductive and osteoinductive will not only serve as a scaffold for currently existing osteoblasts but will also trigger the differentiation and proliferation of new osteoblasts, theoretically promoting faster integration of the graft. In some embodiments, the composition disclosed herein is osteoinductive (e.g., has osteoinductive properties). In some embodiments, the composition disclosed herein stimulates or accelerates osteoinduction in a sample or subject.

Osteogenesis may occur when vital osteoblasts originating from the bone graft material contribute to new bone growth. In some embodiments, the compositions disclosed herein comprise osteogenetic factors, e.g., multidentate acidic organic compound s, to regenerate new bone in a sample or subject. Phosphoserine may be metabolized in the body by hydrolyzing enzymes, such as phosphatases, through cleavage of the phosphate ester bond into serine and orthophosphate ion. Phosphatases involved in the in vivo metabolism of phosphoserine include alkaline phosphatase, acid phosphatase and the phosphoserine specific enzyme phosphoserine phosphatase. Several of the phosphatases may be present at the site of bone remodeling. Acid phosphatase is a product that is secreted by osteoclasts and alkaline phosphatase is a product that is secreted by osteoblasts.

In another aspect, the adhesive composition of the present disclosure is useful in the treatment of a disease or disorder in a subject. In some embodiments, the disease or disorder comprises a bone disease or disorder, e.g., cancer (e.g., osteosarcoma), osteoporosis, rickets, osteogenesis imperfecta, Paget's disease of the bone, hearing loss, renal osteodystrophy, a malignancy of the bone, infection of the bone, severe and handicapping malocclusion, osteonecrosis, or other genetic or developmental disease. In some embodiments, the compositions are used to regenerate bone in a defect caused by a disease or condition, such as cancer (e.g., osteosarcoma), osteoporosis, rickets, osteogenesis imperfecta, Paget's disease of the bone, hearing loss, renal osteodystrophy, a malignancy of the bone, infection of the bone, or other genetic or developmental disease. In some embodiments, a composition comprising an multidentate acidic organic compound is used to stimulate or accelerate bone growth in a subject that has been weakened by a disease or condition, such as cancer (e.g., osteosarcoma), osteoporosis, rickets, osteogenesis imperfecta, Paget's disease of the bone, hearing loss, renal osteodystrophy, a malignancy of the bone, infection of the bone, or other genetic or developmental disease. In some embodiments, the subject has experienced a trauma, such as a broken bone, fractured bone, or damaged tooth relating to a disease or condition, such as cancer (e.g., osteosarcoma), osteoporosis, rickets, osteogenesis imperfecta, Paget's disease of the bone, hearing loss, renal osteodystrophy, a malignancy of the bone, infection of the bone, or other genetic or developmental disease.

The adhesive compositions and methods may be used to treat a subject suffering from or afflicted with any disease or condition that impacts the structural integrity of the bony skeleton.

In some embodiments, the subject is a child. In some embodiments, the subject is an adult. In some embodiments, the subject is a senior (e.g., an adult over the age of about 50, about 55, about 60, about 65, about 70, about 75, about 80) or in a decline of the skeletal state. In some embodiments, the subject is a human or a non-human animal.

In some embodiments, the adhesive compositions and methods disclosed herein are utilized in low gravity, micro-gravity or sub-gravity conditions, e.g., as compared with the gravity conditions on Earth. In some embodiments, the diseases or disorders described herein may affect a subject differently in low gravity, microgravity or sub-gravity conditions, e.g., as compared with the gravity conditions on Earth.

In another aspect, the adhesive compositions described herein may slowly release an multidentate acidic organic compound into the surrounding medium. In another aspect, the compositions described herein may slowly release an additive into the surrounding medium. In some embodiments, the release of the multidentate acidic organic compound takes place over an extended period of time, e.g., seconds, minutes, hours, days, months, or years. In some embodiments, the release of the additive takes place over an extended period of time, e.g., seconds, minutes, hours, days, months, or years. In some embodiments, the adhesive composition is a material that solidifies in situ. In some embodiments, the adhesive composition is deposited as a depot for timed release of the multidentate acidic organic compound and/or an additive. In some embodiments, the ratio of components of the adhesive composition varies depending on the disease or condition of the subject. In some embodiments, the ratio of components in the adhesive composition varies in volumetric segments.

In some embodiments, the release of the multidentate acidic organic compound and/or additive relies on diffusion out of the depot deposit. In some embodiments, the release of the multidentate acidic organic compound and/or additive is mediated by the degradation or resorption of the adhesive composition depot deposit. In some embodiments, the release of the multidentate acidic organic compound and/or additive relies on modification of a device confining the multidentate acidic organic compound and/or additive.

In some embodiments, the release of the multidentate acidic organic compound and/or additive from the composition, in part, increases the local population of osteoblasts. The local osteoblast population may also be increased by the release of other ions from the compositions, including one or both of calcium ions or phosphate ions. In some embodiments, osteoblasts release an increase supply of alkaline phosphatase, bone morphogenetic protein (BMP 2). In some embodiments, alkaline phosphatase is responsible for metabolism and degradation of the multidentate acidic organic compound (e.g., phosphoserine) and/or additive from the adhesive composition. In some embodiments, this series of events repeats in an autocatalytic breakdown of the adhesive composition, which could accelerate the rate of subsequent bone formation by the local supply of osteoblasts that produce osteoid. In some embodiments, the release of the multidentate acidic organic compound and/or additive from the adhesive composition increases the rate and/or extent of local deposition of bone.

In some embodiments, the rate of release of the multidentate acidic organic compound and/or additive is affected by certain environmental conditions, e.g., ambient temperature, time of day, or gravity level. In some embodiments, the rate of release of the multidentate acidic organic compound and/or additive under the gravity conditions of Earth is different than the rate of release of the multidentate acidic organic compound in a micro-gravity environment.

In another aspect, the adhesive composition is applied directly to a site (e.g., into or onto bone, or in between bones or bone fragments) of a condition requiring bone tissue repair, regeneration, or generation. In some embodiments, a condition and/or site for application of the adhesive composition comprised herein include, but are not limited to, an area of a congenital bone deficit (e.g., cleft palate or other expression of a cranio-facial anomaly), an acquired condition (e.g., osteoporosis or nephrogenic osteopathy), a traumatically induced lesion (e.g., a long bone fracture, spinal compression), a site of a pathologically induced bone lesion (e.g., site of enucleation of a cyst, granuloma, site of resection of a solid tumor, an osteonecrotic segment or dysplastic tissue), a surgical defect (e.g., site of craniotomy, odontectomy, donor site for autogenous bone graft), a site where bone growth is desired for reconstructive or cosmetic reasons (e.g., orthognathic procedures, plastic surgery for mental or malar process recontouring, spinal fusion, attachment of suture or ligature, attachment of ligament, or attachment tendon, attachment of an anchor), a site of prosthetic device attachment (e.g., hip prosthesis, dental or other endosseous implant, eposteal implant, ossicular chain reconstruction, cochlear implant, amputation stump prosthesis, calvarial plate prosthesis), a site of autogenous bone graft placement (e.g., alveolar ridge reconstruction), an area at risk of disabling consequences if a bone were to collapse because of structural weakness, and where a preventive measure is instituted (e.g., osteoporotic spine, hip, long bone, or a bone weakened by pathology such as multiple myeloma, fibrous dysplasia or another).

In some embodiments, the adhesive composition is applied in a semi-fluid form. In some embodiments, the fluid is injected directly into or onto the target site of its planned activity. In some embodiments, the fluid is applied onto another object and then placed at the target site of its planned activity.

In some embodiments, the adhesive composition may be applied as a putty. In some embodiments, the adhesive composition is applied as a solid. In some embodiments, the solid is a formed or pre-formed object. In some embodiments, the pre-formed object is shaped like a missing part of the skeleton and is intended to be implanted to replace it. In some embodiments, the formed or pre-formed object is an intramedullary insert. In some embodiments, the solid is in form of a coating on another object. In some embodiments, the other object onto which the adhesive composition is applied is intended for, designed for, or used for placement in the body as an implant. In some embodiments, the other object is a dental implant. In some embodiments, the other object is an orthopedic implant. In some embodiments, the other object is an element of a joint prosthesis.

In some embodiments, the other object is an element of a limb prosthesis.

In some embodiments, the adhesive composition is deposited confined by a device. In some embodiments, the device defines the rate of release of the multidentate acidic organic compound and/or additive. In some embodiments, the device comprises metallic material. In some embodiments, the device comprises glassy material. In some embodiments, the device comprises polymeric material. In some embodiments, the device comprises material which does not persist indefinitely in the body (e.g., in the connective tissue compartment).

In some embodiments, the device comprises material which is resorbable in the body (e.g., connective tissue compartment). In some embodiments, the device comprises material which is soluble in the body (e.g., connective tissue compartment). In some embodiments, the device comprises material which is degradable (e.g., a hydrogel, scaffold, sponge, micelle, exosome) in the body (e.g., connective tissue compartment). In some embodiments, the device comprises a removable barrier. In some embodiments, the device comprises a programmable feature that controls rate of release of the multidentate acidic organic compound and/or additive. In some embodiments, the programmable feature is programmed before, during or after implementation.

In some embodiments, the adhesive composition might be deposited into the medullary space of the bone. In some embodiments, the adhesive composition might be deposited onto the external surface of the bone. In some embodiments, the adhesive composition might be applied to fractured or cut bone. In some embodiments, the adhesive composition might be applied to bone fragments. In some embodiments, the adhesive composition might be deposited to a site distant from the skeleton.

In some embodiments, the multidentate acidic organic compound is introduced locally. In some embodiments, the multidentate acidic organic compound and additive are introduced locally. In some embodiments, the multidentate acidic organic compound is introduced (e.g., introduced systemically) as a therapeutic agent, e.g., to shift the balance in the bone metabolism toward deposition of new bone. In some embodiments, the multidentate acidic organic compound and additive are introduced (e.g., introduced systemically) as a therapeutic agent, e.g., to shift the balance in the bone metabolism toward deposition of new bone.

In some embodiments, the multidentate acidic organic compound is administered as a bolus. In some embodiments, the multidentate acidic organic compound and additive are administered as a bolus. In some embodiments, the multidentate acidic organic compound is administered at a constant rate over time. In some embodiments, the multidentate acidic organic compound and additive are administered at a constant rate over time. In some embodiments, the multidentate acidic organic compound is administered in repeated dosages. In some embodiments, the multidentate acidic organic compound and additive are administered in repeated dosages.

In another aspect, bone marrow stromal cells are exposed to the adhesive composition disclosed herein or a component thereof (e.g., the multidentate acidic organic compound or the multivalent metal salt) to regenerate bone in an in vitro setting. In some embodiments, the regenerated bone cells are introduced to the site requiring bone regeneration locally or systemically.

In another aspect, there is provided a method of inducing expression of MKI67. The method may include preparing an adhesive composition, e.g., as described herein including a multivalent metal salt, an organic phosphate compound, and a carbonate salt in an aqueous medium. The method further may include applying the adhesive composition to a site (e.g., into or onto bone, or in between bones). The method additionally may include allowing the adhesive composition to harden, cure, or be resorbed by bone, the adhesive composition, upon curing, having a porosity of 10-50%, thereby inducing expression of MKI67.

In another aspect, there is provided a method of inducing amplified expression of Bone Morphogenetic Protein-2 (BMP2). The method may include preparing an adhesive composition, e.g., as described herein including a multivalent metal salt, an multidentate acidic organic compound, and a carbonate salt in an aqueous medium. The method further may include applying the adhesive composition to a site (e.g., into or onto bone, or in between bones or bone fragments). The method additionally may include allowing the adhesive composition to harden, cure, or be resorbed by bone, the adhesive composition, upon curing, having a porosity of 10-50%, thereby inducing or elevating expression of Bone Morphogenetic protein-2 (BMP2).

In another aspect, there is provided a method of inducing expression of Vascular Endothelial Growth factor (VEGF). The method may include preparing an adhesive composition, e.g., as described herein, including a multivalent metal salt, an multidentate acidic organic compound, and a carbonate salt in an aqueous medium. The method further may include applying the adhesive composition to a site (e.g., into or onto bone, or in between bones or bone fragments). The method additionally may include allowing the adhesive composition to harden, cure, or be resorbed by bone, the adhesive composition, upon curing, having a porosity of 10-50%, thereby inducing expression of Vascular Endothelial Growth factor (VEGF).

In another aspect, different variants of the components of the adhesive compositions disclosed herein may be packaged and marketed as a kit for specific indications. In some embodiments, the kit comprises a container containing an multidentate acidic organic compound (e.g., phosphoserine). In some embodiments, the kit comprises a container containing an multidentate acidic organic compound (e.g., phosphoserine) and an additive (e.g., biologically active substance). In some embodiments, the kit comprises a container containing a multivalent metal salt (e.g., calcium phosphates or calcium oxide). In some embodiments, the kit comprises a container or plurality of containers containing a multivalent metal salt (e.g., calcium phosphates or calcium oxide) and an multidentate acidic organic compound (e.g., phosphoserine) present together or in separate containers and sealed under good packaging practices to preserve the shelf life of the individual components. In some embodiments, the kit comprises a container or plurality of containers containing a multivalent metal salt (e.g., calcium phosphates, calcium carbonate, magnesium oxide) an multidentate acidic organic compound (e.g., phosphoserine), and an additive (e.g., sodium silicate, barium sulfate, biologically active factor) present together or in separate containers and sealed under good packaging practices to preserve the shelf life of the individual components. If additives are included in said kit, they may be packaged within this container or within a separate container. The aqueous medium (e.g., solution, emulsion, colloid or suspension), if included, may be provided in a separate container, or may be mixed with an multidentate acidic organic compound and/or additive. The kit may include additional components for the preparation or application of the adhesive compositions, such as mixing bowls or surfaces, stirring sticks, spatulas, syringes, or other preparation or delivery devices.

In some embodiments, the adhesive compositions may adopt a liquid, viscous, or pliable working state after mixing with an aqueous solution or suspension prior to hardening or curing, which is present for up to about 30 minutes or less, depending on the components of said adhesive compositions and the specifics of mixing technique. In some embodiments, the adhesive compositions may adopt a pliable working state for less than or equal to about 30 minutes after mixing with an aqueous solution or suspension, e.g., less than about 20 minutes, less than about 15 minutes, less than about 10 minutes, less than about 5 minutes, less than about 3 minutes, less than about 2 minutes, less than about 1 minute, less than about 30 seconds, less than about 5 seconds after mixing with an aqueous solution or suspension.

In some embodiments, after a set amount of time, the adhesive compositions may adopt a hard, cement-like state. This process of conversion from the pliable working state to the cement-like state may be referred to as hardening or curing. In some embodiments, the adhesive compositions may exhibit an adhesive strength in the cement-like state in the range of about 100 kPa to about 12,000 kPa, depending on the application and the particular components and ratios of components in said adhesive compositions. In some embodiments, the adhesive strength of the adhesive compositions in the cement-like state is between about 100 kPa and e.g., about 10,000 kPa, about 9,000 kPa, about 8,000 kPa, about 7,000 kPa, about 6,000 kPa, about 5,000 kPa, about 4,000 kPa, about 3,000 kPa, about 2,000 kPa, about 1,000 kPa, about 750 kPa, about 500 kPa, about 250 kPa, or about 200 kPa. In some embodiments, the adhesive strength of the adhesive compositions in the cement-like state is between about 100 kPa, about 200 kPa, about 300 kPa, about 400 kPa, about 500 kPa, about 600 kPa, about 700 kPa, about 800 kPa, about 900 kPa, about 1,000 kPa, about 2,500 kPa, about 5,000 kPa, about 7,500 kPa, about 10,000 kPa or about 12,000 kPa. In some embodiments, the adhesive strength of the adhesive compositions in the cement-like state is in the range of about 200 kPa and about 2,500 kPa.

In some embodiments, the particular components of the adhesive compositions may be selected to achieve the desired strength depending on the intended use of the adhesive compositions. In all embodiments, a practitioner or end user may alter the specific components to achieve the desired adhesive properties of said adhesive composition based on the intended use or desired outcome.

In some embodiments, the sequence of adding the solid components may include simultaneous bringing in contact of all of the solid components with the aqueous medium. In some embodiments, the sequence of adding the solid components may include staged bringing in contact of the solid components with the aqueous medium, e.g., powders may be mixed with the aqueous medium first and the granules might be admixed at a later time for a particular intended use or improved outcome.

### ENUMERATED EMBODIMENTS

1. An adhesive composition comprising:
   i) a multivalent metal salt;
   ii) an multidentate acidic organic compound;
   iii) a porogen; and
   iv) an aqueous medium, wherein the adhesive composition, upon curing, has a porosity of 10-50%.
2. The adhesive composition of embodiment 1, wherein the porogen comprises a carbonate salt.
3. An adhesive composition comprising:
   i) a multivalent metal salt;
   ii) an multidentate acidic organic compound;
   iii) a carbonate salt; and
   iv) an aqueous medium,
wherein the adhesive composition, upon curing, has a porosity of 10-50%.
4. An adhesive composition comprising:
   i) a multivalent metal salt;
   ii) an multidentate acidic organic compound;
   iii) a carbonate salt; and
   iv) an aqueous medium,
wherein the adhesive composition, upon curing, comprises a plurality of pores.
5. A porous adhesive composition comprising:
   i) a multivalent metal salt;
   ii) an multidentate acidic organic compound;
   iii) a carbonate salt; and
   iv) an aqueous medium,
wherein the porous adhesive composition, upon curing, has a plurality of pores having a pore size of between 20 μm to 200 m.
6. The adhesive composition of any of embodiments 1-5, wherein the adhesive composition has a density of 0.75 to 1.40 g/cm³.
7. The adhesive composition of any of embodiments 1-5, wherein a pH of the surrounding milieu is less than 8.5.
8. The adhesive composition of any of embodiments 1-5, wherein a pH of the adhesive composition in a tacky state is between 6 to 10.
9. The adhesive composition of any of embodiments 1-5, the multivalent metal salt comprises calcium phosphate, e.g., tetracalcium phosphate or tricalcium phosphate, e.g., α-tricalcium phosphate or s-tricalcium phosphate, calcium citrate, calcium carbonate, magnesium phosphate, sodium silicate, lithium phosphate, titanium phosphate, strontium phosphate, zinc phosphate, calcium oxide, magnesium oxide, calcium silicate, or a combination thereof.
10. The adhesive composition of any one of embodiments 1-5, wherein the multidentate acidic organic compound is a compound of Formula (I) or a salt thereof:

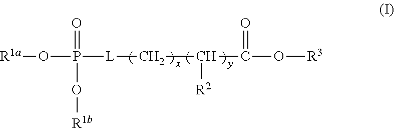

wherein:
   L is O, S, NH, or CH₂;
   each of R^{1a} and R^{1b} is independently H, optionally substituted alkyl, or optionally substituted aryl;
   R² is H, NR^{4a}R^{4b}, C(O)R⁵, or C(O)OR⁵;
   R³ is H, optionally substituted alkyl, or optionally substituted aryl;
   each of R^{4a} and R^{4b} is independently H, C(O)R⁶, or optionally substituted alkyl;
   R⁵ is H, optionally substituted alkyl, or optionally substituted aryl;
   R⁶ is optionally substituted alkyl or optionally substituted aryl; and each of x and y is independently 0, 1, 2, or 3.
11. The adhesive composition of embodiment 10, wherein the multidentate acidic organic compound comprises phosphoserine.
12. The adhesive composition of any one of embodiments 1-5, wherein the multidentate acidic organic compound is present within the adhesive composition in an amount between 10% and 90% (w/w) of the total weight.
13. The adhesive composition of any one of embodiments 1-5, wherein the aqueous medium comprises water, saliva, saline, serum, plasma, or blood.
14. The adhesive composition of any one of embodiments 1-5, wherein the adhesive composition further comprises an additive.
15. The adhesive composition of embodiment 14, wherein the additive comprises a salt, filler, formulation base, viscosity modifier, abrasive, coloring agent, flavoring agent, or polymer.
16. The adhesive composition of embodiment 15, wherein the polymer comprises poly(L-lactide), poly(D,L-lactide), polyglycolide, poly(ε-caprolactone), poly(teramethylglycolic-acid), poly(dioxanone), poly(hydroxybutyrate), poly(hydroxyvalerate), poly(lactide-co-glycolide), poly(glycolide-co-trimethylene carbonate), poly(glycolide-co-caprolactone), poly(glycolide-co-dioxanone-co-trimethylene-carbonate), poly(tetramethylglycolic-acid-co-dioxanone-co-trimethylenecarbonate), poly(glycolide-co-caprolactone-co-lactide-co-trimethylene-carbonate), poly(hydroxybutyrate-co-hydroxyvalerate), poly(methylmethacrylate), poly(acrylate), polyamines, polyamides, polyimidazoles, poly(vinyl-pyrrolidone), collagen, silk, chitosan, hyaluronic acid, gelatin, or a mixture thereof.
17. The adhesive composition of any one of embodiments 1-5, wherein the carbonate salt comprises calcium carbonate or calcium hydrogen carbonate.
18. The adhesive composition of any one of embodiments 1-5, wherein the adhesive composition during the tacky state has a tack stress of between about 10 kPa and about 250 kPa after mixing with the aqueous medium.
19. The adhesive composition of any one of embodiments 1-5, wherein the adhesive composition has a putty state for up to 15 minutes after mixing with the aqueous medium.
20. The adhesive composition of embodiment 19, wherein the adhesive composition during the putty state has a tack stress of between about 10 kPa and about 250 kPa after mixing with the aqueous medium.
21. The adhesive composition of embodiment 20, wherein the adhesive composition has an adhesive strength upon curing of greater than 100 kPa.
22. The adhesive composition of any one of embodiments 1-5, wherein the multivalent metal compound is provided as a powder.
23. The adhesive composition of embodiment 22, wherein the mean particle size of the powder is about 0.0001 to about 1.000 mm, about 0.0005 to about 0.001 mm, about 0.001 to about 0.025 mm, about 0.005 to about 0.015 mm, about 0.001 to about 0.250 mm, about 0.005 to about 0.150 mm, about 0.250 to about 0.750 mm, about 0.25 to about 0.50 mm, about 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm.
24. The adhesive composition of embodiment 3 or 4, wherein the pores range in size from 0.01 mm to 1.0 mm.
25. The adhesive composition of any one of embodiments 1-24, where in the aqueous medium further comprises a pH adjusting agent comprising sodium hydroxide.
26. A method of preparing an adhesive composition, comprising:
   (i) providing a mixture of a multivalent metal salt, multidentate acidic organic compound, and a carbonate or salt;
   (ii) contacting the mixture with an aqueous medium; and
   (iii) adding a pH adjusting agent to the mixture to bring a pH of the mixture to a value between 7 to 10, thereby preparing the adhesive composition.
27. A method of preparing an adhesive composition, comprising:
   (i) providing a mixture of a multivalent metal salt, multidentate acidic organic compound, and a carbonate or salt;
   (ii) contacting the mixture with an aqueous medium; and
   (iii) adding a pH adjusting agent to the mixture to evolve a gas from the mixture,
wherein the adhesive composition, upon curing, has a porosity of 10-50%, thereby preparing the adhesive composition.
28. A method of preparing an adhesive composition, comprising:
   (i) providing a mixture of a multivalent metal salt, multidentate acidic organic compound, and a carbonate or salt;
   (ii) contacting the mixture with an aqueous medium; and
   (iii) adding a pH adjusting agent to the mixture to evolve a gas from the mixture,
   wherein the adhesive composition, upon curing, has a plurality of pores having a pore size of between 20 μm to 200 m,
   thereby preparing the adhesive composition.
29. The method of any one of embodiments 26-28, wherein the pH adjusting agent is a basic pH adjusting agent.
30. The method of embodiment 29, wherein the basic pH adjusting agent comprises an alkali metal hydroxide, an alkali metal oxide, an alkaline earth metal hydroxide, an alkaline earth metal oxide, or a combination thereof.
31. The method of embodiment 30, wherein the basic pH adjusting agent comprises a basic pH adjusting agent selected from the group consisting of sodium hydroxide, potassium hydroxide, lithium hydroxide, calcium hydroxide, magnesium hydroxide, or a combination thereof.
30. The method of embodiment 29, wherein the basic pH adjusting agent comprises sodium hydroxide.
32. The method of any of one embodiments 26-31, wherein the adhesive composition has a density of 0.75 to 1.40 g/cm³.
33. The method of any of embodiments 26-32, wherein a pH of the surrounding milieu is less than 8.5.
34. The method of any of embodiments 26-33, wherein a pH of the adhesive composition in a tacky state is between 7 to 10.
35. The method of any of embodiments 26-34, the multivalent metal salt comprises calcium phosphate, e.g., tetracalcium phosphate or tricalcium phosphate, e.g., α-tricalcium phosphate or β-tricalcium phosphate, calcium citrate, calcium carbonate, magnesium phosphate, sodium silicate, lithium phosphate, titanium phosphate, strontium phosphate, zinc phosphate, calcium oxide, magnesium oxide, calcium silicate, or a combination thereof.
36. The method of any one of embodiments 26-35, wherein the multidentate acidic organic compound is a compound of Formula (I) or a salt thereof:

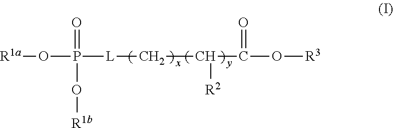

wherein:
   L is O, S, NH, or CH₂;
   each of R^{1a} and R^{1b} is independently H, optionally substituted alkyl, or optionally substituted aryl;
   R² is H, NR^{4a}R^{4b}, C(O)R⁵, or C(O)OR⁵;
   R³ is H, optionally substituted alkyl, or optionally substituted aryl;
   each of R^{4a} and R^{4b} is independently H, C(O)R⁶, or optionally substituted alkyl;
   R⁵ is H, optionally substituted alkyl, or optionally substituted aryl;
   R⁶ is optionally substituted alkyl or optionally substituted aryl; and
   each of x and y is independently 0, 1, 2, or 3.
37. The method of embodiment 36, wherein the multidentate acidic organic compound comprises phosphoserine.
38. The method of any one of embodiments 26-37, wherein the multidentate acidic organic compound is present within the adhesive composition in an amount between 10% and 90% (w/w) of the total weight.
39. The method of any one of embodiments 26-38, wherein the aqueous medium comprises water, saliva, saline, serum, plasma, or blood.
40. The method of any one of embodiments 26-39, wherein the adhesive composition further comprises an additive.
41. The method of embodiment 40, wherein the additive comprises a salt, filler, formulation base, viscosity modifier, abrasive, coloring agent, flavoring agent, or polymer.
42. The method of embodiment 41, wherein the polymer comprises poly(L-lactide), poly(D,L-lactide), polyglycolide, poly(ε-caprolactone), poly(teramethylglycolic-acid), poly(dioxanone), poly(hydroxybutyrate), poly(hydroxyvalerate), poly(lactide-co-glycolide), poly(glycolide-co-trimethylene carbonate), poly(glycolide-co-caprolactone), poly(glycolide-co-dioxanone-co-trimethylene-carbonate), poly(tetramethylglycolic-acid-co-dioxanone-co-trimethylenecarbonate), poly(glycolide-co-caprolactone-co-lactide-co-trimethylene-carbonate), poly(hydroxybutyrate-co-hydroxyvalerate), poly(methylmethacrylate), poly(acrylate), polyamines, polyamides, polyimidazoles, poly(vinyl-pyrrolidone), collagen, silk, chitosan, hyaluronic acid, gelatin, or a mixture thereof.
43. The method of any one of embodiments 26-42, wherein the carbonate or salt comprises calcium carbonate.
44. The method of any one of embodiments 26-43, wherein the adhesive composition during a tacky state has a tack stress of between about 10 kPa and about 250 kPa after mixing with the aqueous medium.
45. The method of any one of embodiments 26-44, wherein the adhesive composition has a putty state for up to 15 minutes after mixing with the aqueous medium.
46. The method of embodiment 45, wherein the adhesive composition during the putty state has a tack stress of between about 10 kPa and about 250 kPa after mixing with the aqueous medium.
47. The method of any one of embodiments 26-46, wherein the adhesive composition has an adhesive strength upon curing of greater than 100 kPa.
48. The method of any one of embodiments 26-47, wherein the multivalent metal compound is provided as a powder.
49. The method of embodiment 48, wherein a mean particle size of the powder is about 0.0001 to about 1.000 mm, about 0.0005 to about 0.001 mm, about 0.001 to about 0.025 mm, about 0.005 to about 0.015 mm, about 0.001 to about 0.250 mm, about 0.005 to about 0.150 mm, about 0.250 to about 0.750 mm, about 0.25 to about 0.50 mm, about 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm.
50. The method of embodiment 26 or 27, wherein pores range in size from 0.01 mm to 1.0 mm.
51. A method of generating or regenerating bone tissue, the method comprising:
   a) preparing an adhesive composition comprising a multivalent metal salt, an multidentate acidic organic compound, and a carbonate or salt in an aqueous medium;
   b) applying the adhesive composition to a site (e.g., into or onto bone, or in between bones or bone fragments); and
   c) allowing the adhesive composition to harden, cure, or be resorbed by bone, the adhesive composition, upon curing, having a porosity of 10-50%, thereby generating or regenerating bone tissue.
52. A method of inducing osteoblast formation, the method comprising:
   a) preparing an adhesive composition comprising a multivalent metal salt, an multidentate acidic organic compound, and a carbonate or salt in an aqueous medium;
   b) applying the adhesive composition to a site (e.g., into or onto bone, or in between bones or bone fragments); and
   c) allowing the adhesive composition to harden, cure, or be resorbed by bone, the adhesive composition, upon curing, having a porosity of 10-50%, thereby inducing osteoblast formation.
53. A method of treating or preventing a bone disease or disorder in a subject, the method comprising:
   a) preparing an adhesive composition comprising a multivalent metal salt, an multidentate acidic organic compound, and a carbonate or salt in an aqueous medium;
   b) applying the adhesive composition to a site (e.g., into or onto bone, or in between bones or bone fragments); and
   c) allowing the adhesive composition to harden, cure, or be resorbed by bone, the adhesive composition, upon curing, having a porosity of 10-50%, thereby treating or preventing the bone disease or disorder in the subject.
54. A method of inducing expression of MKI67, the method comprising:
   a) preparing an adhesive composition comprising a multivalent metal salt, an multidentate acidic organic compound, and a carbonate or salt in an aqueous medium;
   b) applying the adhesive composition to a site (e.g., into or onto bone, or in between bones or bone fragments); and
   c) allowing the adhesive composition to harden, cure, or be resorbed by bone, the adhesive composition, upon curing, having a porosity of 10-50%, thereby inducing expression of MKI67.
55. A method of inducing expression of Bone Morphogenetic protein-2 (BMP2), the method comprising:
   a) preparing an adhesive composition comprising a multivalent metal salt, an multidentate acidic organic compound, and a carbonate or salt in an aqueous medium;
   b) applying the adhesive composition to a site (e.g., into or onto bone, or in between bones or bone fragments); and
   c) allowing the adhesive composition to harden, cure, or be resorbed by bone, the adhesive composition, upon curing, having a porosity of 10-50%, thereby inducing expression of Bone Morphogenetic protein-2 (BMP2).
56. A method of inducing expression of Vascular Endothelial Growth factor (VEGF), the method comprising:
   a) preparing an adhesive composition comprising a multivalent metal salt, an multidentate acidic organic compound, and a carbonate or salt in an aqueous medium;
   b) applying the adhesive composition to a site (e.g., into or onto bone, or in between bones or bone fragments); and
   c) allowing the adhesive composition to harden, cure, or be resorbed by bone, the adhesive composition, upon curing, having a porosity of 10-50%, thereby inducing expression of Vascular Endothelial Growth factor (VEGF).
57. A method of inducing expression of one or both of caspase-3 and caspase-9, the method comprising:
   a) preparing an adhesive composition comprising a multivalent metal salt, an multidentate acidic organic compound, and a carbonate or salt in an aqueous medium;
   b) applying the adhesive composition to a site (e.g., into or onto bone, or in between bones or bone fragments); and
   c) allowing the adhesive composition to harden, cure, or be resorbed by bone, the adhesive composition, upon curing, having a porosity of 10-50%, thereby inducing expression of one or both of caspase-3 and caspase-9.
58. A method of inducing expression of Interleukin-6 (IL-6), the method comprising:
   a) preparing an adhesive composition comprising a multivalent metal salt, an multidentate acidic organic compound, and a carbonate or salt in an aqueous medium;
   b) applying the adhesive composition to a site (e.g., into or onto bone, or in between bones or bone fragments); and
   c) allowing the adhesive composition to harden, cure, or be resorbed by bone, the adhesive composition, upon curing, having a porosity of 10-50%, thereby inducing expression of Interleukin-6 (IL-6).
59. A method of inducing expression of tumor necrosis factor alpha (TNF-α), the method comprising:
   a) preparing an adhesive composition comprising a multivalent metal salt, an multidentate acidic organic compound, and a carbonate or salt in an aqueous medium;
   b) applying the adhesive composition to a site (e.g., into or onto bone, or in between bones or bone fragments); and
   c) allowing the adhesive composition to harden, cure, or be resorbed by bone, the adhesive composition, upon curing, having a porosity of 10-50%, thereby inducing expression of tumor necrosis factor alpha (TNF-α).
60. The method of any one of embodiments 51-59, wherein the adhesive composition, upon curing, comprises a plurality of pores.
61. The method of any one of embodiments 51-59, wherein the adhesive composition is porous and upon curing has a plurality of pores having a pore size of between 20 m to 200 m.
62. The method of any one of embodiments 51-61, wherein the adhesive composition further comprises a pH adjusting agent.
63. The method of embodiment 62, wherein the pH adjusting agent is a basic pH adjusting agent.
64. The method of embodiment 63, wherein the basic pH adjusting agent comprises an alkali metal hydroxide, an alkali metal oxide, an alkaline earth metal hydroxide, an alkaline earth metal oxide, or a combination thereof.
65. The method of embodiment 64, wherein the basic pH adjusting agent comprises a basic pH adjusting agent selected from the group consisting of sodium hydroxide, potassium hydroxide, lithium hydroxide, calcium hydroxide, magnesium hydroxide, or a combination thereof.
66. The method of embodiment 65, wherein the basic pH adjusting agent comprises sodium hydroxide.
67. The method of any of one embodiments 51-66, wherein the adhesive composition has a density of 0.75 to 1.40 g/cm³.
68. The method of any of embodiments 51-67, wherein a pH of the surrounding milieu is less than 8.5.
69. The method of any of embodiments 51-68, wherein a pH of the adhesive composition in a tacky state is between 6 to 10.
70. The method of any one of embodiments 51-69, the multivalent metal salt comprises calcium phosphate, e.g., tetracalcium phosphate or tricalcium phosphate, e.g., α-tricalcium phosphate or β-tricalcium phosphate, calcium citrate, calcium carbonate, magnesium phosphate, sodium silicate, lithium phosphate, titanium phosphate, strontium phosphate, zinc phosphate, calcium oxide, magnesium oxide, calcium silicate, or a combination thereof.
71. The method of any one of embodiments 51-70, wherein the multidentate acidic organic compound is a compound of Formula (I) or a salt thereof:

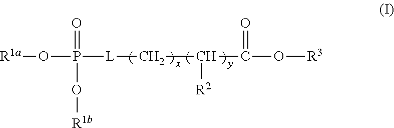

wherein:
   L is O, S, NH, or CH₂;
   each of R^{1a} and R^{1b} is independently H, optionally substituted alkyl, or optionally substituted aryl;
   R² is H, NR^{4a}R⁴, C(O)R⁵, or C(O)OR⁵;
   R³ is H, optionally substituted alkyl, or optionally substituted aryl;
   each of R^{4a} and R^{4b} is independently H, C(O)R⁶, or optionally substituted alkyl;
   R⁵ is H, optionally substituted alkyl, or optionally substituted aryl;
   R⁶ is optionally substituted alkyl or optionally substituted aryl; and
   each of x and y is independently 0, 1, 2, or 3.
72. The method of embodiment 71, wherein the multidentate acidic organic compound comprises phosphoserine.
73. The method of any one of embodiments 51-72, wherein the multidentate acidic organic compound is present within the adhesive composition in an amount between 10% and 90% (w/w) of the total weight.
74. The method of any one of embodiments 51-73, wherein the aqueous medium comprises water, saliva, saline, serum, plasma, or blood.
75. The method of any one of embodiments 51-74, wherein the adhesive composition further comprises an additive.
76. The method of embodiment 75, wherein the additive comprises a salt, filler, formulation base, viscosity modifier, abrasive, coloring agent, flavoring agent, or polymer.
77. The method of embodiment 76, wherein the polymer comprises poly(L-lactide), poly(D,L-lactide), polyglycolide, poly(ε-caprolactone), poly(teramethylglycolic-acid), poly(dioxanone), poly(hydroxybutyrate), poly(hydroxyvalerate), poly(lactide-co-glycolide), poly(glycolide-co-trimethylene carbonate), poly(glycolide-co-caprolactone), poly(glycolide-co-dioxanone-co-trimethylene-carbonate), poly(tetramethylglycolic-acid-co-dioxanone-co-trimethylenecarbonate), poly(glycolide-co-caprolactone-co-lactide-co-trimethylene-carbonate), poly(hydroxybutyrate-co-hydroxyvalerate), poly(methylmethacrylate), poly(acrylate), polyamines, polyamides, polyimidazoles, poly(vinyl-pyrrolidone), collagen, silk, chitosan, hyaluronic acid, gelatin, or a mixture thereof.
78. The method of any one of embodiments 51-77, wherein the carbonate salt comprises calcium carbonate.
79. The method of any one of embodiments 51-78, wherein the adhesive composition during a tacky state has a tack stress of between about 10 kPa and about 250 kPa after mixing with the aqueous medium.
80. The method of any one of embodiments 51-79, wherein the adhesive composition has a putty state for up to 15 minutes after mixing with the aqueous medium.
81. The method of embodiment 80, wherein the adhesive composition during the putty state has a tack stress of between about 10 kPa and about 250 kPa after mixing with the aqueous medium.
82. The method of any one of embodiments 51-81, wherein the adhesive composition has an adhesive strength upon curing of greater than 100 kPa.
83. The method of any one of embodiments 51-82 wherein the multivalent metal compound is provided as a powder.
84. The method of embodiment 83, wherein a mean particle size of the powder is about 0.0001 to about 1.000 mm, about 0.0005 to about 0.001 mm, about 0.001 to about 0.025 mm, about 0.005 to about 0.015 mm, about 0.001 to about 0.250 mm, about 0.005 to about 0.150 mm, about 0.250 to about 0.750 mm, about 0.25 to about 0.50 mm, about 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm.
85. The method of any one of embodiments 51-59, wherein pores range in size from 0.01 mm to 1.0 mm.
86. An adhesive composition comprising:
   i) a multivalent metal salt;
   ii) an multidentate acidic organic compound; and
   iii) an aqueous medium comprising sodium hydroxide.
87. An adhesive composition comprising:
   i) a multivalent metal salt;
   ii) an multidentate acidic organic compound;
   iii) a carbonate salt; and
   iv) an aqueous medium.
88. An adhesive composition comprising:
   i) a multivalent metal salt;
   ii) an multidentate acidic organic compound;
   iii) carbonic acid; and
   iv) an aqueous medium.
89. An adhesive composition comprising:
   i) a multivalent metal salt;
   ii) an multidentate acidic organic compound;
   iii) a carbonate salt; and
   iv) an aqueous medium comprising sodium hydroxide.
90. An adhesive composition comprising:
   i) a multivalent metal salt;
   ii) an multidentate acidic organic compound;
   iii) carbonic acid; and
   iv) an aqueous medium comprising sodium hydroxide.
91. The composition or method of any one of the proceeding embodiments, wherein the carbonate or salt is calcium carbonate is provided as nanoparticles having an average diameter between 20 to 200 nm.
92. The composition or method of any one of the proceeding embodiments, wherein the pH adjusting agent is sodium hydroxide that has a molar concentration between 0.5 and 5 M.
93. A method of tuning the acidity of an adhesive composition (e.g., reducing the acidity of the adhesive composition) by compounding the adhesive composition with a conjugate base of an acid (e.g., a weak acid), the method comprising:
   (i) providing a mixture of a multivalent metal salt and multidentate acidic organic compound;
   (ii) contacting the mixture with an aqueous medium; and
   (iii) adding the conjugate base of the acid (e.g., weak acid), thereby tuning the acidity of the adhesive composition (e.g., reducing the acidity of the adhesive composition). 94. The method of embodiment 93, wherein the acid is carbonic acid, citric acid, or water.
95. A method of reducing acidity and generating porosity in an adhesive composition, composition (e.g., reducing the acidity and generating porosity in the adhesive composition), the method comprising:
   (i) providing a mixture of a multivalent metal salt, multidentate acidic organic compound, and a porogen;
   (ii) contacting the mixture with an aqueous medium; and
   (iii) adding a pH adjusting agent, thereby reducing acidity and generating porosity in the adhesive composition (e.g., reducing the acidity and generating porosity in the adhesive composition).
96. The method of embodiment 95, wherein the reducing acidity and generating porosity is simultaneous or substantially simultaneous.
97. A method of controlling a pore size distribution in an adhesive composition, the method comprising:
   (i) providing a mixture of a multivalent metal salt and multidentate acidic organic compound;
   (ii) contacting the mixture with an aqueous medium; and
   (iii) adding a porogen having a controlled particle size to the mixture to effectuate a release of a gas from the adhesive composition, thereby controlling a pore size distribution in the adhesive composition.
98. The method of embodiment 97, wherein porogen comprises a nanoparticle of a carbonate salt.
99. A method of tuning porosity content in an adhesive composition without modulating acidity or ion content, the method comprising:
   (i) providing a mixture of a multivalent metal salt and multidentate acidic organic compound;
   (ii) contacting the mixture with an aqueous medium; and
   (iii) adding a varying amount of one or more calcium salts to the mixture, thereby adjusting or tuning porosity content in the adhesive composition without modulating acidity or ion content.
100. The method of embodiment 99, wherein the one or more calcium salts comprises an adjustable mixture of CaO and CaCO₃.
101. The method of embodiment 99, wherein the one or more calcium salts comprises an adjustable mixture of Ca(HCO₃)₂ and CaCO₃.
102. A method of tuning an acidity of an adhesive composition (e.g., reducing the acidity of the adhesive composition) by compounding the adhesive composition with a conjugate base of an acid (e.g., a weak acid), the method comprising:
   (i) providing a mixture of a multivalent metal salt and multidentate acidic organic compound;
   (ii) contacting the mixture with an aqueous medium; and
   (iii) adding the conjugate base of the acid (e.g., a weak acid), wherein the conjugate base does not comprise a carbonate or bicarbonate, thereby reducing or tuning the acidity of the adhesive composition (e.g., reducing the acidity of the adhesive composition).
103. The method of embodiment 102, wherein the conjugate base comprises a hydroxide salt or a tribasic citrate salt.
104. The method of any one of embodiments 93-103, wherein the pH adjusting agent is a basic pH adjusting agent.
105. The method of embodiment 104, wherein the basic pH adjusting agent comprises an alkali metal hydroxide, an alkali metal oxide, an alkaline earth metal hydroxide, an alkaline earth metal oxide, or a combination thereof.
106. The method of embodiment 105, wherein the basic pH adjusting agent comprises a basic pH adjusting agent selected from the group consisting of sodium hydroxide, potassium hydroxide, lithium hydroxide, calcium hydroxide, magnesium hydroxide, or a combination thereof.
107. The method of embodiment 104, wherein the basic pH adjusting agent comprises sodium hydroxide.
108. The method of any of one embodiments 93-107, wherein the adhesive composition has a density of 0.75 to 1.40 g/cm³.
109. The method of any of embodiments 93-108, wherein a pH of the surrounding milieu is less than 8.5.
110. The method of any of embodiments 93-109, wherein a pH of the adhesive composition in a tacky state is between 6 to 10.
111. The method of any of embodiments 93-110, the multivalent metal salt comprises calcium phosphate, e.g., tetracalcium phosphate or tricalcium phosphate, e.g., α-tricalcium phosphate or β-tricalcium phosphate, calcium citrate, calcium carbonate, magnesium phosphate, sodium silicate, lithium phosphate, titanium phosphate, strontium phosphate, zinc phosphate, calcium oxide, magnesium oxide, calcium silicate, or a combination thereof.
112. The method of any one of embodiments 93-111, wherein the multidentate acidic organic compound is a compound of Formula (I) or a salt thereof:

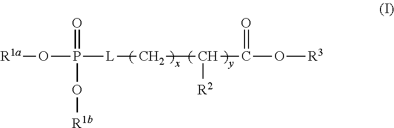

wherein:
   L is O, S, NH, or CH₂;
   each of R^{1a} and R^{1b} is independently H, optionally substituted alkyl, or optionally substituted aryl;
   R² is H, NR^{4a}R^{4b}, C(O)R⁵, or C(O)OR⁵;
   R³ is H, optionally substituted alkyl, or optionally substituted aryl;
   each of R^{4a} and R^{4b} is independently H, C(O)R⁶, or optionally substituted alkyl;
   R⁵ is H, optionally substituted alkyl, or optionally substituted aryl;
   R⁶ is optionally substituted alkyl or optionally substituted aryl; and
   each of x and y is independently 0, 1, 2, or 3.
113. The method of embodiment 112, wherein the multidentate acidic organic compound comprises phosphoserine.
114. The method of any one of embodiments 93-113, wherein the multidentate acidic organic compound is present within the adhesive composition in an amount between 10% and 90% (w/w) of the total weight.
115. The method of any one of embodiments 93-114, wherein the aqueous medium comprises water, saliva, saline, serum, plasma, or blood.
116. The method of any one of embodiments 93-115, wherein the adhesive composition further comprises an additive.
117. The method of embodiment 116, wherein the additive comprises a salt, filler, formulation base, viscosity modifier, abrasive, coloring agent, flavoring agent, or polymer.
118. The method of embodiment 117, wherein the polymer comprises poly(L-lactide), poly(D,L-lactide), polyglycolide, poly(ε-caprolactone), poly(teramethylglycolic-acid), poly(dioxanone), poly(hydroxybutyrate), poly(hydroxyvalerate), poly(lactide-co-glycolide), poly(glycolide-co-trimethylene carbonate), poly(glycolide-co-caprolactone), poly(glycolide-co-dioxanone-co-trimethylene-carbonate), poly(tetramethylglycolic-acid-co-dioxanone-co-trimethylenecarbonate), poly(glycolide-co-caprolactone-co-lactide-co-trimethylene-carbonate), poly(hydroxybutyrate-co-hydroxyvalerate), poly(methylmethacrylate), poly(acrylate), polyamines, polyamides, polyimidazoles, poly(vinyl-pyrrolidone), collagen, silk, chitosan, hyaluronic acid, gelatin, or a mixture thereof.
119. The method of any one of embodiments 93-118, wherein the carbonate or salt comprises calcium carbonate.
120. The method of any one of embodiments 93-119, wherein the adhesive composition during a tacky state has a tack stress of between about 10 kPa and about 250 kPa after mixing with the aqueous medium.
121. The method of any one of embodiments 93-120, wherein the adhesive composition has a putty state for up to 15 minutes after mixing with the aqueous medium.
122. The method of embodiment 121, wherein the adhesive composition during the putty state has a tack stress of between about 10 kPa and about 250 kPa after mixing with the aqueous medium.
123. The method of any one of embodiments 93-122, wherein the adhesive composition has an adhesive strength upon curing of greater than 100 kPa.
124. The method of any one of embodiments 93-123, wherein the multivalent metal compound is provided as a powder.
125. The method of embodiment 124, wherein a mean particle size of the powder is about 0.0001 to about 1.000 mm, about 0.0005 to about 0.001 mm, about 0.001 to about 0.025 mm, about 0.005 to about 0.015 mm, about 0.001 to about 0.250 mm, about 0.005 to about 0.150 mm, about 0.250 to about 0.750 mm, about 0.25 to about 0.50 mm, about 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm.
126. The method of any one of embodiments 95-101, wherein pores range in size from 0.01 mm to 1.0 mm.
127. The method embodiment 126, wherein the pores have a pore size of between 20 μm to 200 μm.
128. The composition or method of any one of the proceeding embodiments, wherein the carbonate salt is calcium carbonate is provided as nanoparticles having an average diameter between 20 to 200 nm.
129. The composition or method of any one of the proceeding embodiments, wherein the pH adjusting agent is sodium hydroxide that has a molar concentration between 0.5 and 5 M.
130. The composition or method of any one of the proceeding embodiments, wherein the multivalent metal salt has at least 10% of its particle size below 45 m.

### EXAMPLES

The function and advantages of these and other embodiments can be better understood from the following examples. These examples are intended to be illustrative in nature and are not considered to be in any way limiting the scope of the invention.

### Example 1. Exemplary Adhesive Compositions

Exemplary adhesive compositions are listed in Table 3. In vitro and in vivo studies described in this section evaluate several parameters of the composition, such as particle size of the porogen, particle size of the multivalent metal phosphate salts and the addition of a pH adjusting agent. These parameters were used to tune or control the physical characteristics of the compositions such as the density, porosity or pore size, the chemical characteristics of the compositions such as the pH, or the functional characteristics of the compositions such as injectability, adhesive shear strength, or compression strength, which each or in combination consequently impacts the biological response of the compositions such as the degradation or resorption rate and the resultant bone substitution rate. The composition included at least one multivalent metal phosphate salt and a multidentate acidic organic compound of Formula I as the primary components. The multivalent metal phosphate salts were calcinated to prepare different proportions of tetracalcium phosphate, α-tricalcium phosphate, and hydroxyapatite. X-Ray Diffraction (XRD) analysis was used to determine the phase composition of the multivalent metal phosphate salt as provided in Table 1. In this Example, the multivalent metal phosphate salt used in these experiments contained 80% w/w (±5%) tetracalcium phosphate, 20% w/w (±5) of α-tricalcium phosphate, and 1% w/w (±1%) hydroxyapatite.

TABLE 1
Phase Composition of multivalent metal phosphate salt

| Metal salt batch | | | | |
|---|---|---|---|---|
| 40-19061 | Sample | TTCP | α-TCP | hydroxyapatite |
| 40-19061 | 1 | 80.0 | 20.0 | — |
| >250 μm | 2 | 79.5 | 20.5 | — |
| | 3 | 77.5 | 20.5 | 1.7 |
| 40-20035 | 1 | 78.6 | 19.6 | 1.8 |
| | 2 | 78.8 | 19.4 | 1.8 |
| | 3 | 78.9 | 21.0 | 0.1 |
| 40-21086 | 1 | 78.67 | 21.1 | 0.27 |

The particle size distribution mean, d10, d50, and d90 of the multivalent metal salts were analyzed using Laser Diffraction. Particle size including are analyzed using laser diffraction. The Tornado Dry Powder System with vacuum was used where approximately 10 grams of dry powder are loaded into the module for analysis. Analysis is performed using the Fraunhofer model is used as well as a 6% obscuration. The data for different MVMS batches are shown in Table 2.

TABLE 2
Particle Size Distribution of different batches of multivalent
metal phosphate salts characterized using Laser Diffraction

| Metal Salt | | | | |
|---|---|---|---|---|
| Batch Number | d10 | d50 | d90 | Mean |
| NB019-54 | 4.247 | 19.54 | 44.95 | 22.84 |
| NB016-126 | 5.594 | 33.18 | 126.0 | 50.43 |
| NB016-87 | 145.0 | 207.9 | 280.4 | 206.6 |
| 40-19061 | 219.2 | 401.7 | 580.9 | 396.2 |
| NB020-20 | 3.91 | 21.08 | 78.59 | 33.62 |
| 40-20035 | 4.77 | 25.65 | 74.73 | 33.37 |
| 40-21048 | 4.69 | 26.47 | 110.2 | 42.38 |

### Mixing and Application

Technique 1: The dry powder components listed in Table 3, were weighed and blended in a mixing bowl. The liquid component specified was injected into the dry components to activate the powders. The components were mixed for 20-30 seconds prior to preparing each compositions. The mixed composition were back loaded into a syringe, the plunger and rod were inserted, and the composition was injected through its nozzle to deliver the composition. The compositions were used to create samples for the in vitro testing described below or applied in vivo to a bone defect site.

Technique 2: The dry and liquid components were packed in a two-compartment capsule system separated by a membrane. One compartment held the dry powder component and the second compartment held the liquid component. The capsule system contained a plunger that was pushed to introduce the liquid to the powder. The capsule was placed into a triturator and mixed for 12 s at 5000 oscillations per minute. The capsule was then placed in an applicator for immediate delivery through a cannula tip fitted at the delivery end of the capsule.

TABLE 3
List of Exemplary Compositions

| | Dry Components | | | | | | | | |
|---|---|---|---|---|---|---|---|---|---|
| | Multivalent Metal Salt | | Multidentate Organic Acid | | Non-reactive | | | | |
| | (MVMS) | | (MDOA) | | Additives | | | | |
| | | mass (g) | | mass (g) | | mass (g) | | | |
| | | (mean | | (mean | | (mean | Liquid Component | | Mixing and |
| | | particle | | particle | | particle | Aqueous | Volume | Application |
| Composition | Compound | size, μm)¹ | Compound | size, μm)¹ | Additive | size, μm)¹ | Medium | (μL) | Technique |
| Composition 1 | Multiphasic | 1.81 | O-Phospho-L-serine | 1.13 | — | — | Water | 660 | Technique 1 |
| | calcium | 22.84 | | 9.9 | | | | | |
| | phosphate | | | | | | | | |
| | (NB019-54) | | | | | | | | |
| Composition 2 | Multiphasic | 1.81 | O-Phospho-L-serine | 1.13 | — | — | 0.8M NaOH | 660 | Technique 1 |
| | calcium | 22.84 | | 9.9 | | | | | |
| | phosphate | | | | | | | | |
| | (NB019-54) | | | | | | | | |
| Composition 3 | Multiphasic | 1.81 | O-Phospho-L-serine | 1.13 | — | — | 0.8M NaOH | 675 | Technique 1 |
| | calcium | 22.84 | | 9.9 | | | | | |
| | phosphate | | | | | | | | |
| | (NB019-54) | | | | | | | | |
| | Calcium | 0.06 | | | | | | | |
| | carbonate | 0.015-0.19 | | | | | | | |
| Composition 4 | Multiphasic | 1.81 | O-Phospho-L-serine | 1.13 | — | — | 0.8M NaOH | 675 | Technique 1 |
| | calcium | 22.84 | | 9.9 | | | | | |
| | phosphate | | | | | | | | |
| | (NB019-54) | | | | | | | | |
| | Calcium | 0.03 | | | | | | | |
| | carbonate | 0.015-0.19 | | | | | | | |
| | | 0.03 | | | | | | | |
| | | 0.69-13.0 | | | | | | | |
| Composition 5 | Multiphasic | 1.81 | O-Phospho-L-serine | 1.13 | — | — | 0.8M NaOH | 675 | Technique 1 |
| | calcium | 22.84 | | 9.9 | | | | | |
| | phosphate | | | | | | | | |
| | (NB019-54) | | | | | | | | |
| | Calcium | 0.06 | | | | | | | |
| | carbonate | 0.69-13.0 | | | | | | | |
| Composition 6 | Multiphasic | 1.81 | O-Phospho-L-serine | 1.13 | — | — | 0.8M NaOII | 675 | Technique 1 |
| | calcium | 50.43 | | 9.9 | | | | | |
| | phosphate | | | | | | | | |
| | (NB019-126) | | | | | | | | |
| | Calcium | 0.06 | | | | | | | |
| | carbonate | 0.015-0.19 | | | | | | | |
| Composition 7 | Multiphasic | 1.81 | O-Phospho-L-serine | 1.13 | — | — | 0.8M NaOH | 675 | Technique 1 |
| | calcium | 206.6 | | 9.9 | | | | | |
| | phosphate | | | | | | | | |
| | (NB019-54) | | | | | | | | |
| | Calcium | 0.06 | | | | | | | |
| | carbonate | 0.015-0.19 | | | | | | | |
| Composition 8 | Multiphasic | 1.81 | O-Phospho-L-serine | 1.13 | — | — | 0.8M NaOII | 675 | Technique 1 |
| | calcium | 396.2 | | 9.9 | | | | | |
| | phosphate | | | | | | | | |
| | (40-19061) | | | | | | | | |
| | Calcium | 0.06 | | | | | | | |
| | carbonate | 0.015-0.19 | | | | | | | |
| Composition 9 | Multiphasic | 1.81 | O-Phospho-L-serine | 1.13 | — | — | Water | 660 | Technique 1 |
| | calcium | 40-19061 | | 9.9 | | | | | |
| | phosphate | 396.2 | | | | | | | |
| | (40-19061) | | | | | | | | |
| Composition 10 | Multiphasic | 1.81 | O-Phospho-L-serine | 1.13 | — | — | 0.8M NaOH | 660 | Technique 1 |
| | calcium | 396.2 | | 9.9 | | | | | |
| | phosphate | | | | | | | | |
| | (40-19061) | | | | | | | | |
| Composition 11 | Multiphasic | 1.81 | O-Phospho-L-serine | 1.13 | — | — | 0.8M NaOH | 675 | Technique 1 |
| | calcium | 396.2 | | 9.9 | | | | | |
| | phosphate | | | | | | | | |
| | (40-19061) | | | | | | | | |
| | Calcium | 0.03 | | | | | | | |
| | carbonate | 0.015-0.19 | | | | | | | |
| | | 0.03 | | | | | | | |
| | | 0.69-13.0 | | | | | | | |
| Composition 12 | Multiphasic | 1.81 | O-Phospho-L-serine | 1.13 | — | — | 0.8M NaOH | 675 | Technique 1 |
| | calcium | 396.2 | | 9.9 | | | | | |
| | phosphate | | | | | | | | |
| | (40-19061) | | | | | | | | |
| | Calcium | 0.06 | | | | | | | |
| | carbonate | 0.69-13.0 | | | | | | | |
| Composition 13a | Multiphasic | 1.63 | O-Phospho-L-serine | 1.13 | — | — | 0.8M NaOH | 675 | Technique 1 |
| | calcium | 396.2 | | 9.9 | | | | | |
| | phosphate | | | | | | | | |
| | (40-19061) | | | | | | | | |
| | Multiphasic | 0.81 | | | | | | | |
| | calcium | 22.84 | | | | | | | |
| | phosphate | | | | | | | | |
| | (NB019-54) | | | | | | | | |
| | Calcium | 0.06 | | | | | | | |
| | carbonate | 0.015-0.19 | | | | | | | |
| Composition 13b | Multiphasic | 1.27 | O-Phospho-L-serine | 1.13 | — | — | 0.8M NaOH | 675 | Technique 1 |
| | calcium | 396.2 | | 9.9 | | | | | |
| | phosphate | | | | | | | | |
| | (40-19061) | | | | | | | | |
| | Multiphasic | 0.36 | | | | | | | |
| | calcium | 22.84 | | | | | | | |
| | phosphate | | | | | | | | |
| | (NB019-54) | | | | | | | | |
| | Calcium | 0.06 | | | | | | | |
| | carbonate | 0.015-0.19 | | | | | | | |
| Composition 13c | Multiphasic | 1.45 | O-Phospho-L-serine | 1.13 | — | — | 0.8M NaOH | 675 | Technique 1 |
| | calcium | 396.2 | | 9.9 | | | | | |
| | phosphate | | | | | | | | |
| | (40-19061) | | | | | | | | |
| | Multiphasic | 0.54 | | | | | | | |
| | calcium | 22.84 | | | | | | | |
| | phosphate | | | | | | | | |
| | (NB019-54) | | | | | | | | |
| | Calcium | 0.06 | | | | | | | |
| | carbonate | 0.015-0.19 | | | | | | | |
| Composition 14 | Multiphasic | 0.369 | O-Phospho-L-serine | 0.231 | — | — | Water | 135 | Technique 2 |
| (TN-SM) | calcium | 40-20035 | | 9.9 | | | | | |
| | phosphate | | | | | | | | |
| Composition 15 | Multiphasic | 0.362 | O-Phospho-L-serine | 0.226 | — | — | 0.8M NaOH | 135 | Technique 2 |
| (TN-ISM) or | calcium | | | 9.9 | | | | | |
| (TN-SM | phosphate | | | | | | | | |
| (+NaOH + CaCO3) | (40-20035) | | | | | | | | |
| | Calcium | 0.012 | | | | | | | |
| | carbonate | 0.015-0.19 | | | | | | | |
| Composition 16 | Multiphasic | 5.6 | O-Phospho-L-serine | 0.462 | — | — | 0.8M NaOH | 1950 | Technique 1 |
| (TN-CFF) | calcium | 42.38 | | 9.9 | | | | | |
| | phosphate | | | | | | | | |
| | (40-21048) | | | | | | | | |
| Composition 17 | Multiphasic | 0.738 | O-Phospho-L-serine | 0.462 | — | — | Water | 258 | Technique 1 |
| | calcium | 33.62 | | 9.9 | | | | | |
| | phosphate | | | | | | | | |
| | (NB020-20) | | | | | | | | |
| | Magnesium | 0.0048 | | | | | | | |
| | oxide | 9.33 | | | | | | | |
| Composition 18 | Multiphasic | 0.738 | O-Phospho-L-serine | 0.462 | — | — | Water | 268 | Technique 1 |
| | calcium | 33.62 | | 9.9 | | | | | |
| | phosphate | | | | | | | | |
| | (NB020-20) | | | | | | | | |
| | Magnesium | 0.0097 | | | | | | | |
| | oxide | 9.33 | | | | | | | |
| Composition 19a | Multiphasic | 0.738 | O-Phospho-L-serine | 0.462 | — | — | Water | 280 | Technique 1 |
| | calcium | 33.62 | | 9.9 | | | | | |
| | phosphate | | | | | | | | |
| | (NB020-20) | | | | | | | | |
| | Magnesium | 0.0193 | | | | | | | |
| | oxide | 9.33 | | | | | | | |
| Composition 19b | Multiphasic | 1.476 | O-Phospho-L-serine | 0.924 | — | — | Water | 560 | Technique 1 |
| | calcium | 33.62 | | 9.9 | | | | | |
| | phosphate | | | | | | | | |
| | (NB020-20) | | | | | | | | |
| | Magnesium | 0.039 | | | | | | | |
| | oxide | 9.33 | | | | | | | |
| Composition 20 | Multiphasic | 0.738 | O-Phospho-L-serine | 0.46 | — | — | Water | 258 | Technique 1 |
| | calcium | 33.62 | | 9.9 | | | | | |
| | phosphate | | | | | | | | |
| | (NB020-20) | | | | | | | | |
| | Calcium | 0.0139 | | | | | | | |
| | silicates2 | 6.8 | | | | | | | |
| Composition 21 | Multiphasic | 0.738 | O-Phospho-L-serine | 0.46 | — | — | Water | 268 | Technique 1 |
| | calcium | 33.62 | | 9.9 | | | | | |
| | phosphate | | | | | | | | |
| | (NB020-20) | | | | | | | | |
| | Calcium | 0.0278 | | | | | | | |
| | silicates2 | 6.8 | | | | | | | |
| Composition 22 | Multiphasic | 0.738 | O-Phospho-L-serine | 0.46 | — | — | Water | 280 | Technique 1 |
| | calcium | 33.62 | | 9.9 | | | | | |
| | phosphate | | | | | | | | |
| | (NB020-20) | | | | | | | | |
| | Calcium | 0.042 | | | | | | | |
| | silicates2 | 6.8 | | | | | | | |
| Composition 23a | Multiphasic | 0.738 | O-Phospho-L-serine | 0.46 | — | — | Water | 285 | Technique 1 |
| | calcium | 33.62 | | 9.9 | | | | | |
| | phosphate | | | | | | | | |
| | (NB020-20) | | | | | | | | |
| | Calcium | 0.056 | | | | | | | |
| | silicates2 | 6.8 | | | | | | | |
| Composition 23b | Multiphasic | 1.476 | O-Phospho-L-serine | 0.924 | — | — | Water | 570 | Technique 1 |
| | calcium | 33.62 | | 9.9 | | | | | |
| | phosphate | | | | | | | | |
| | (NB020-20) | | | | | | | | |
| | Calcium | 0.056 | | | | | | | |
| | silicates2 | 6.8 | | | | | | | |
| Composition 24 | Multiphasic | 0.738 | O-Phospho-L-serine | 0.46 | — | — | Water | 258 | Technique 1 |
| | calcium | 33.62 | | 9.9 | | | | | |
| | phosphate | | | | | | | | |
| | (NB020-20) | | | | | | | | |
| | Calcium | 0.0115 | | | | | | | |
| | Citrate | 12.5 | | | | | | | |
| | tetrahydrate | | | | | | | | |
| Composition 25 | Multiphasic | 0.738 | O-Phospho-L-serine | 0.46 | — | — | Water | 268 | Technique 1 |
| | calcium | 33.62 | | 9.9 | | | | | |
| | phosphate | | | | | | | | |
| | (NB020-20) | | | | | | | | |
| | Calcium | 0.023 | | | | | | | |
| | Citrate | 6.8 | | | | | | | |
| | tetrahydrate | | | | | | | | |
| Composition 26a | Multiphasic | 0.738 | O-Phospho-L-serine | 0.46 | — | — | Water | 280 | Technique 1 |
| | calcium | 33.62 | | 9.9 | | | | | |
| | phosphate | | | | | | | | |
| | (NB020-20) | | | | | | | | |
| | Calcium | 0.046 | | | | | | | |
| | Citrate | 6.8 | | | | | | | |
| | tetrahydrate | | | | | | | | |
| Composition 26b | Multiphasic | 0.738 | O-Phospho-L-serine | 0.46 | — | — | Water | 560 | Technique 1 |
| | calcium | 33.62 | | 9.9 | | | | | |
| | phosphate | | | | | | | | |
| | (NB020-20) | | | | | | | | |
| | Calcium | 0.092 | | | | | | | |
| | Citrate | 6.8 | | | | | | | |
| | tetrahydrate | | | | | | | | |
| Composition 27 | Multiphasic | 0.738 | O-Phospho-L-serine | 0.46 | — | — | 0.1M NaOH | 258 | Technique 1 |
| | calcium | 33.62 | | 9.9 | | | | | |
| | phosphate | | | | | | | | |
| | (NB020-20) | | | | | | | | |
| Composition 28 | Multiphasic | 0.738 | O-Phospho-L-serine | 0.46 | — | | 0.4M NaOH | 258 | Technique 1 |
| | calcium | 33.62 | | 9.9 | | | | | |
| | phosphate | | | | | | | | |
| | (NB020-20) | | | | | | | | |
| Composition 29a | Multiphasic | 0.738 | O-Phospho-L-serine | 0.46 | — | — | 3.2M NaOH | 258 | Technique 1 |
| | calcium | 33.62 | | 9.9 | | | | | |
| | phosphate | | | | | | | | |
| | (NB020-20) | | | | | | | | |
| Composition 29b | Multiphasic | 1.476 | O-Phospho-L-serine | 0.924 | — | — | 3.2M NaOH | 560 | Technique 1 |
| | calcium | 33.62 | | 9.9 | | | | | |
| | phosphate | | | | | | | | |
| | (NB020-20) | | | | | | | | |
| Composition 30a | Multiphasic | 0.738 | O-Phospho-L-serine | 0.46 | — | — | Water | 258 | Technique 1 |
| | calcium | 33.62 | | 9.9 | | | | | |
| | phosphate | | | | | | | | |
| | (NB020-20) | | | | | | | | |
| Composition 30b | Multiphasic | 1.476 | O-Phospho-L-serine | 0.924 | — | — | Water | 560 | Technique 1 |
| | calcium | 33.62 | | 9.9 | | | | | |
| | phosphate | | | | | | | | |
| | (NB020-20) | | | | | | | | |
| Composition 31 | Multiphasic | 0.4 | O-Phospho-L-serine | 0.75 | — | — | Water | 135 | Technique 1 |
| | calcium | 22.84 | | 9.9 | | | | | |
| | phosphate | | | | | | | | |
| Composition 32 | Multiphasic | 8 | O-Phospho-L-serine | 5 | — | — | 2M NaOH | 3240 | Technique 1 |
| | calcium | 22.84 | | 9.9 | | | | | |
| | phosphate | | | | | | | | |
| | (NB019-54) | | | | | | | | |
| | Calcium | 2.6 | | | | | | | |
| | carbonate | 0.015-0.19 | | | | | | | |
| Composition 33 | Multiphasic | 0.6 | O-Phospho-L-serine | 0.375 | Pre-cured | 1.19 | 2M NaOH | 3240 | Technique 1 |
| | calcium | 22.84 | | 9.9 | composition | 500- | | | |
| | phosphate | | | | 32- | 700 μm | | | |
| | (NB019-54) | | | | granulated | | | | |
| | Calcium | 0.087 | | | | | | | |
| | carbonate | 0.015-0.19 | | | | | | | |
| Composition 34 | Multiphasic | 0.6 | O-Phospho-L-serine | 0.375 | Pre-cured | 1.19 | 2M NaOH | 3240 | Technique 1 |
| | calcium | 22.84 | | 9.9 | composition | 700- | | | |
| | phosphate | | | | 32- | 1000 μm | | | |
| | (NB019-54) | | | | granulated | | | | |
| | Calcium | 0.087 | | | | | | | |
| | carbonate | 0.015-0.19 | | | | | | | |
| Composition 35 | Multiphasic | | O-Phospho-L-serine | | Pre-cured | 1 | | | |
| | calcium | | | | composition | 500- | | | |
| | phosphate | | | | 32- | 700 μm | | | |
| | (NB019-54) | | | | granulated | | | | |
| | Calcium | | | | | | | | |
| | carbonate | | | | | | | | |
| Composition 36 | Multiphasic | | O-Phospho-L-serine | | Pre-cured | 1 | | | |
| | calcium | | | | composition | 700- | | | |
| | phosphate | | | | 32- | 1000 μm | | | |
| | Calcium | | | | granulated | | | | |
| | carbonate | | | | | | | | |
| Composition 37 | Multiphasic | 6 | O-Phospho-L-serine | 3.75 | Barium | 0.25 | 0.8M NaOH | 2025 | Technique 1 |
| | calcium | 73.9 | | 9.9 | sulfate | | | | |
| | phosphate | | | | | | | | |
| | (NB025-74) | | | | | | | | |
| | Calcium | 0.098 | | | | | | | |
| | carbonate | 0.015-0.19 | | | | | | | |
| Composition 38 | Multiphasic | 1.776 | O-Phospho-L-serine | 1.112 | — | — | 0.8M NaOH | 600 | Technique 1 |
| | calcium | 22.84 | | 9.9 | | | | | |
| | phosphate | | | | | | | | |
| | (NB019-54) | | | | | | | | |
| | Calcium | 0.087 | | | | | | | |
| | carbonate | 0.015-0.19 | | | | | | | |
| Composition 39 | Multiphasic | 1.2 | O-Phospho-L-serine | 0.75 | — | — | Water | 330 | Technique 1 |
| | calcium | 22.84 | | 9.9 | | | | | |
| | phosphate | | | | | | | | |
| | (NB019-54) | | | | | | | | |
| | Calcium | | Citric Acid | 0.0626 | | | | | |
| | carbonate | | | 467.3 | | | | | |
| | Calcium | 0.19 | | | | | | | |
| | citrate | 12.51 | | | | | | | |
| | tetrahydrate | | | | | | | | |
| Composition 40 | Multiphasic | 1.2 | O-Phospho-L-serine | 0.75 | — | — | Water | 330 | Technique 1 |
| | calcium | 22.84 | | 9.9 | | | | | |
| | phosphate | | | | | | | | |
| | (NB019-54) | | | | | | | | |
| | Calcium | 0.044 g | Citric Acid | 0.0626 | | | | | |
| | carbonate | 0.015-0.19 | | 467.3 | | | | | |
| | Calcium | 0.19 | | | | | | | |
| | citrate | 12.51 | | | | | | | |
| | tetrahydrate | | | | | | | | |
| Composition 41 | Multiphasic | 1.2 | O-Phospho-L-serine | 0.75 | — | — | Water | 330 | Technique 1 |
| | calcium | 206.84 | | 9.9 | | | | | |
| | phosphate | | | | | | | | |
| | (NB016-87) | | | | | | | | |
| | Calcium | 0.0198 | Citric Acid | 0.0626 | | | | | |
| | carbonate | 0.015-0.19 | | 467.3 | | | | | |
| | Calcium | 0.19 | | | | | | | |
| | citrate | 12.5 | | | | | | | |
| | tetrahydrate | | | | | | | | |
| Composition 42 | Multiphasic | 1.2 | O-Phospho-L-serine | 0.75 | — | — | Water | 330 | Technique 1 |
| | calcium | 206.6 | | 9.9 | | | | | |
| | phosphate | | | | | | | | |
| | (NB016-87) | | | | | | | | |
| | Calcium | 0.198 g | Citric Acid | 0.0626 | | | | | |
| | carbonate | 0.69-13.0 | | 467.3 | | | | | |
| | Calcium | 0.19 g | | | | | | | |
| | citrate | | | | | | | | |
| | tetrahydrate | | | | | | | | |
| Composition 43 | Multiphasic | 8 | O-Phospho-L-serine | 5 | — | — | 2M NaOH | 3240 | Technique 1 |
| | calcium | 206.6 | | 9.9 | | | | | |
| | phosphate | | | | | | | | |
| | (NB016-87) | | | | | | | | |
| | Calcium | 2.6 | | | | | | | |
| | carbonate | 0.015-0.19 | | | | | | | |
| Composition 44 | Multiphasic | 8 | O-Phospho-L-serine | 0.75 | — | — | Water | 330 | Technique 1 |
| | calcium | 206.6 | | 9.9 | | | | | |
| | phosphate | | | | | | | | |
| | (NB016-87) | | | | | | | | |
| | Calcium | 2.6 | | | | | | | |
| | carbonate | 0.69-13.0 | | | | | | | |
| Composition 45 | Multiphasic | 0.55 | O-Phospho-L-serine | 0.462 | — | — | Water | 410 | Technique 1 |
| | calcium | | | 9.9 | | | | | |
| | phosphate | | | | | | | | |
| | (alpha | | | | | | | | |
| | tricalcium | | | | | | | | |
| | phosphate) | | | | | | | | |
| | Calcium | | | | | | | | |
| | carbonate | | | | | | | | |
| | Calcium | 0.19 | | | | | | | |
| | silicate | 6.8 | | | | | | | |
| Composition 46 | Multiphasic | 0.55 | O-Phospho-L-serine | 0.462 | — | — | 0.8M NaOH | 410 | Technique 1 |
| | calcium | | | 9.9 | | | | | |
| | phosphate | | | | | | | | |
| | (alpha | | | | | | | | |
| | tricalcium | | | | | | | | |
| | phosphate) | | | | | | | | |
| | Calcium | | | | | | | | |
| | carbonate | | | | | | | | |
| | Calcium | 0.19 | | | | | | | |
| | silicate | 6.8 | | | | | | | |
| Composition 47 | Multiphasic | 0.55 | O-Phospho-L-serine | 0.462 | — | — | 0.8M NaOH | 410 | Technique 1 |
| | calcium | | | 9.9 | | | | | |
| | phosphate | | | | | | | | |
| | (alpha | | | | | | | | |
| | tricalcium | | | | | | | | |
| | phosphate) | | | | | | | | |
| | Calcium | 0.024 | | | | | | | |
| | carbonate | 0.015-0.13 | | | | | | | |
| | Calcium | 0.19 | | | | | | | |
| | silicate | 6.8 | | | | | | | |
| Composition 48 | Multiphasic | 0.55 | O-Phospho-L-serine | 0.462 | — | — | 0.8M NaOH | 410 | Technique 1 |
| | calcium | | | 9.9 | | | | | |
| | phosphate | | | | | | | | |
| | (alpha | | | | | | | | |
| | tricalcium | | | | | | | | |
| | phosphate) | | | | | | | | |
| | Calcium | 0.108 | | | | | | | |
| | carbonate | 0.015-0.13 | | | | | | | |
| | Calcium | 0.19 | | | | | | | |
| | silicate | 6.8 | | | | | | | |
| Composition 49 | Multiphasic | 5.6 | O-Phospho-L-serine | 0.462 | — | — | Water | 1950 | Technique 1 |
| (TN-CFF w water) | calcium | | | | | | | | |
| | phosphate | | | | | | | | |
| | (NB019-54) | | | | | | | | |
| | | 22.84 | | 9.9 | | | | | |

The compositions were incubated in 0.001 M PBS and characterized to evaluate their compression strength, density, porosity, adhesive shear and injection force. The elute was collected to measure its pH.

TABLE 3A
Compiled Data on the Compositions Listed in Table 3

| | Outcome Variables | | | | | | | |
|---|---|---|---|---|---|---|---|---|
| | Physical | | | | | | | |
| | | | | | Pore Surface | | | |
| | | | Pore size | Pore size | Area to | | | |
| | | | percentiles | percentiles | Total | Mechanical | | |
| | | | by count | by cumulative | Material | Adhesive | Compression | Injection |
| | Density | Porosity | (μm) | volume (μm) | Volume | Shear Stress | stress | Force |
| | (g/cc) | (%) | (d10, d50, d90) | (d10, d50, d90) | Ratio (mm⁻¹) | (MPa) | (MPa) | (N) |
| Composition | Mean ± s.d. | Mean ± s.d. | Mean ± s.d. | Mean ± s.d. | Mean ± s.d | Mean ± s.d. | Mean ± s.d. | Mean ± s.d. |
| Composition 1 | 1.88 ± 0.02 | 1.8 ± 0.9 | 19 ± 0, | 387 ± 74, | 0.73 ± .22 | 1.33 ± 0.05 | 37.70 ± 9.09 | 32.26 ± 10.1 |
| | | | 19 ± 0, | 796 ± 368, | | | | |
| | | | 39 ± 2 | 796 ± 368 | | | | |
| Composition 2 | 1.86 ± 0.03 | 1.8 ± 0.9 | 19 ± 0, | 462 ± 73, | 0.64 ± .27 | 1.72 ± 0.23 | 30.08 ± 7.75 | 8.51 ± 1.19 |
| | | | 19 ± 0, | 851 ± 208, | | | | |
| | | | 37 ± 2 | 851 ± 208 | | | | |
| Composition 3 | 1.32 ± 0.06 | 19.9 ± 3.8 | 19 ± 0, | 1113 ± 196, | 8.96 ± 2.50 | 1.61 ± 0.23 | 12.18 ± 2.15 | 5.27 ± 0.87 |
| | | | 25 ± 2, | 1113 ± 196, | | | | |
| | | | 66 ± 7 | 1113 ± 196 | | | | |
| Composition 4 | 1.52 ± 0.03 | 7.7 ± 2.8 | 19 ± 0, | 780 ± 106, | 4.05 ± .87 | 0.98 ± 0.27 | 13.57 ± 8.12 | 5.88 ± 0.58 |
| | | | 21 ± 5, | 780 ± 106, | | | | |
| | | | 55 ± 11 | 780 ± 106 | | | | |
| Composition 5 | 1.80 ± 0.03 | 4.1 ± 1.4 | 19 ± 0, | 428 ± 170, | 1.75 ± .60 | 0.49 ± 0.14 | 23.27 ± 11.29 | 9.82 ± 3.14 |
| | | | 23 ± 0, | 1066 ± 94, | | | | |
| | | | 51 ± 1 | 1066 ± 94 | | | | |
| Composition 6 | 1.25 ± 0.12 | 26.9 ± 8.1 | 19 ± 0, | 704 ± 181, | 8.76 ± 1.14 | 1.93 ± 0.62 | 7.43 ± 1.88 | 2.79 ± 0.57 |
| | | | 22 ± 3, | 2158 ± 672, | | | | |
| | | | 53 ± 13 | 2158 ± 672 | | | | |
| Composition 7 | 1.10 ± 0.06 | 47.8 ± 5.6 | 19 ± 0, | 579 ± 133, | 5.90 ± .29 | 2.65 ± 0.64 | 1.73 ± 0.36 | 30.83 ± 25.21 |
| | | | 19 ± 0, | 3097 ± 230, | | | | |
| | | | 40 ± 3 | 3097 ± 230 | | | | |
| Composition 8 | 1.52 ± 0.13 | 27.3 ± 6.6 | 19 ± 0, | 720 ± 112, | 5.84 ± .46 | 0.23 ± 0.31 | 2.67 ± 1.69 | 219.56 ± 41.56 |
| | | | 19 ± 0, | 2320 ± 562, | | | | |
| | | | 50 ± 10 | 2320 ± 562 | | | | |
| Composition 10 | 1.92 ± 0.07 | 4.5 ± 1.1 | 19 ± 0, | 385 ± 148, | 1.17 ± .18 | 2.18 ± 0.49 | 18.24 ± 3.38 | 234.33 ± 12.88 |
| | | | 19 ± 0, | 1390 ± 133, | | | | |
| | | | 39 ± 1 | 1390 ± 133 | | | | |
| Composition 12 | 1.29 ± 0.11 | 29.5 ± 2.8 | 19 ± 0, | 638 ± 156, | 6.27 ± .09 | 0.46 ± 0.11 | 0.75 ± 0.19 | 267.87 ± 19.64 |
| | | | 19 ± 0, | 2404 ± 102, | | | | |
| | | | 40 ± 1 | 2404 ± 102 | | | | |
| Composition 13a | 1.20 ± 0.11 | 49.5 ± 9.5 | 19 ± 0, | 411 ± 123, | 4.85 ± 1.18 | 0.3 ± 0.01 | 0.56 ± 0.67 | 254.40 ± 2.29 |
| | | | 20 ± 3, | 3362 ± 258, | | | | |
| | | | 45 ± 7 | 3362 ± 258 | | | | |
| Composition 13b | — | — | — | — | — | 0.68 | — | 252.70 ± 44.17 |
| Composition 13c | — | — | — | — | — | 0.35 ± 0.07 | — | 10.16 ± 0.94 |
| Composition 14 | | 3.0 | 30, | 532, | .96 | | | |
| T0 | | | 30, | 532, | | | | |
| | | | 62 | 532 | | | | |
| Composition 14 | | 7.1 | 30, | 756, | 1.79 | | | |
| 2 weeks | | | 30, | 1170, | | | | |
| | | | 60 | 1170 | | | | |
| Composition 15 | | 28.7 | 30, | 1089, | 11.55 | | | |
| T0 | | | 38, | 1236, | | | | |
| | | | 70 | 1236 | | | | |
| Composition 15 | | 37.7 | 30, | 1279, | 13.20 | | | |
| 2 weeks | | | 43, | 1677, | | | | |
| | | | 89 | 1677 | | | | |
| Composition 16 | 1.82 ± 0.03 | | | | | 1.14 ± 0.43 | 31.25 ± 7.2 | 36.76 ± 3.04 |
| Composition 16 | | 4.1 | 30, | 592, | 1.43 | | | |
| T0 | | | 38, | 652, | | | | |
| | | | 68 | 652 | | | | |
| Composition 16 | | 4.4 | 30, | 439, | 1.59 | | | |
| 2 weeks | | | 38, | 841, | | | | |
| | | | 66 | 841 | | | | |
| Composition 17 | | | | | | | | |
| Composition 18 | | | | | | | | |
| Composition 19 | | | | | | | | |
| Composition 19b | | | | | | 2.4 ± 0.4 | | |
| Composition 20 | | | | | | | | |
| Composition 21 | | | | | | | | |
| Composition 22 | | | | | | | | |
| Composition 23 | | | | | | | | |
| Composition 23b | | | | | | 3.1 ± 0.5 | | |
| Composition 24 | | | | | | | | |
| Composition 25 | | | | | | | | |
| Composition 26 | | | | | | | | |
| Composition 26b | | | | | | 2.9 ± 0.3 | | |
| Composition 27 | | | | | | | | |
| Composition 28 | | | | | | | | |
| Composition 29 | | | | | | | | |
| Composition 29b | | | | | | 3.2 ± 0.5 | | |
| Composition 30 | | | | | | | | |
| Composition 30b | — | — | — | — | — | 2.9 ± 0.6 | — | — |
| Composition 31 | | 29.8 | 30, | 243, | 19.72 | | | |
| T0 | | | 30, | 2245, | | | | |
| | | | 47 | 2245 | | | | |
| Composition 31 | | 44.7 | 30, | 204, | 23.24 | | | |
| T2 weeks | | | 30, | 2714, | | | | |
| | | | 47 | 2714 | | | | |
| Composition 32 | | 76.9 | 19, | 194, | 5.68 | | | |
| | | | 23, | 3619, | | | | |
| | | | 45 | 3619 | | | | |
| Composition 33 | | 24.9 | 30, | 1141, | 10.29 | | | |
| T0 | | | 38, | 1435, | | | | |
| | | | 77 | 1435 | | | | |
| Composition 33 | | 36.6 | 30, | 449, | 10.11 | | | |
| 2 weeks | | | 30, | 2464, | | | | |
| | | | 68 | 2464 | | | | |
| Composition 34 | | | | | | 1.49 ± 0.58 | 7.47 ± 2.30 | |
| Composition 34 | | 24.4 | 30, | 968, | 11.65 | | | |
| T0 | | | 38, | 968, | | | | |
| | | | 77 | 968 | | | | |
| Composition 34 | | 29.4 | 30, | 523, | 8.96 | | | |
| 2 weeks | | | 38, | 2065, | | | | |
| | | | 77 | 2065 | | | | |
| Composition 35 | | 58.5 | 30, | 115, | 12.67 | | | |
| T0 | | | 30, | 2883, | | | | |
| | | | 51 | 2883 | | | | |
| Composition 35 | | 58.3 | 30, | 107, | 12.92 | | | |
| 2 weeks | | | 30, | 2827, | | | | |
| | | | 51 | 2827 | | | | |
| Composition 36 | | 62.2 | 30, | 150, | 10.39 | | | |
| T0 | | | 30, | 2884, | | | | |
| | | | 54 | 2884 | | | | |
| Composition 36 | | 65.8 | 30, | 151, | 9.52 | | | |
| 2 weeks | | | 30, | 3114, | | | | |
| | | | 54 | 3114 | | | | |
| Composition 37 | 1.51 ± 0.18 | | | | | 0.7 ± 0.2 | 11.9 ± 3.8 | 158.3 ± 13.1 |
| Composition 37 | | 38.6 | 30, | 567, | 10.05 | | | |
| T0 | | | 38, | 2462, | | | | |
| | | | 64 | 2462 | | | | |
| Composition 37 | | 27.4 | 30, | 1114, | 6.12 | | | |
| 2 weeks | | | 38, | 1869, | | | | |
| | | | 75 | 1869 | | | | |
| Composition 38 | | | | | | | | |
| Composition 39 | 1.98 ± 0.03 | 4.4 ± .2 | 19 ± 0, | 289 ± 44, | 0.54 ± .06 | 3.23 ± 0.63 | 35.12 ± 12.17 | — |
| | | | 19 ± 0, | 1187 ± 24, | | | | |
| | | | 43 ± 2 | 1187 ± 24 | | | | |
| Composition 39 | | 4.1 | 30, | 368, | 1.64 | | | |
| | | | 30, | 796, | | | | |
| T0 | | | 60 | 796 | | | | |
| Composition 39 | | 2.7 | 30, | 308, | 1.44 | | | |
| 2 weeks | | | 30, | 308, | | | | |
| | | | 64 | 308 | | | | |
| Composition 40 | 1.26 ± 0.03 | 26.3 ± 4.3 | 19 ± 0, | 661 ± 53, | 9.71 ± .11 | 0.87 ± 0.24 | 5.78 ± 0.48 | — |
| | | | 23 ± 0, | 2199 ± 147, | | | | |
| | | | 61 ± 7 | 2199 ± 147 | | | | |
| Composition 41 | 0.95 ± 0.04 | 52.3 ± .3 | 19 ± 0, | 406 ± 9, | 11.03 ± .72 | 0.19 ± 0.02 | 1.25 ± 0.35 | — |
| | | | 19 ± 0, | 3184 ± 17, | | | | |
| | | | 36 ± 2 | 3184 ± 17 | | | | |
| Composition 42 | 1.96 ± 0.04 | 7.1 ± .2 | 19 ± 0, | 310 ± 9, | 1.19 ± .20 | 2.75 ± 2.01 | 13.61 ± 5.99 | — |
| | | | 19 ± 0, | 1664 ± 7, | | | | |
| | | | 54 ± 2 | 1664 ± 7 | | | | |
| Composition 43 | | 76.1 | 19, | 122, | 6.36 | — | | |
| | | | 19, | 2962, | | | | |
| | | | 36 | 2962 | | | | |
| Composition 44 | | 61.5 | 19, | 632, | 5.21 | — | | |
| | | | 23, | 3366, | | | | |
| | | | 64 | 3366 | | | | |
| Composition 45 | | | | | | | 1.47 ± 0.02 | |
| Composition 46 | | | | | | | 1.60 ± 0.03 | |
| Composition 47 | | | | | | | 1.24 ± 0.01 | |
| Composition 48 | | | | | | | 1.26 ± 0.03 | |
| Composition 49 | | | | | | | | |

### Experimental Setup

Cylindrical substrates for compression testing were prepared into PTFE molds with a 2:1 aspect ratio (6 mm×12 mm) per ASTM Standards D1663i and C873ii. Prior to the start of sample creation, 1 L of 0.001 M modified PBS was heated to 37° C. to mimic physiological conditions. The PTFE molds were submerged in the heated PBS prior to use. The mixing and activation of the samples were conducted using either technique 1 or technique 2 as described above. The composition was loaded into the molds and then packed to eliminate air bubbles formed during the loading step. The molds were then placed back in the 37° C. PBS for 2 minutes ±30 seconds to set before being removed from the mold. Samples were removed from the PBS between 15-20 minutes and sanded using 2000 grit sandpaper until smooth. The cylindrical substrates were removed from the mold. Height, diameter, and mass measurements were taken and recorded for density measurement. Each composition produced two cylindrical substrates which were then placed in a vial containing 1 mM PBS in a ratio of 1 g of the composition to 5 mL PBS. The vials were then placed in a shaking water bath set to 37° C. with 50 rpm oscillations.

### Compression Strength

An Instron Universal Testing System was used to test the compressive strength of the compositions at 44±4 hours of incubation. Each substrate was placed onto the center of a flat compression platen standing upright on its flat surface. A preload of approximately 1 N was used and a compressive force was applied at a rate of 1 mm/min until failure occurred. Failure was quantified by the applied load falling below 40% of the maximum load. The maximum compressive strength was recorded for each sample based on the maximum load endured and sample dimensions. The maximum injection force values for each sample were compared to the injection force specification of <103 N.

### Adhesive Shear

The adhesive shear strength of the various compositions was evaluated by adhering the composition to a cylinder of bovine bone. A 7/16″ plug cutter was used to cut cylindrical specimens from bovine femur cortical bone. The plugs were cut to approximately 10 mm in length. The flat surfaces were sanded using 2000 grit sandpaper. A PTFE mold with a 11.1 mm diameter×20 mm high cylindrical cavity was used to make the specimens. The mold and bone plug were incubated in PBS to a temperature of 37° C. just prior to sample preparation. The mold was then removed from the PBS solution and the flat surface of the bone plug was dried to remove excess liquid. The bone was placed in the mold such that the bottom surface of the bone was flush with the bottom surface of the mold. Each composition was mixed and applied to the mold cavity using a spatula. The same spatula was then used to compress the material into the mold cavity. The mold was then placed in the PBS solution for 15 minutes after which the test specimen was removed from the mold and incubated back into 37° C. PBS until further testing. The specimen was placed in the shear fixture such that the interface between the compositions and bone was centered in the gap. The specimen was then sheared at a rate of 1 mm/min until failure and the maximum force was recorded. The shear stress was calculated by dividing the maximum force (N) by the cross-sectional area at the interface of bone-composition (95.03 mm²). The maximum adhesive shear values for each composition were compared with the adhesive shear specification of >0.25 MPa.

### Injectability

The Instron injectability fixture and the upper compression platen were connected to the Instron and a 3D printed custom fixture was used to hold the syringe in place during testing. 3 cc slip tip syringes were used with an inner diameter of 2.45 mm. Each composition was mixed, loaded into the delivery syringe, and primed until the plunger was at 1.5 cc. The syringe was then placed in the testing fixture and the load was applied to the plunger to measure the force needed to inject the material from 1 minute to 2 minutes post product activation. The maximum injection force values for each sample were compared to the injection force specification of less than or equal to 103 N to determine its injectability.

### pH Measurement of the Elute

The media changes conducted at the time of the compression testing was stored for pH measurement. The media was allowed to until it equilibrated at room temperature. A HALO® wireless pH was used to attain pH measurements of all elute solutions.

Exemplary adhesive compositions are defined in Table 3. The dry powdered ingredients are supplied in a kit and mixed with an aqueous medium prior to use. In Table 1, the multivalent metal salt included mixed calcium phosphates, including tetracalcium phosphate (TTCP), α-tricalcium phosphate (α-TCP), and hydroxyapatite (HA); OPLS is O-phospho-L-serine. TOM250 was a formulation with 250 mg of OPLS for every 400 mg of multivalent metal salt. The multivalent metal salt used was not phase pure, having TTCP was the primary phase comprising at least 75% where it included a secondary phase having 15-30% α-TCP, and a smaller amount of HA. CaCO₃ was provided as nanoparticles having an average diameter between 20 to 200 nm. BaSO₄ was used as contrast agent for radiographic viewing. To maximize the cellular biocompatibility response, the aqueous medium was pH adjusted by the addition of sodium hydroxide (NaOH) at different molar concentrations as specified to increase the pH of the mixture. This acidity adjustment affected the tackiness of the adhesive and the setting/curing kinetics. The tacky adhesive compositions were allowed to cure, and within several minutes formed a solid structural matrix that bonded to bone.

The granules as listed in Composition 32 of Table 3 were produced using the adhesive composition that includes calcium phosphate, phosphoserine using the ratio of TOM 250 defined above, calcium carbonate at 16.67% w/w of the dry components, and an aqueous medium. The aqueous medium included a sodium hydroxide at a concentration of 2M to adjust the pH. After combining the aqueous medium and mixing the adhesive composition, a cylindrical disc was produced which cured for at least 30 minutes. The disc was then crushed to produce granules. The granules selected were sieved at two size ranges: n=500-710 μm, and 1=710-1000 μm.

### Example 2. Effect of Particle Size of Calcium Carbonate on Composition

Compositions 1-5 from Example 1 were prepared with varying particle size of calcium carbonate as shown in

Table 3, to investigate its effects on porosity, pore size distribution, and pH. Different particle sizes of calcium carbonate influence the rate of reaction between calcium carbonate and the aqueous media resulting in release carbon dioxide to generate pores as the composition cures over time. This difference in the porosity and the pore distribution within the composition will alter the physicochemical and biological characteristics of the composition. The data showed that the calcium carbonate nanoparticles contributed to the observed higher porosity within the formulation. The data also confirmed that the inclusion of calcium carbonate nanoparticles did not compromise the functional characteristics of the composition.

### Method

The compositions were mixed for 30 seconds and formed into a 6 mm diameter and 12 mm high cylinder and allowed to cure for 15-20 minutes. The substrates were then punched out and incubated in 0.001 M PBS in a ratio of 1 g/5 mL liquid. After curing, substrates were tested for porosity and compression and the elute was tested for pH, as shown in Table 4. The compositions were also assessed for the adhesive strength and injection force.

TABLE 3
Summary of the test plan, sample size and time points

| Test Description | Sample Size | Timepoints |
|---|---|---|
| Elution - pH | 3/Timepoint | T = 30 min, 1 hr, 2 hr, |
| | | 6 hr, 24 hr, 48 hr |
| Density and Porosity | 3/Timepoint | T = 40-48 hr |
| Compression | 3/ Timepoint | T = 40-48 hr |
| Injection Strength | 3/Timepoint | T = 0-3 mins |
| | | (at the time of curing) |
| Adhesive Shear | 3/Timepoint | T = 40-48 hours |

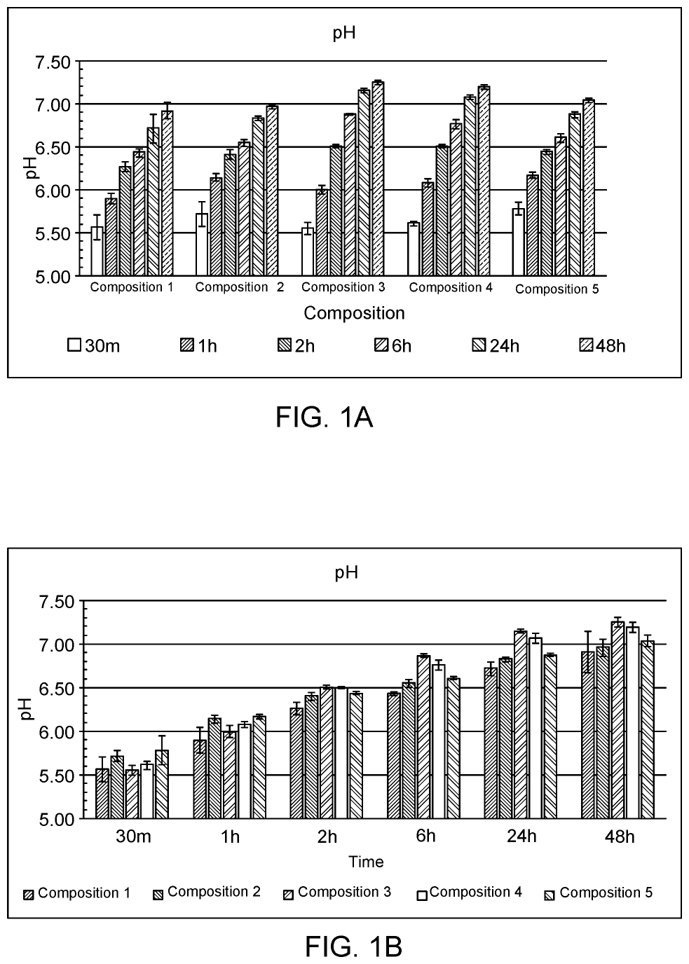

The method for each test is described in Example 1. The results are shown in the Table 5 and FIGS. 1A-1B.

TABLE 4
Results showing Density, Porosity and Compression

| | | | | | Pore Surface | | | |
|---|---|---|---|---|---|---|---|---|
| | | | Pore size | Pore size | Area to | | | |
| | | | percentiles | percentiles | Total | | | |
| | | | by count | by cumulative | Material | | | |
| | | Porosity | (μm) | volume (μm) | Volume | Compressive | Adhesive | Injection |
| | Density | (%) | (d10, d50, d90) | (d10, d50, d90) | Ratio (mm⁻¹) | Stress | Shear | Force |
| Formulation | (g/cc) | Mean ± s.d. | Mean ± s.d. | Mean ± s.d. | Mean ± s.d | (MPa) | (MPa) | (N) |
| Composition 1 | 1.876 | 1.8 ± .9 | 19 ± 0, | 387 ± 74, | .73 ± .22 | 37.70 | 1.33 | 32.26 |
| | | | 19 ± 0, | 796 ± 368, | | | | |
| | | | 39 ± 2 | 796 ± 368 | | | | |
| Composition 2 | 1.855 | 1.8 ± .9 | 19 ± 0, | 462 ± 73, | .64 ± .27 | 30.08 | 1.72 | 8.51 |
| | | | 19 ± 0, | 851 ± 208, | | | | |
| | | | 37 ± 2 | 851 ± 208 | | | | |
| Composition 3 | 1.316 | 19.9 ± 3.8 | 19 ± 0, | 1113 ± 196, | 8.96 ± 2.50 | 12.18 | 1.61 | 5.27 |
| | | | 25 ± 2, | 1113 ± 196, | | | | |
| | | | 66 ± 7 | 1113 ± 196 | | | | |
| Composition 4 | 1.524 | 7.7 ± 2.8 | 19 ± 0, | 780 ± 106, | 4.05 ± .87 | 13.57 | 0.98 | 5.88 |
| | | | 21 ± 5, | 780 ± 106, | | | | |
| | | | 55 ± 11 | 780 ± 106 | | | | |
| Composition 5 | 1.799 | 4.1 ± 1.4 | 19 ± 0, | 428 ± 170, | 1.75 ± .60 | 23.27 | 0.49 | 9.82 |
| | | | 23 ± 0, | 1066 ± 94, | | | | |
| | | | 51 ± 1 | 1066 ± 94 | | | | |

The formulation containing calcium carbonate microparticles (Composition 5) was the densest and had the lowest porosity. These values were similar to the control (Compositions 1 and 2) indicating that the micron sized calcium carbonate has little to no effect on the physical properties of the composition. The formulation containing calcium carbonate nanoparticles resulted in greater porosity which enabled more conducive biological response such as faster bone regeneration potential. The pH of the medium surrounding the cured compositions was evaluated to ensure that they are within the physiological levels. Additionally, the formulations were tested to determine the injection force and adhesive shear at the timepoints listed in Table 4. The mechanical properties of the formulation such as the injection force and the adhesive shear were not compromised by inclusion of the calcium carbonate nanoparticles.

### Example 3A. Effect of Particle Size of Multivalent Metal on Composition

Compositions 3 and 6-8 as described above were prepared with varying particle sizes of the multivalent metal salt to investigate its effects on the porosity, pH, and functional characteristics such as injection force and adhesive shear. The particle size of the multivalent metal salt affects the setting kinetics which in turn influences the physical and the functional properties of the composition. The data confirmed that multivalent metal salt up to a particle size of 250 m resulted in desirable chemical and functional characteristics in the resultant compositions.

### Method

The compositions were mixed for 30 seconds, after which the mixture was formed into a 6 mm diameter and 12 mm high cylinder and allowed to cure for 15-20 minutes. The substrate was then punched out and incubated in 0.001 M PBS in a ratio of 1 g/5 mL liquid. After curing, the substrate the pH of the elution medium was tested, porosity and compression as shown in Table 6 below.

TABLE 5
Test Description, sample size and time points

| Test Description | Sample Size | Timepoints |
|---|---|---|
| Elution - pH | 3/Timepoint | T = 30 min, 1 hr, 2 hr, |
| | | 6 hr, 24 hr, 48 hr |
| Density and Porosity | 3/Timepoint | T = 40-48 hr |
| Compression | 3/ Timepoint | T = 40-48 hr |
| Injection Strength | 3/Timepoint | T = 0-3 mins |
| | | (at the time of curing) |
| Adhesive Shear | 3/Timepoint | T = 40-48 hours |

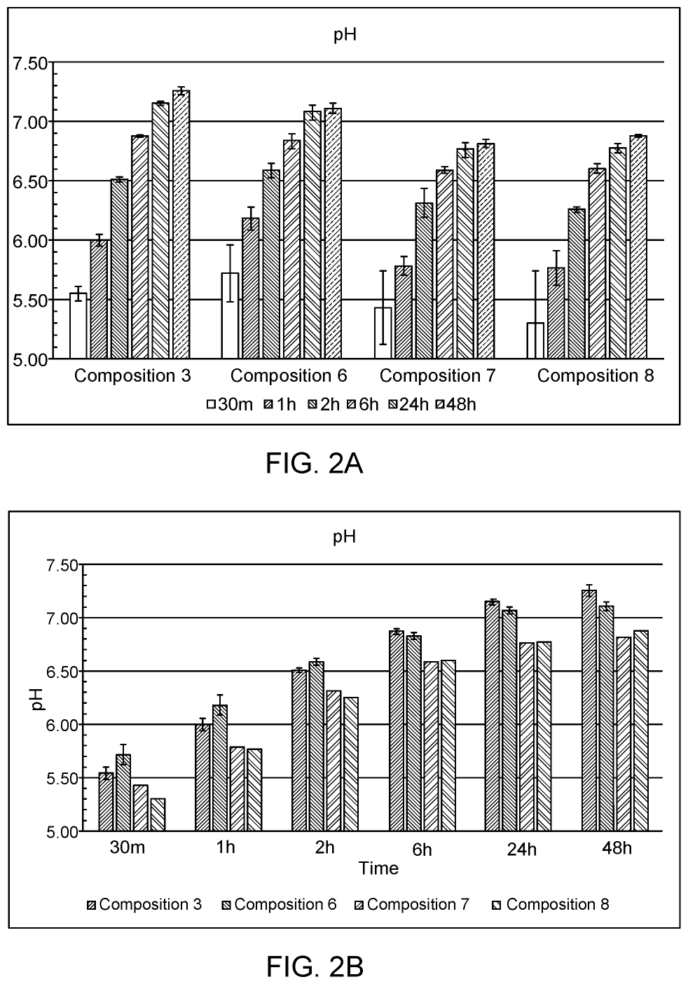

The detailed methods are described in Example 1. The compositions were tested to determine the injection force and adhesive shear. The results are presented in Table 7 and illustrated in FIGS. 2A-2B.

TABLE 6
Results of Density, Porosity and Compression

| | | | Compres- | | |
|---|---|---|---|---|---|
| | | | sive | | |
| | Density | Porosity | Stress | Adhesive | Injection |
| Formulation | (g/cc) | (%) | (MPa) | Shear | Force |
| Composition 3 | 1.32 | 27.11% | 12.18 | 1.61 | 5.27 |
| Composition 6 | 1.25 | 25.9% | 7.43 | 1.93 | 2.79 |
| Composition 7 | 1.10 | 39.6% | 1.73 | 2.65 | 30.8 |
| Composition 8 | 1.52 | | 2.67 | 0.44 | 219.6 |

The data shows that the mechanical properties of the formulation, i.e., the injection force and the adhesive shear are not compromised when the particle size of the multivalent metal salt is <250 μm.

### Example 3B. Effect of Particle Size of Multivalent Metal Phosphate Salt on Composition

The compositions with multivalent metal salts having particle sizes of >250 rm were shown to compromise functional characteristics of the resultant compositions, such as injection force and adhesive shear, as illustrated in Example 2. Here, the multivalent metal salts having particle sizes of >250 m were doped with 10%, 20% and 30% of fines, defined as multivalent metal salts with particles having diameters of <45 m. Fines were included to accelerate the reaction between multivalent metal salts and phosphoserine. The increase in the rate of reaction provided two-fold benefit: (a) faster setting of the product allowing the pores to get trapped in the matrix thereby increasing the porosity and (b) increasing the adhesive properties of the composition while making is flowable which resulted in a lower injection force.

### Method

Compositions 8, 13a, 13b, 13c were first assessed to determine adhesive shear and the injection force. The tests were conducted as per the method described in Example 1.

TABLE 7
Results of Porosity and Compression

| | Adhesive | Injection |
|---|---|---|
| Formulation | Shear | Force |
| Composition 8 | <0.2 | 219.6 |
| Composition 13a | 0.31 | 10.16 |
| Composition 13b | 0.68 | 30.57 |
| Composition 13c | 0.35 | 5.83 |

As noted, the adhesive shear and the injection force for Compositions 3 and 6-8 passed its specification with 10% fines added. This confirmed the utility of including fines at 10% by weight of the multivalent metal salts with particle size<45 μm into compositions where the multivalent metal salts primarily included particles>250 μm.

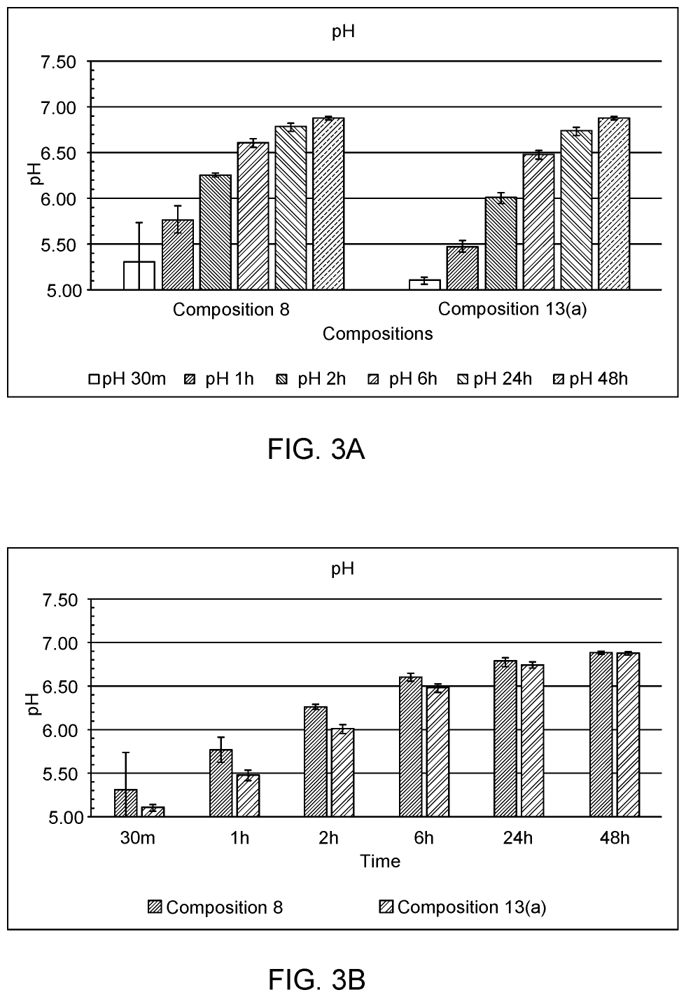

Compositions 8 and 13a were mixed for 30 seconds, after which the mixture was formed into a 6 mm diameter and 12 mm high cylinder and allowed to cure for 15-20 minutes. The substrate was then punched out and incubated in 0.001 M PBS in a ratio of 1 g/5 mL liquid. After curing, the substrate was used to measure the porosity and compression (which are compared in Table 9) and the elute were tested for pH as shown in FIGS. 3A-3B.

TABLE 9
Comparison of Functional Properties for Compositions 8 and 13a

| | | | | | Pore Surface | |
|---|---|---|---|---|---|---|
| | | | Pore size | Pore size | Area to | |
| | | | percentiles | percentiles | Total | |
| | | | by count | by cumulative | Material | |
| | | Porosity | (μm) | volume (μm) | Volume | Compressive |
| | Density | (%) | (d10, d50, d90) | (d10, d50, d90) | Ratio (mm⁻¹) | Stress |
| Composition | (g/cc) | Mean ± s.d. | Mean ± s.d. | Mean ± s.d. | Mean ± s.d | (MPa) |
| Composition 8 | 1.52 | 27.3 ± 6.6 | 27.3 ± 6.6 | 27.3 ± 6.6 | 27.3 ± 6.6 | 2.67 |
| Composition 13a | 1.19 | 19 ± 0, | 19 ± 0, | 19 ± 0, | 19 ± 0, | 0.56 |
| | | 19 ± 0, | 19 ± 0, | 19 ± 0, | 19 ± 0, | |
| | | 50 ± 10 | 50 ± 10 | 50 ± 10 | 50 ± 10 | |

The data shows that the mechanical properties of the formulation, i.e., the injection force and the adhesive shear are not compromised when the particle size of the multivalent metal salts>250 μm contains at least 10% of particles that are <45 μm.

### Example 4. In Vitro Evaluation of the Compositions Comprising CaCO₃ Nanoparticles Using Bone Marrow Stromal Cells

Several compositions were also evaluated for its biocompatibility by assaying changes in cell viability and growth rate, cell apoptosis or necrosis, and gene expression in terms of inflammation, osteoblastic differentiation, apoptosis, and collagen deposition. The controls used in this study were calcium sulfate and the negative control, i.e., treatment without the composition.

Compositions 14 (TN-SM),15 (TN-SM (+NaOH+CaCO₃), and 16 (TN-CFF), as shown in Example 1 and listed in Table 3, were mixed for 30 seconds, after which the mixture was formed into a 14 mm diameter and 1.5 mm high disc. Calcium Sulfate (CS) discs (14 mm diameter) were prepared in the mold and then plated into the well where they were left in contact with 2 mL of medium for 24 hours before starting the experiment.

### BMSCs and HGF-1 Cell Plating

After 24 hours of incubation, the tissue culture media was aspirated from each well and replaced by fresh media. The cells were then plated onto the materials and onto empty wells (negative control). Cells were plated at 10×10³ cells/well onto 96 well plates for MTT assay for Days 1, 3, 5, and 7. Cells were plated at 5×10⁴ cells/well onto 12-well plates for RNA extraction and photomicrographs for Days 1, 3, 5, and 7. The culture media was replaced on day 1 and day 4.

BMSCs were plated in α-MEM (GIBCO; catalog #12561-056) supplemented with 16.5% fetal bovine serum no-heat inactivated (Atlanta Biologicals; catalog #S11550), 2 mM L-glutamine (GIBCO; catalog #25030-081), 100 units/mL penicillin G and 100 μg/mL streptomycin sulfate (GIBCO; catalog #15140-122) at 37° C. with 5% CO₂. HGF-1 were plated in DMEM (ATCC; catalog #30-2002) supplemented with 16.5% fetal bovine serum no-heat inactivated (Atlanta Biologicals; catalog #S11550), 100 units/mL penicillin G and 100 μg/mL streptomycin sulfate (GIBCO; catalog #15140-122) at 37° C. with 5% CO₂.

### Cell Viability

Cell viability assessments demonstrated a significant improvement in cell viability between the pH-adjusted adhesive compositions (TN-CFF, TN-ISM) compared to the non-pH-adjusted adhesive composition (TN-SM). For this study, calcium sulfate (CS) was used as a positive control, while the negative control (CTRL) was just the bone marrow stromal cells (BMSCs) seeded onto the well surface without treatment. BMSCs were identified as ideal cells for the study due to their ability to differentiate into the osteoblastic lineage.

Cell viability was determined by measuring the reduction of 3-[4, 5-dimethylthiazol-2-yl]-2,5-diphenyltetrazolium bromide (MTT) (ab211091, Abeam) as measurement of the cells ability to reduce soluble-MTT (yellow) to formazan-MTT (purple). At the established times (day 1, day 3, day 5, and day 7), the media of each well was replaced by the same amount (50 μL) of serum-free media and cells were incubated with 50 L of MTT reagent for 3 hours at 37° C. Subsequently, to solubilize the reagent, the plates were incubated with 150 L of MTT solvent for 30 minutes at 37° C. After finishing the incubation with MTT solvent, an aliquot of 100 μL of the solution from each well was transferred to a fresh 96-well plate in order to record the absorbance at 590 nm. This step was performed since it is not possible to read directly through the original plates because of the presence of the materials in the wells. Spectrophotometric reading of the optical density was performed at 590 nm with a microplate reader. Values obtained in the absence of cells were considered as background.

Gene Expression Using qRT-PCR

Total RNA was isolated using PURELINK® RNA Mini Kit (Invitrogen; catalog #12183025) following the manufacturer's guidelines. cDNA was synthesized using RevertAid First Strand cDNA Synthesis (Thermo Fisher; catalog #K1621) according with the protocol. qRT-PCR was then performed loading 12.5 ng of cDNA which was amplified for 40 cycles using POWERTRACK™ SYBR™ Green Master Mix (A46109) with a STEPONEPLUS™ Real-Time PCR System. The primers used for qRT-PCR analysis are listed in Table 10.

TABLE 10
The RNA sequence used for gene expression

| Gene | Fw-sequence (5′-3′) | Rv-sequence (5′-3′) |
|---|---|---|
| BMP2 | CTACATGCTAGACCTGTATCGC | CCCACTCGTTTCTGGTAGTTC |
| RUNX2 | TTCACCTTGACCATAACCGTC | GGCGGTCAGAGAACAAACTAG |
| SP7 | CTCCCTCTCCCTTTTCTCTCT | GGAGCCATAGTGAACTTCCTC |
| ALPL | GATGTGGAGTATGAGAGTGACG | GGTCAAGGGTCAGGAGTTC |
| MKI67 | AAAAGAATTGAACCTGCGGAAG | AGTCTTATTTTGGCGTCTGGAG |
| Caspasc3 | ACTGGACTGTGGCATTGAG | GAGCCATCCTTTGAATTTCGC |
| Caspase9 | CCAACCCTAGAAAACCTTACCC | TCTGCATTTCCCCTCAAACTC |
| IL-6 | CCACTCACCTCTTCAGAACG | CATCTTTGGAAGGTTCAGGTTG |
| NOS2 | GTTTGACCAGAGGACCCAG | ATCTCCTTTGTTACCGCTTCC |
| COL1A1 | CCCCTGGAAAGAATGGAGATG | TCCAAACCACTGAAACCTCTG |
| COL1A2 | AGGACAAGAAACACGTCTGG | GGTGATGTTCTGAGAGGCATAG |
| COL3A1 | AAGTCAAGGAGAAAGTGGTCG | CTCGTTCTCCATTCTTACCAGG |
| COL4A3 | CCCACAGGCATAGAAGGATTC | GCCTATTACATGACTCCCTGG |
| COL5A1 | CGGAACCTTGACGAGAACTAC | TCTCCCTTTTGGCCTTTCTC |
| TGFB1 | GCCTTTCCTGCTTCTCATGG | TCCTTGCGGAAGTCAATGTAC |
| hPPIA | GCCGAGGAAAACCGTGTACT | CTGCAAACAGCTCAAAGGAGAC |

The expression level of a housekeeping gene (human PPIA) was determined. This gene showed low variability across all compounds tested and it was used to generate a normalization factor to which all target genes were normalized. The MTT assay was used to measure cellular metabolic activity as an indicator of cell viability, proliferation, and cytotoxicity. Cell viability was measured by means of MTT test after 1, 3, 5, and 7 days of culture. After 1 day of culture, all the compositions showed approximately the same level of viability as recorded for the negative control, i.e., cells without treatment. After 3-, 5-, and 7-days, TN-SM (+NaOH+nCaCO₃) and TN-SM showed lower level of viability with respect to the control. Cell number was higher when cells were growing on TN-SM (+NaOH+nCaCO₃) samples in comparison to TN-SM, on day 7.

### Assessment of Proliferation

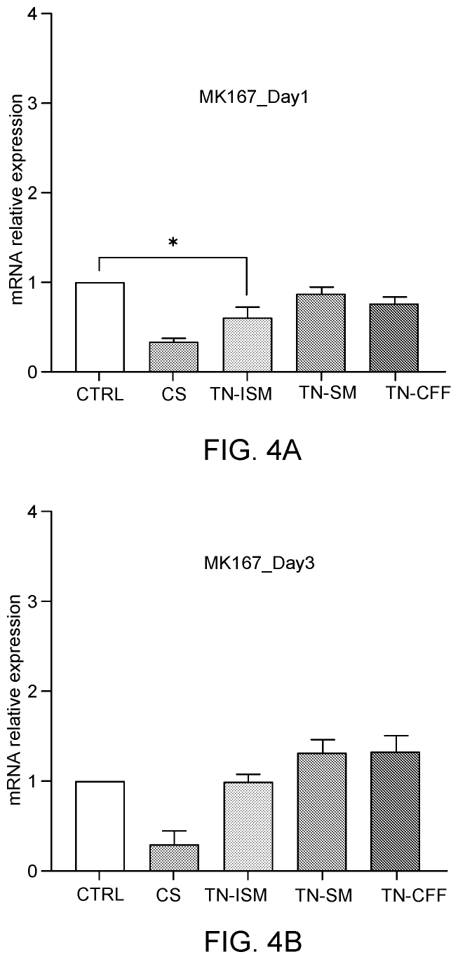

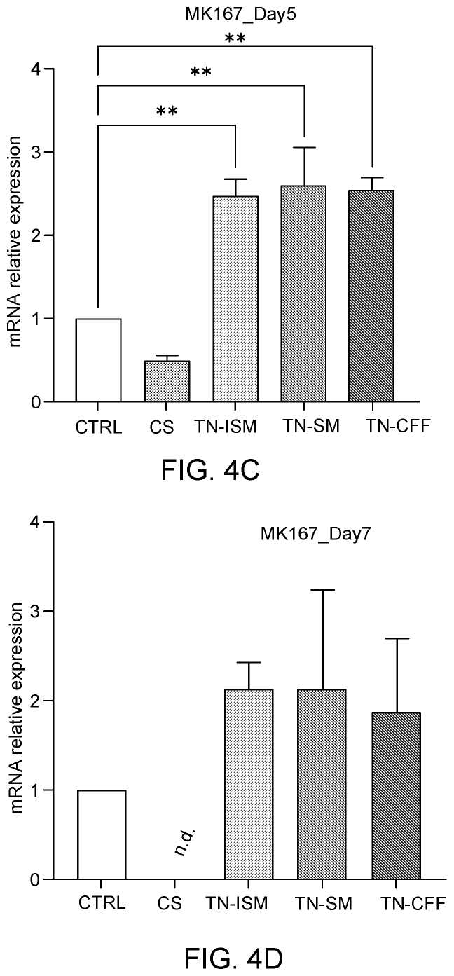

FIGS. 4A-4D showed the same level of expression of the proliferation marker KI-67 (MKI67) for all the compositions and control after 1 and 3 days. The highest level of cell viability was observed when the cells were grown in the presence of the compositions after 5 and 7 days in comparison to the control and CS. As shown in FIGS. 4A-4D, human bone marrow stromal cell viability was in general the highest with the human bone marrow stromal cells growing in the presence of the adhesive composition relative to control compositions. Following one day and three days post-deposition of the adhesive compositions, the mRNA relative expression for MKI67 was approximately the same between the adhesive compositions of this disclosure and the control composition. Specifically, the greatest level of cell viability was observed with human bone marrow stromal cells growing in the presence of the adhesive composition formulations of this disclosure after both five and seven days in comparison to the control and calcium sulfate composition. It is noted that the human bone marrow stromal cell viability was at a statistically significant maximum after five days and decreased at the seven day period. At day seven, mRNA relative expression for the calcium sulfate composition could not be determined as the human bone marrow stromal cells were not detected.

### Assessment of Differentiation

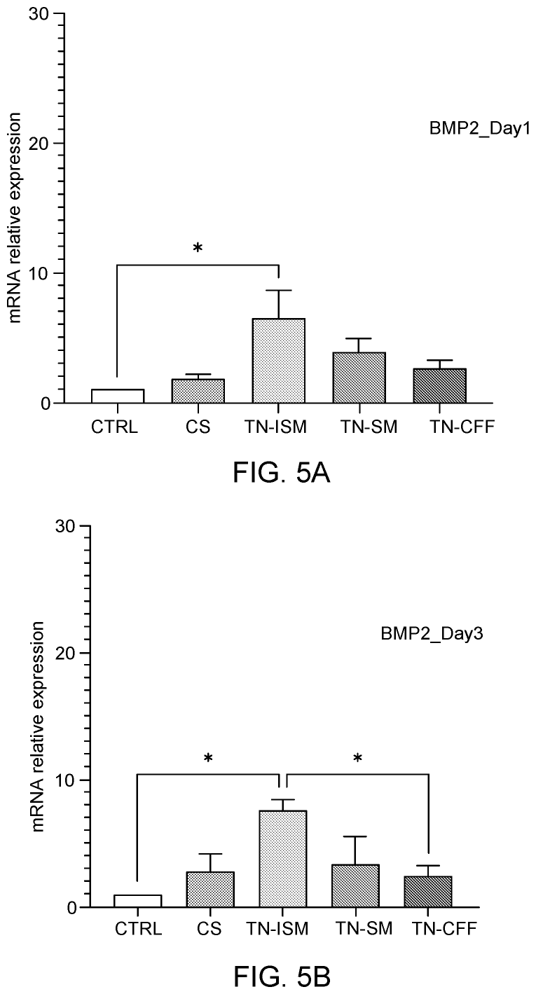

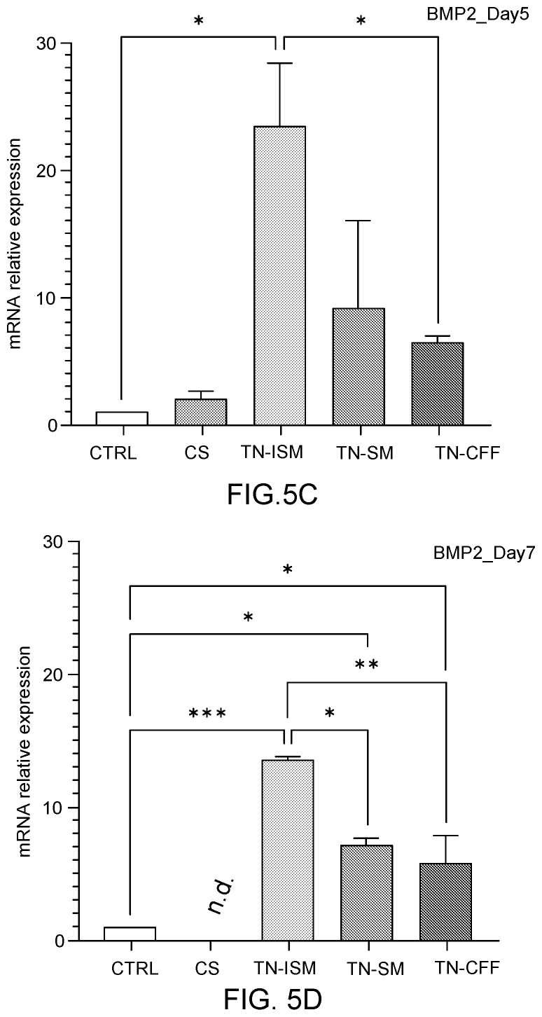

The gene expression level of BMP2, an osteogenic protein expressed in metabolically active bone and during bone regeneration, was used to evaluate BMCS differentiation. A 20-fold higher level of Bone morphogenetic protein-2 (BMP2) expression, an important early osteogenic marker, was recorded for TN-SM (+NaOH+CaCO₃) composition in comparison to the control as shown in FIGS. 5A-5D, confirming that this material does not show osteoinductive properties, but it belongs to a group of bone grafts with osteoconductive property. As shown in FIGS. 5A-5D, the mRNA relative expression of BMP2 in general was the highest with the human bone marrow stromal cells growing in the presence of the pH adjusted adhesive composition formulation (TN-ISM) independent of the time after deposition of the composition. Specifically shown in FIG. 5C, the TN-ISM formulation showed an approximately 20-fold increase in mRNA relative expression of BMP2 comparted to both the control and CS samples. The other adhesive compositions of this disclosure, TN-SM and TN-CFF, also showed increased mRNA relative expression of BMP2 relative to the CS and control compositions. In contrast, the CS composition showed low levels of mRNA relative expression of BMP2, confirming that this materials does not show osteoinductive properties. This illustrates the benefits of addition of nano-size calcium carbonate in the composition.

### Assessment of Collagen Deposition

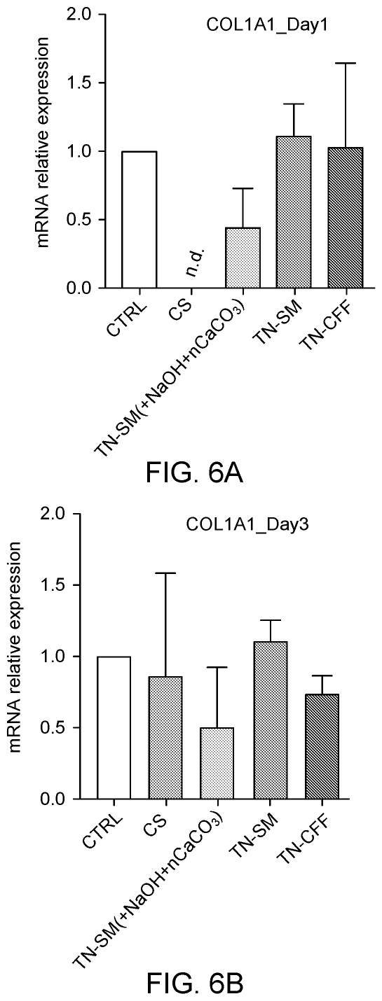

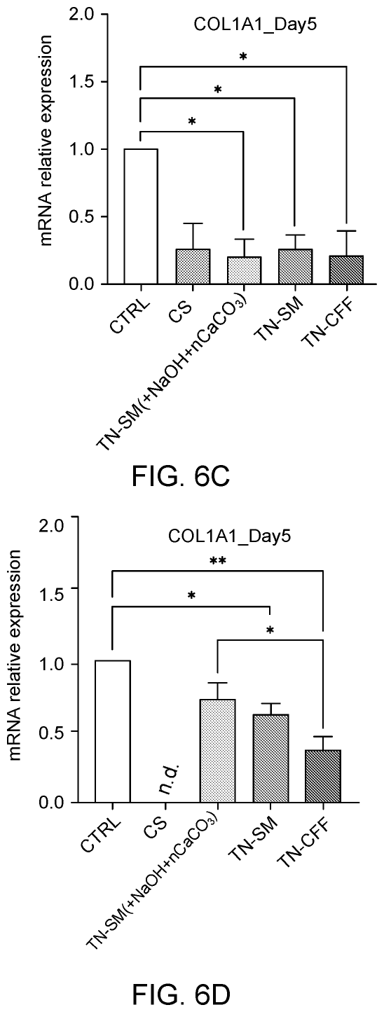

The expression of the collagen gene COL1A1, a key factor in collagen formation and deposition, showed a decrease at 3-5- and 7-days as shown in FIGS. 6A-6D. The same trend was observed for the positive control, calcium sulfate.

### Assessment of Apoptosis

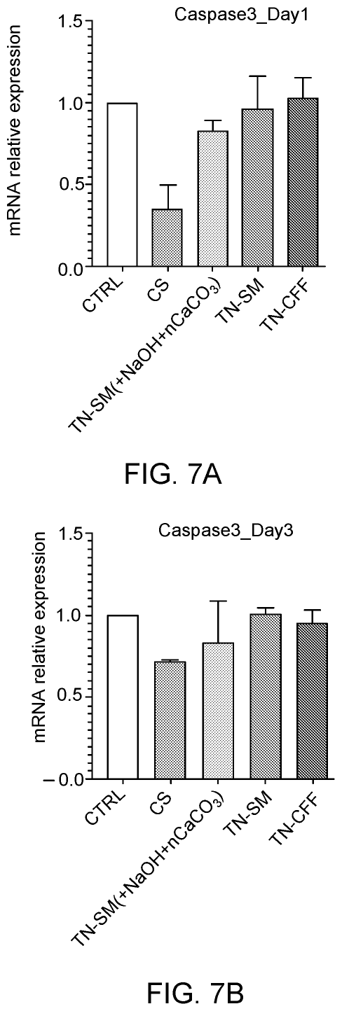

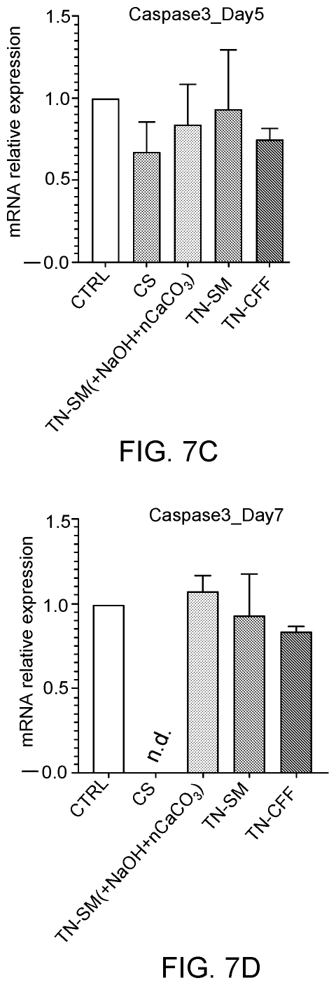

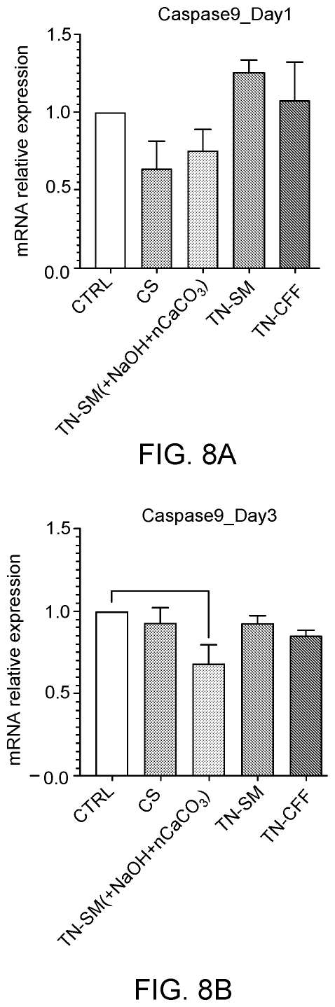

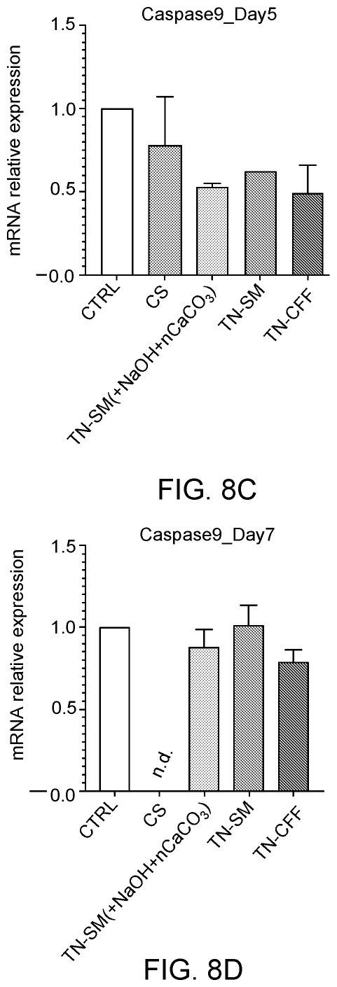

The expression of Caspase-3, a key factor in apoptosis execution and Caspase-9, an important factor in apoptosis activation showed that Caspase-3 levels were approximately the same for all the samples at each time point as shown in FIGS. 7A-7D. The expression of Caspase-9 decreased when the cells were incubated on TN-SM (+NaOH+CaCO₃) at each time point as shown in FIGS. 8A-8D. The decrease was quite significant after 3 days of culture, in comparison to the other test groups. This confirms the benefits of addition of calcium carbonate nanoparticles in the compositions.

### Assessment of Biomineralization

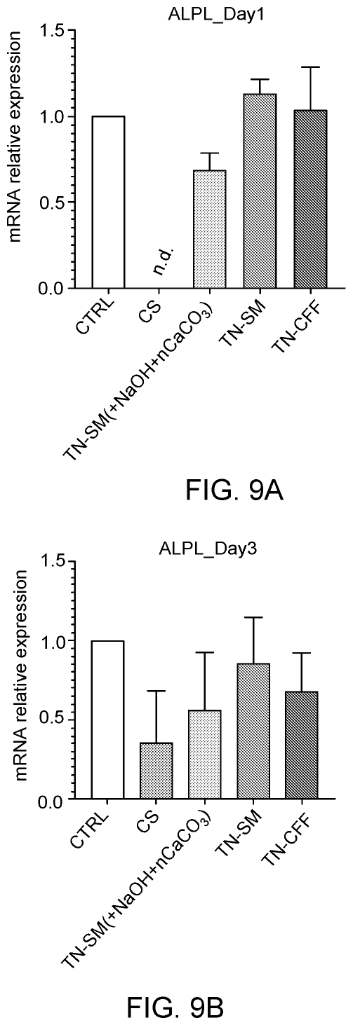

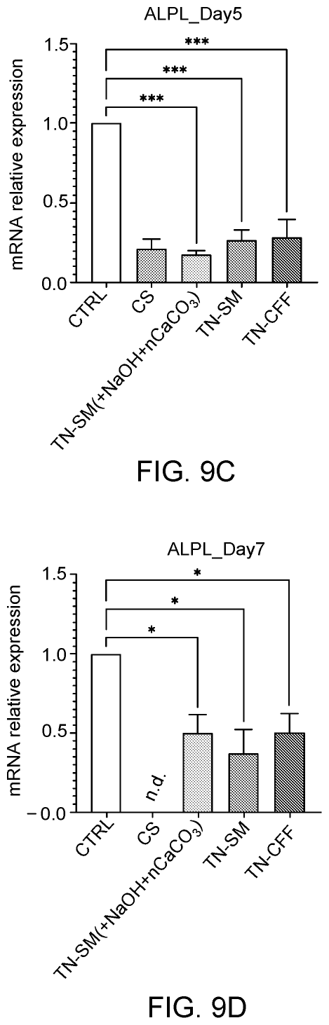

The expression of the ALPL gene, a key factor is producing an enzyme called tissue-nonspecific alkaline phosphatase (TNSALP) and playing an important role in the growth and development of bones and teeth, showed a decrease in expression at 5- and 7-days as illustrated in FIGS. 9A-9D. The same trend was observed for the positive control, calcium sulfate control at 3 days.

### Assessment of Angiogenesis

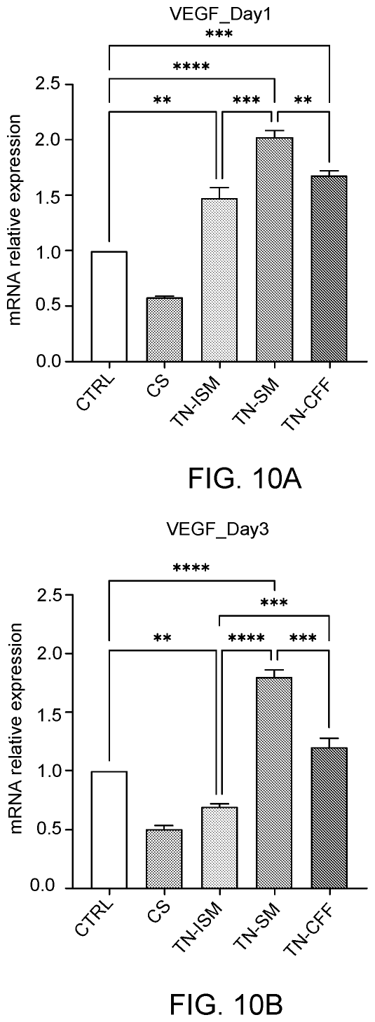

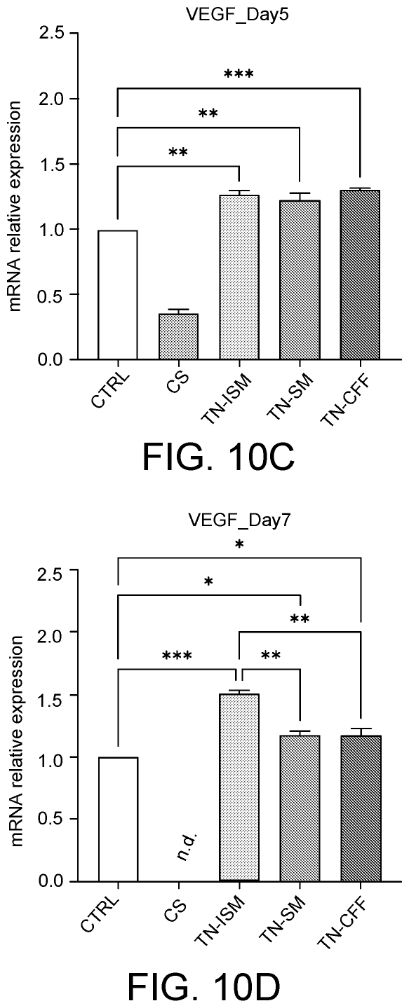

Angiogenesis was assessed by evaluating the expression of Vascular Endothelial Growth Factor (VEGF) gene as shown in FIGS. 10A-10D. The analysis showed the mRNA relative expression of VEGF in general was the highest with the human bone marrow stromal cells growing in the presence of the formulations of this disclosure relative to the control and CS compositions independent of the time after deposition of the composition.

### Example 5. In Vivo Evaluation of Compositions Comprising CaCO₃ Nanoparticles in the New Zealand White Rabbit (NZWR) Distal Femur Critical Size Defect Model

In this example, Compositions 14 and 15 as listed in Table 3 were implanted into the lateral aspect of the metaphyseal-epiphyseal distal femur of four NZWRs and evaluated at 3 and 8 weeks via Cone-beam computed tomography (CBCT) and histology.

All rabbits reached the study endpoints of 3 and 8 weeks without any complications. CBCT did not reveal any significant difference in TN-SM substitution between timepoints. TN-ISM was characterized by a decrease in radio-opacity of TN-ISM by 8-weeks, suggestive of increased bone substitution. These findings were corroborated with histology. TN-SM was characterized by minimal bone ingrowth at both timepoints. TN-ISM displayed increased eosinophilic trabecular bone and osteoid deposition that increased from 3 weeks to 8 weeks, evidenced by thickening of bone trabecula that more closely resembled native bone trabecula. The increased bone and osteoid deposition for TN-ISM was attributed to the porosity provided by nCaCO₃. Therefore, the addition of 2% nCaCO₃ resulted in increased bone substitution for TN-ISM in the rabbit distal femur model compared to TN-SM, evidenced by increased trabecular bone formation at both 3 and 8 weeks.

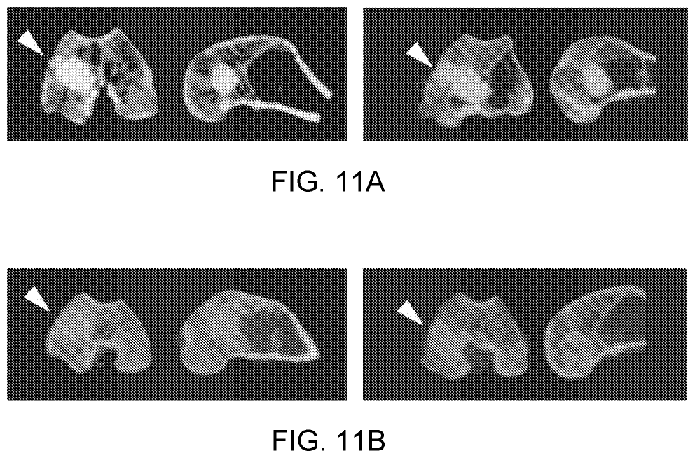

Representative CBCT reconstructions are shown in FIGS. 11A-11B. 2D image cross-sections were acquired to visualize the composition formulations and the composition-bone interface. There were no signs of the compositions loosening, fracturing, or migrating. The composition appeared to be in situ within the defect and the margins appeared to be in close contact with the trabecular bone. There was an absence of osteolysis, cysts, peri-implant fractures, or findings indicative of an adverse local reaction. Subjectively, limited differences in bone substitution were visualized between formulations at 3 weeks and 8 weeks for the TN-SM composition in FIG. 11A. TN-ISM composition was characterized by a decrease in radio-opacity of TN-ISM by 8-weeks as seen in FIG. 11B, suggestive of increased bone substitution.

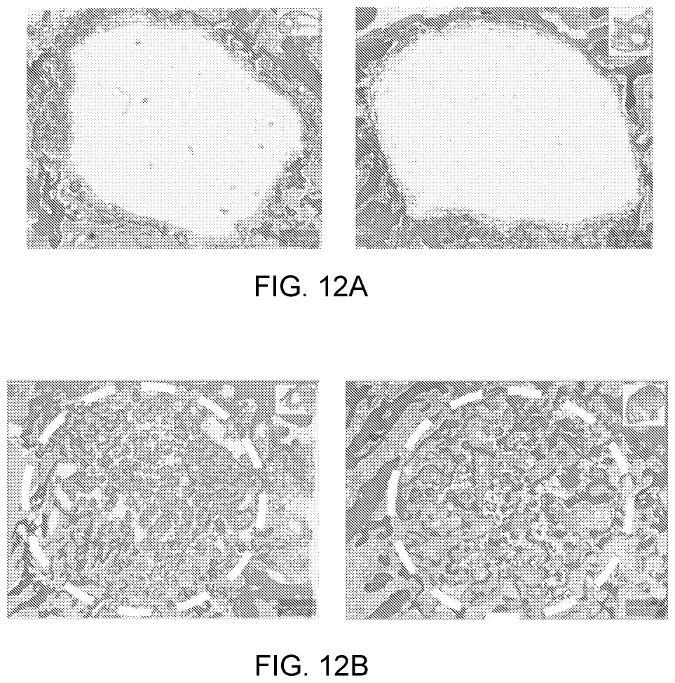

Representative histology images are shown in FIGS. 12A-12B. The 3-week cohorts were compared to the 8-week cohorts to assess composition/bone substitution. For all formulations, the composition was removed during the decalcification process for histology. An empty (white) void seen on the cross-section was indicative of the composition being present.

The TN-SM composition was characterized by minimal bone ingrowth at both timepoints. Most of the defect site was still occupied by the TN-SM composition by 8-weeks. The composition-bone interface was characterized by a thin lining of osteoid deposition.

The TN-ISM composition displayed increased eosinophilic trabecular bone and osteoid deposition that increased from 3 weeks to 8 weeks, characterized by increased osteoid and mineralized bone deposition that matched the staining intensity of native trabecular bone. The entirety of the defect site was now occupied by a combination of mature bone, osteoid, and active cellular infiltrate of osteoblastic nature, with moderate evidence of organized trabeculation. TN-ISM formulations had the presence of basophilic amorphous matter that was limited to the implantation site. Similar to the CBCT findings, there was an absence of osteolysis, cysts, or findings indicative of an adverse local reaction.

The addition of CaCO₃ nanoparticles resulted in increased porosity (as shown in Example 2 with Compositions 2 and 3). The increase in porosity facilitated cell infiltration and the formation of vascular networks to aid in composition turnover and bone substitution as clearly evidenced by the CBCT and histological findings. The bone trabecula at 8 weeks did not exactly resemble native adjacent trabecula. However, histological images at 8 weeks showed similar trabecular architecture at this timepoint; longer timepoints may result in additional bone substitution and trabecular maturity with increased mechanical strength.

### Example 6. In Vivo Evaluation of the Compositions Comprising CaCO₃ Nanoparticles in the Canine Critical Sized Defect for Implant Stabilization

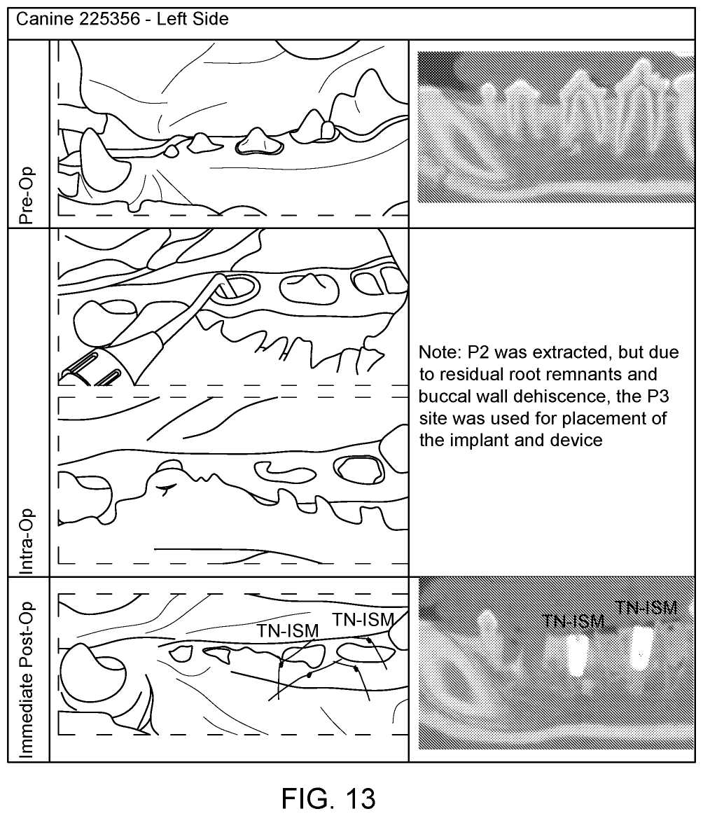

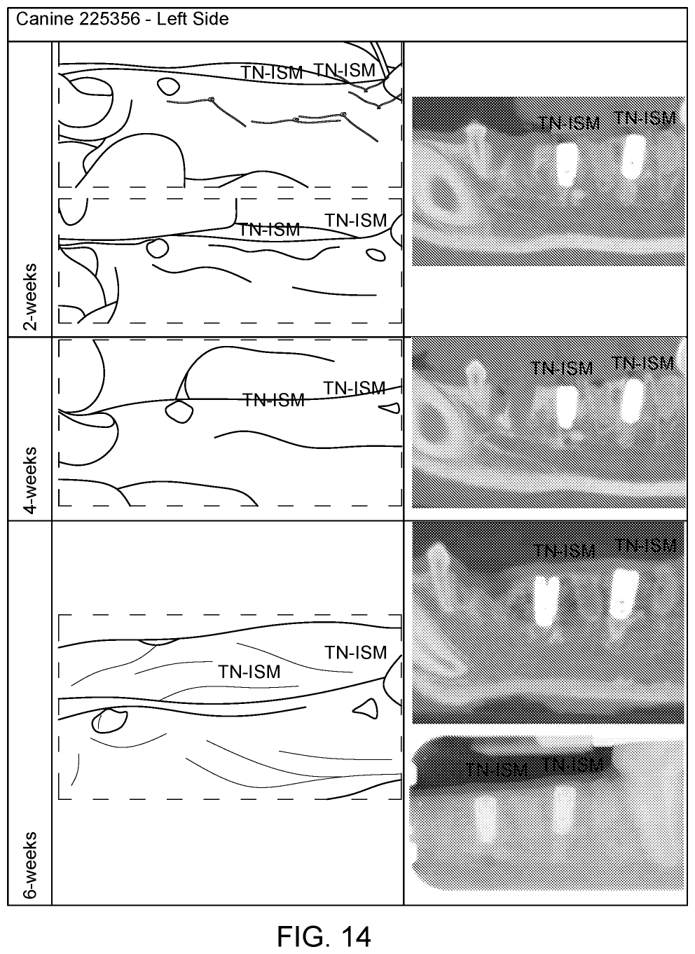

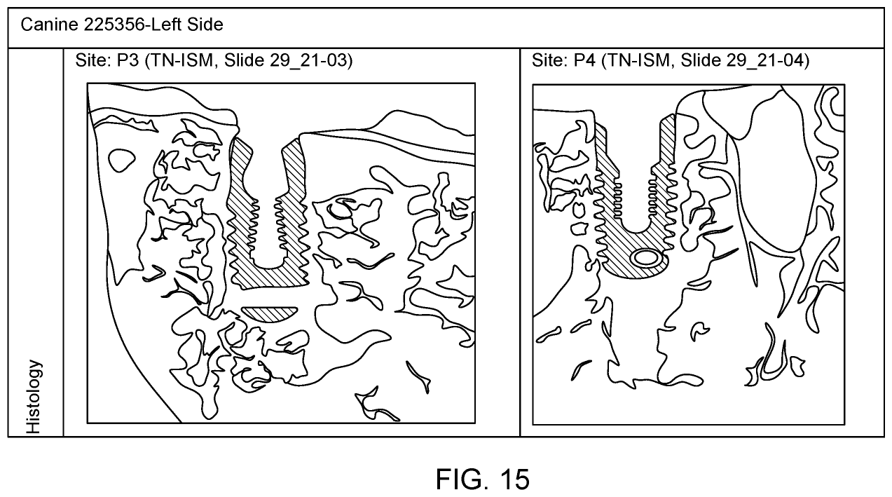

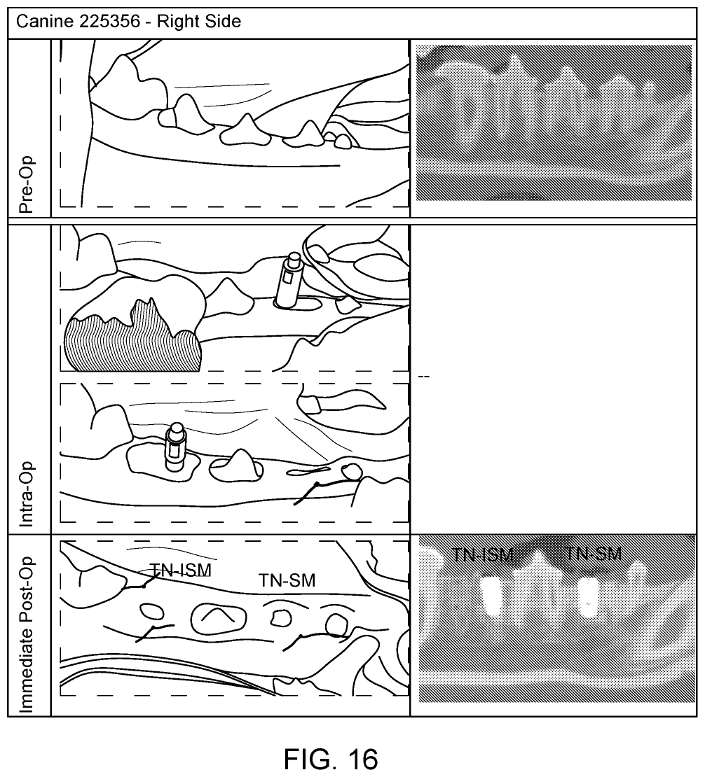

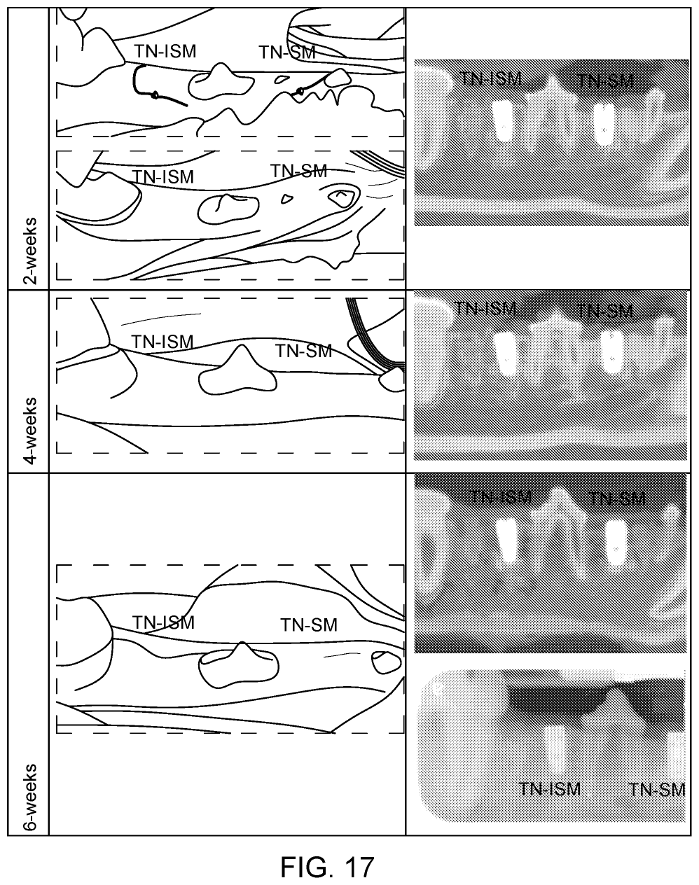

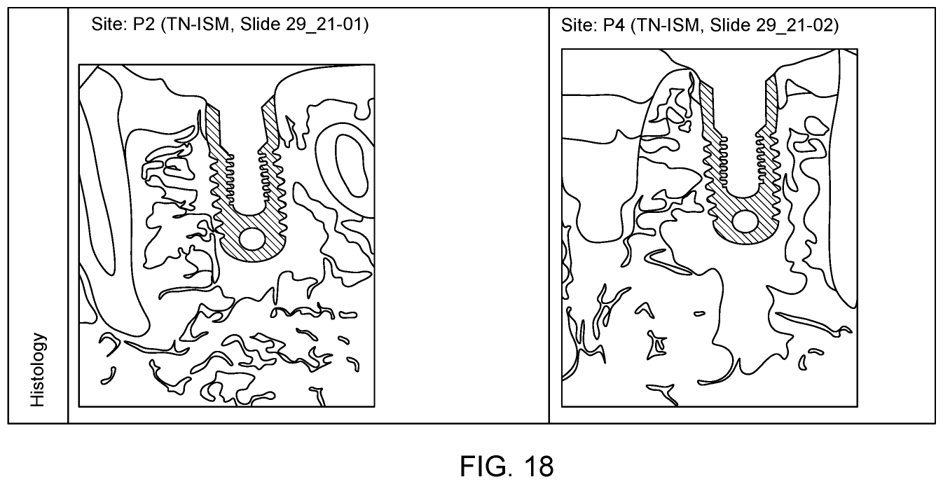

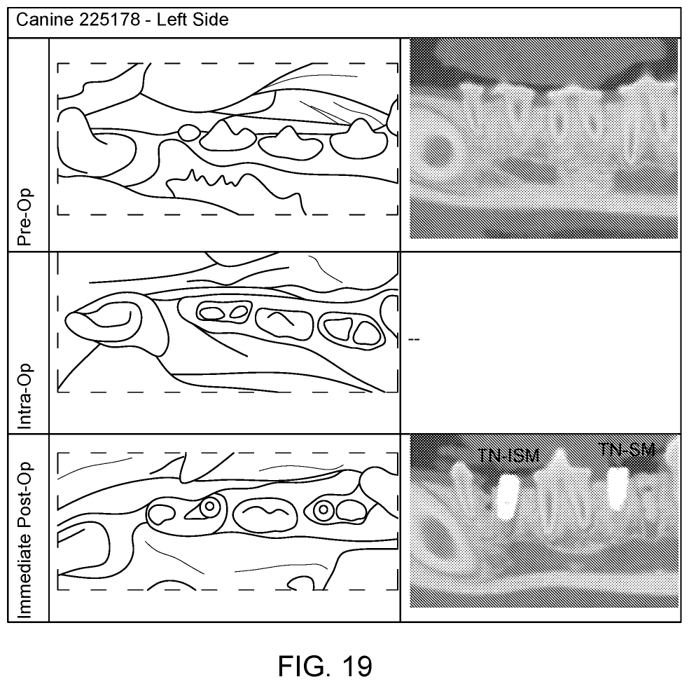

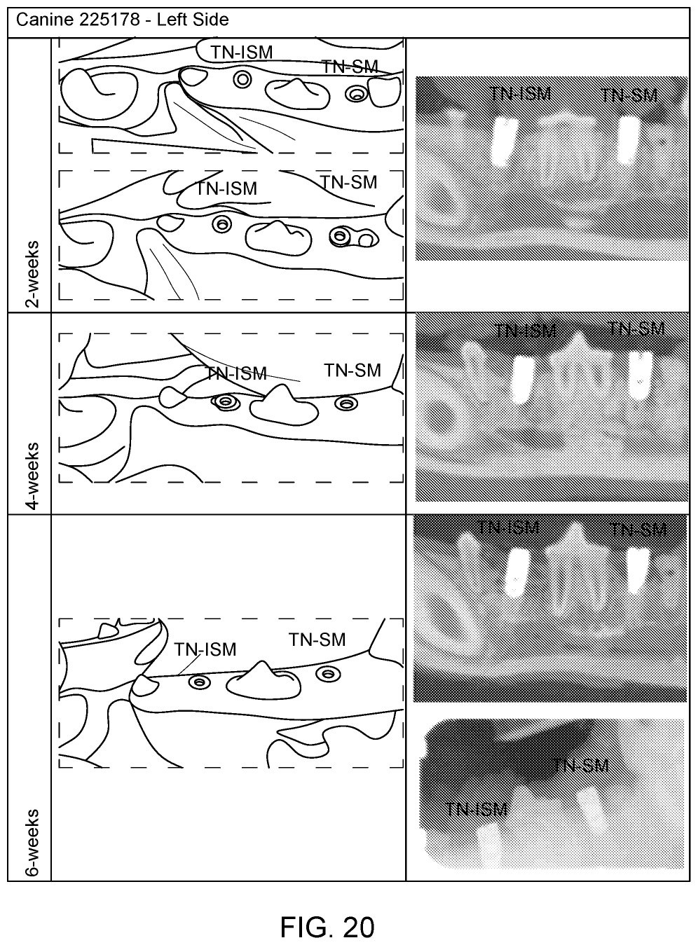

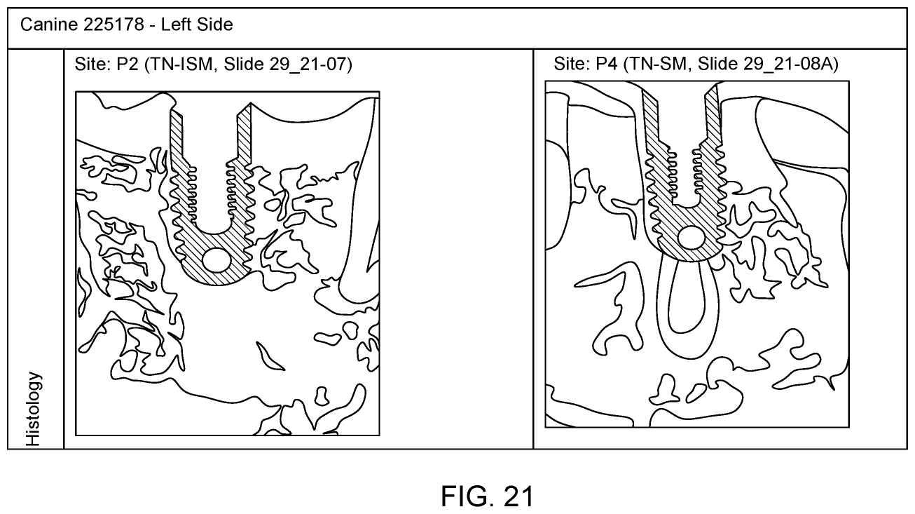

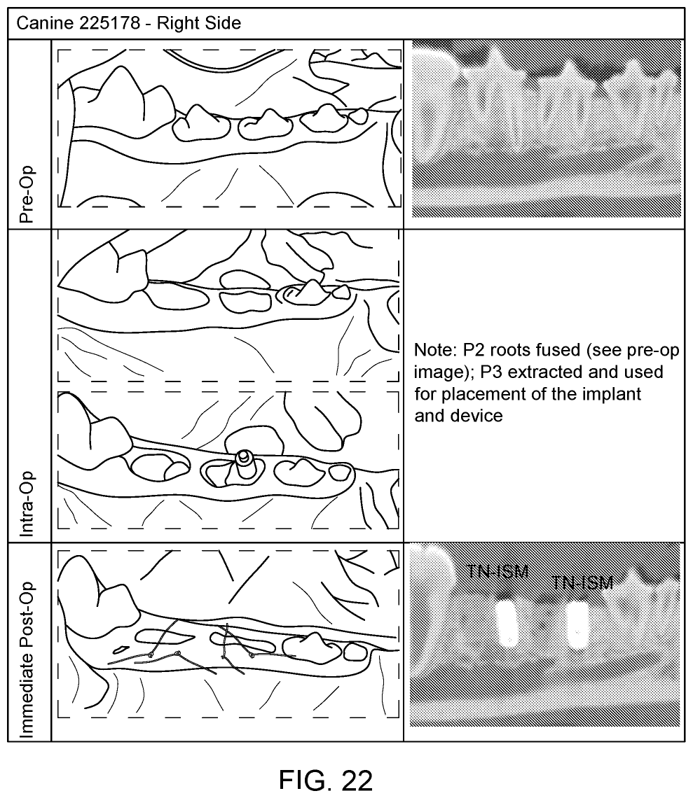

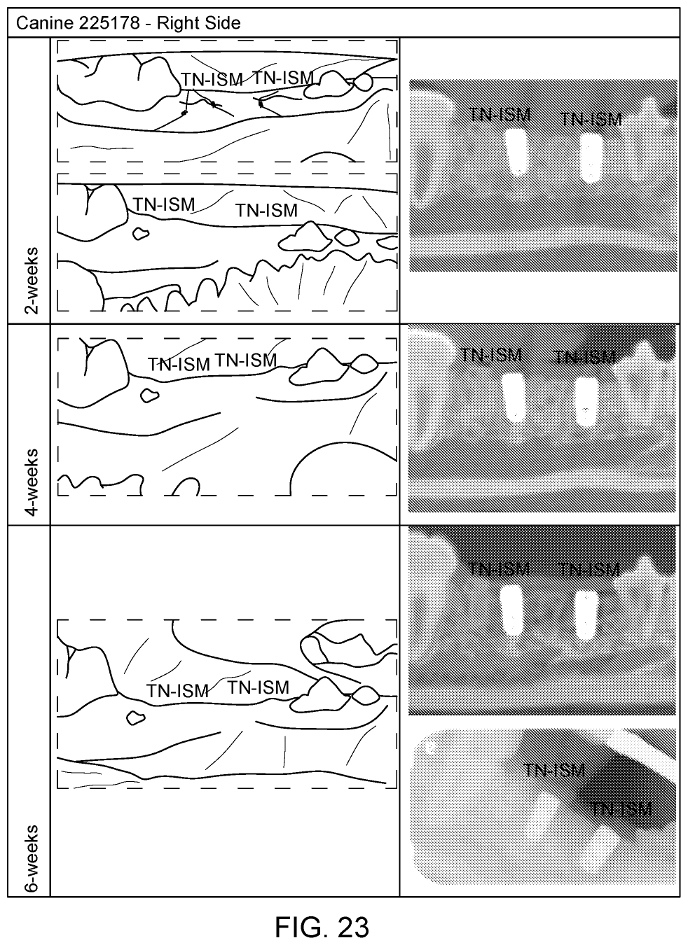

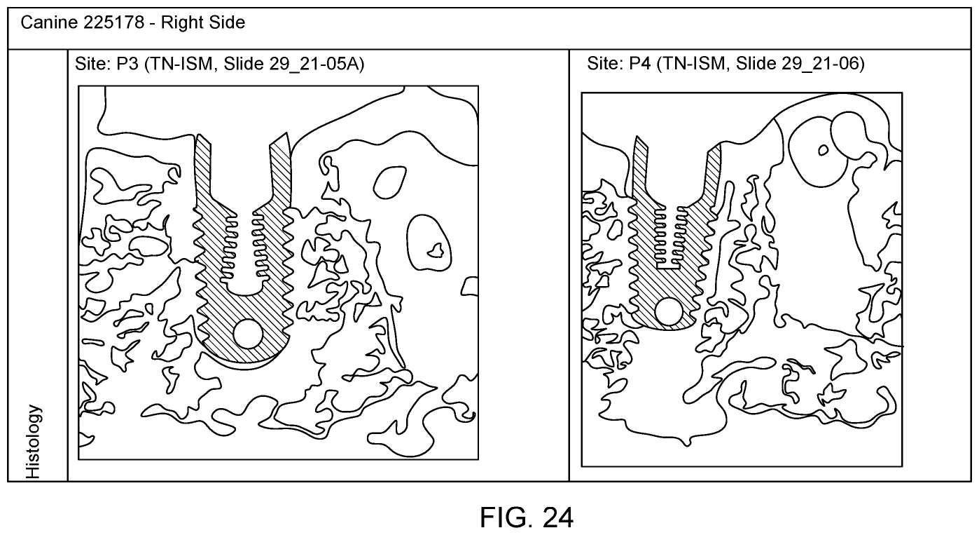

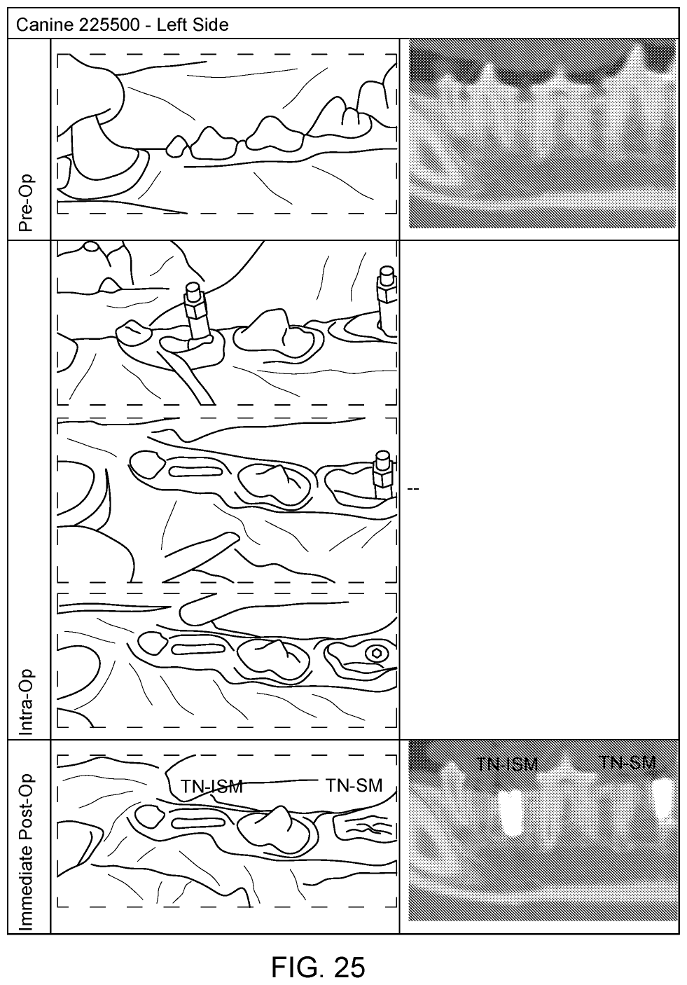

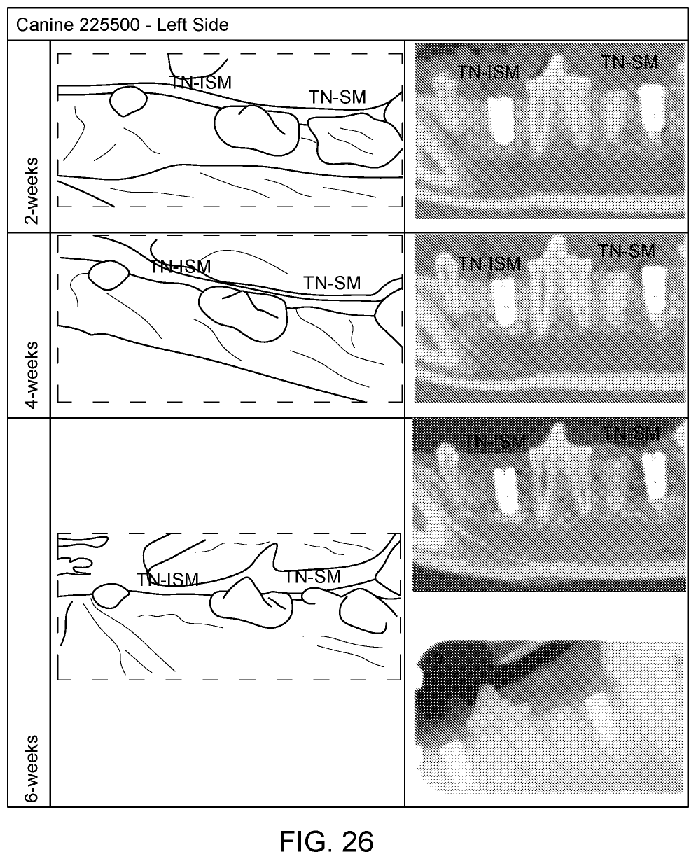

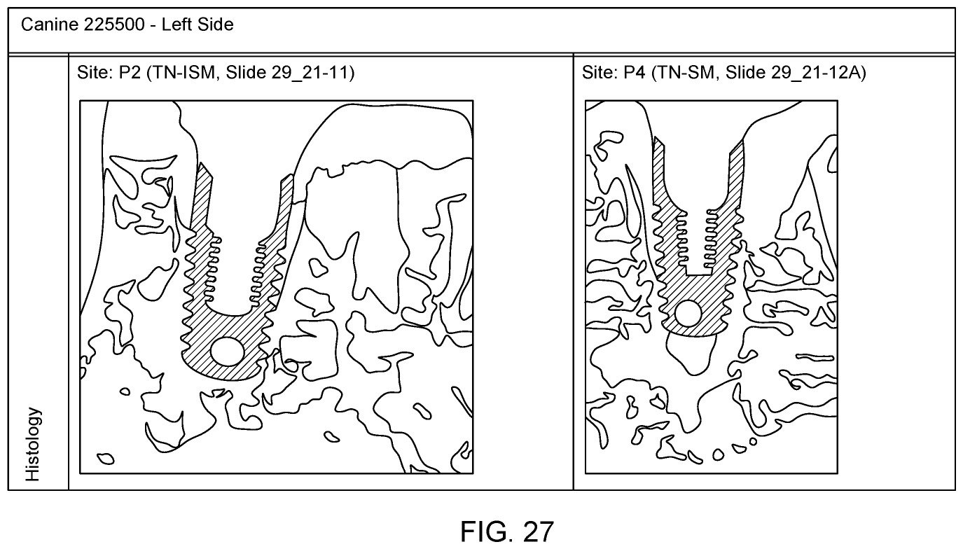

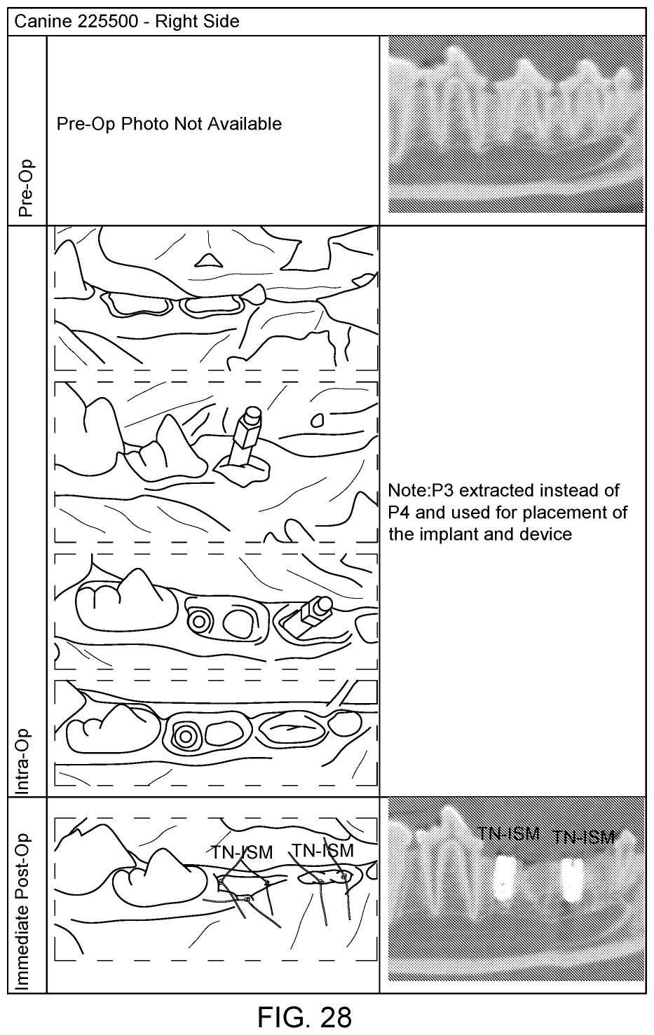

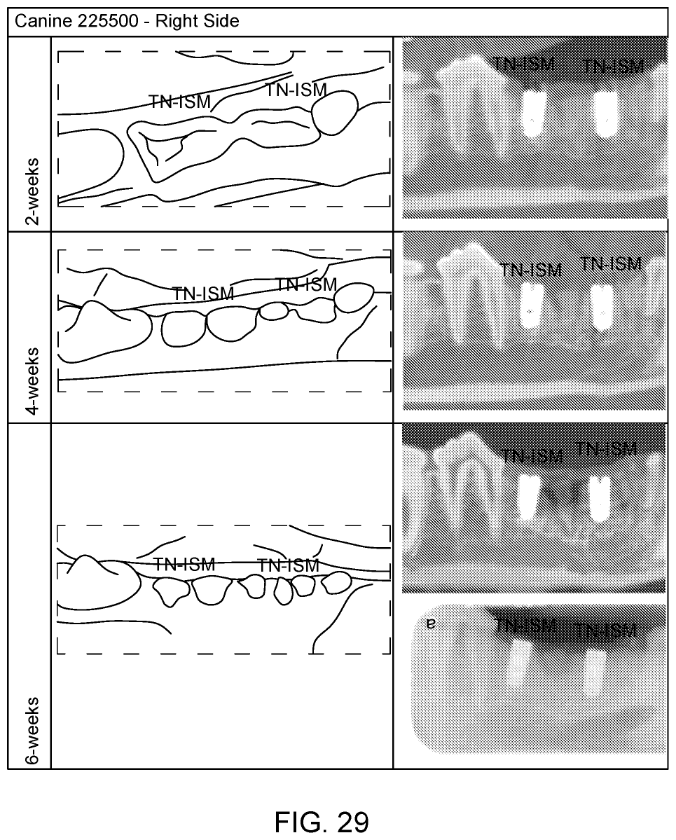

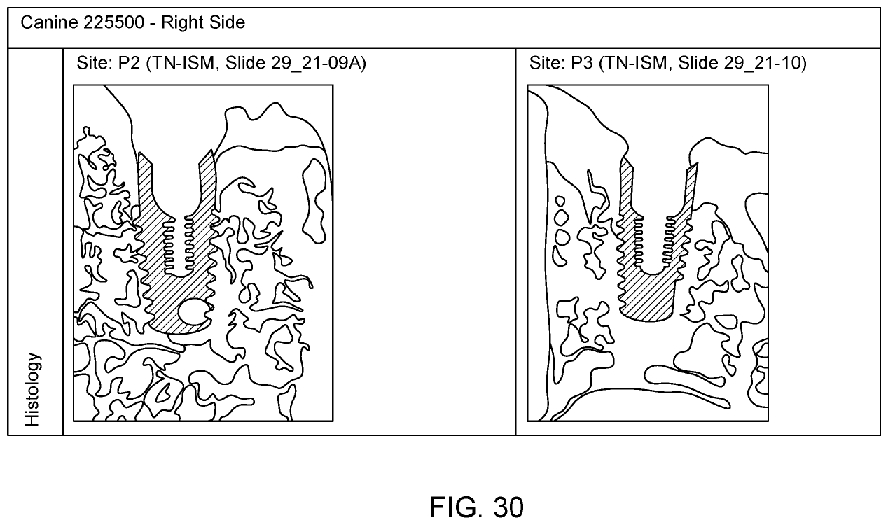

In this example, Compositions 14 and 15 were implanted into the mandibular defect of canines. Images, including clinical, CBCT, and histological of the implant procedure for three different canine patients are illustrated in FIGS. 13-30. Bilateral flapless extractions of the second and fourth mandibular premolar teeth were planned to allow for separation of the experimental sites and to minimize the healing burden during recovery. Upon completion of the extractions, the sites were prepared using the sides of an implant ostcotomy twist drill to remove the periodontal ligament (PDL) from the coronal ˜50% of each extraction socket. The adjacent empty socket designated not to receive a dental implant was treated in the same manner to prepare the site for test biomaterial deposit. Osteotomies were prepared in one root socket of each extracted tooth to receive a dental implant stabilized by the composition. The implant osteotomies were over-prepared with the next size twist drill wider than the size appropriate for the implant to be placed in a clinical situation. Prior to activation of the stabilization material, the implant underwent trial placement with an Osstell peg to assess stability by measurement of Resonance Frequency Analysis (RFA), referred to as Implant Stability Quotient (ISQ) when using the Osstell device, and to ensure that the site was properly prepared, i.e., it did not provide stability to the implant.

Care was taken to inject the compositions such that the apical ˜50% of the implant site was filled with the remaining material injected into the adjacent empty socket. A 3.7×8 mm diameter Zimmer implant (P/N: TSVB8) was placed with the implant platform at least level with the socket bone crest but, where possible, 1-2 mm below the bony alveolar ridge crest. Once the implant was placed, excess material was trimmed to 2-3 mm below the platform of the implant. After 15 minutes of cure time, Osstell ISQ readings were obtained and, once complete, a cover screw placed and soft tissues approximated without flap elevation. Clinical photographs were captured before, during and after the procedure. Soft tissue healing assessments as shown in Table 11 below and the CBCT's shown in FIGS. 13-30 were performed at 2, 4 and 6 weeks post-operatively.

TABLE 11
Modified Gingival Scoring Index

| Score | Description |
|---|---|
| 0 | Absence of inflammation: normal gingiva |
| 1 | Mild inflammation of a portion of the gingival unit: slight change in color, little change in |
| | texture |
| 2 | Mild inflammation of the entire gingival unit |
| 3 | Moderate inflammation of the entire gingival unit: moderate glazing, redness, edema and/or |
| | hypertrophy, of the entire gingival unit |
| 4 | Severe Inflammation of the entire gingival unit: marked redness and edema and/or |
| | hypertrophy, ulceration, spontaneous bleeding of the gingival unit |

Immediately prior to termination, clinical photographs of the implant sites were obtained. After euthanasia and necropsy, post-mortem CBCT's were captured, and the specimens placed in containers with gauze soaked with PBS solution. Within 6 hours of necropsy, the implant stability was measured through Osstell ISQ readings before testing for removal torque. Upon completion of these tests, the test sites were harvested from the hemi-mandibles for histology and placed in 10% neutral buffered formalin and sent for preparation of resin embedded nonmineralized histology slides.

TABLE 12
Summary of the Osstell Results

| | | | Termination |
|---|---|---|---|
| Formulation | Pre-TN | Post-TN | (6-weeks) |
| Composition 14 | 10 ± 4 | 69 ± 5 | 70 ± 5 |
| (TN-SM) | | | |
| Composition 15 | 16 ± 10 | 74 ± 5 | 71 ± 8 |
| (TN-ISM) | | | |

TABLE 13
Tabulated ISQ Data

| | | | Pre-TN | | | | | | Post-TN | | | | | | Termination (6-weeks) | | | | | |
|---|---|---|---|---|---|---|---|---|---|---|---|---|---|---|---|---|---|---|---|---|
| Canine | Site | Comp. # | Buccal | | | Mesial | | | Buccal | | | Mesial | | | Buccal | | | Mesial | | |
| 225356 | RP2 | Comp. # 14 | 7 | 7 | 7 | 7 | 10 | 7 | 75 | 75 | 75 | 75 | 75 | 75 | 75 | 75 | 75 | 75 | 75 | 75 |
| | | (TN-SM) | | | | | | | | | | | | | | | | | | |
| | RP4 | Comp. # 15 | 35 | 14 | 20 | 30 | 30 | 30 | 63 | 68 | 62 | 68 | 68 | 68 | 75 | 75 | 76 | 78 | 78 | 79 |
| | | (TN-ISM) | | | | | | | | | | | | | | | | | | |
| | LP3 | Comp. # 15 | 7 | 7 | 14 | 7 | 7 | 10 | 79 | 79 | 79 | 80 | 80 | 80 | 83 | 83 | 83 | 82 | 82 | 83 |
| | | (TN-ISM) | | | | | | | | | | | | | | | | | | |
| | LP4 | Comp. # 15 | 30 | 35 | 20 | 30 | 32 | 30 | 79 | 79 | 79 | 80 | 84 | 80 | 63 | 63 | 62 | 74 | 74 | 74 |
| | | (TN-ISM) | | | | | | | | | | | | | | | | | | |
| 225178 | RP3 | Comp. # 15 | 10 | 10 | 7 | 7 | 7 | 17 | 79 | 78 | 78 | 78 | 78 | 78 | 74 | 74 | 74 | 74 | 74 | 74 |
| | | (TN-ISM) | | | | | | | | | | | | | | | | | | |
| | RP4 | Comp. # 15 | 14 | 10 | 10 | 7 | 7 | 7 | 76 | 76 | 76 | 80 | 80 | 80 | 65 | 61 | 66 | 60 | 60 | 60 |
| | | (TN-ISM) | | | | | | | | | | | | | | | | | | |
| | LP2 | Comp. # 15 | 7 | 10 | 7 | 14 | 7 | 14 | 69 | 69 | 69 | 69 | 70 | 69 | 52 | 52 | 52 | 56 | 56 | 56 |
| | | (TN-ISM) | | | | | | | | | | | | | | | | | | |
| | LP4 | Comp. # 14 | 17 | 17 | 17 | 7 | 7 | 10 | 62 | 62 | 66 | 66 | 66 | 66 | 69 | 69 | 69 | 69 | 69 | 69 |
| | | (TN-SM) | | | | | | | | | | | | | | | | | | |
| 225500 | RP2 | Comp. # 15 | * | * | * | * | * | * | 70 | 70 | 70 | 72 | 72 | 72 | 73 | 73 | 72 | 72 | 72 | 72 |
| | | (TN-ISM) | | | | | | | | | | | | | | | | | | |
| | RP3 | Comp. # 15 | * | * | * | * | * | * | 71 | 71 | 72 | 69 | 70 | 69 | 69 | 71 | 71 | 70 | 70 | 70 |
| | | (TN-ISM) | | | | | | | | | | | | | | | | | | |
| | LP2 | Comp. # 15 | * | * | * | * | * | * | 70 | 78 | 79 | 77 | 77 | 77 | 76 | 76 | 76 | 75 | 75 | 75 |
| | | (TN-ISM) | | | | | | | | | | | | | | | | | | |
| | LP4 | Comp. # 14 | * | * | * | * | * | * | 63 | 63 | 64 | 69 | 69 | 68 | 68 | 68 | 68 | 65 | 65 | 65 |
| | | (TN-SM) | | | | | | | | | | | | | | | | | | |

* indicates floating implant, Osstell results were not recorded because the implant was withdrawn from the site by magnetic attraction of the Osstell wand.

### Removal Torque Results

TABLE 14
6-week Reverse Torque Testing Summary Results

| | Reverse |
|---|---|
| Formulation | Torque (N-cm) |
| Composition 14 | 18.1 ± 5.1 |
| (TN-SM) | |
| Composition 15 | 23.1 ± 7.2 |
| (TN-ISM) | |

### Radiographic Findings

Qualitative assessment of the periapical radiographs revealed significant bone loss at two implant stabilization sites: canine 225178, left posterior site and canine 225500, left posterior site. Both of the sites were TN-SM sites, and the vertical bone loss pattern was consistent with failures observed in similar human clinical situations. None of the TN-ISM sites exhibited comparable levels of bone loss. TN-ISM sites that did exhibit bone loss had significantly less loss of coronal bone.

### Clinical Findings

The oral health of each of the implant sites was evaluated at each follow-up at 2-, 4- and 6-weeks. The results from each of the three study subjects is located in Tables 15-17.

TABLE 15
Canine 225356 Oral Health Scores
Canine 225356

| | |
|---|---|
| Left | Right |

| Canine | Anterior | Posterior | Anterior | Posterior |
|---|---|---|---|---|
| 225356 | TN-ISM | TN-ISM | TN-SM | TN-ISM |
| 2-week | 2 | 2 | 3 | 2 |
| 4-week | 1 | 2 | 2 | 3 |
| 6-week | 0 | 1 | 0 | 0 |

TABLE 16
Canine 225178 Oral Health Scores
Canine 225178

| | |
|---|---|
| Left | Right |

| Canine | Anterior | Posterior | Anterior | Posterior |
|---|---|---|---|---|
| 225178 | TN-ISM | TN-SM | TN-ISM | TN-ISM |
| 2-week | 3 | 2 | 3 | 1 |
| 4-week | 1 | 1 | 0 | 0 |
| 6-week | 1 | 3 | 1 | 3 |

TABLE 17
Canine 225500 Oral Health Scores
Canine 225500

| | |
|---|---|
| Left | Right |

| Canine | Anterior | Posterior | Anterior | Posterior |
|---|---|---|---|---|
| 225500 | TN-ISM | TN-SM | TN-ISM | TN-ISM |
| 2-week | 0 | 3 | 3 | 3 |
| 4-week | 0 | 2 | 0 | 1 |
| 6-week | 0 | 1 | 0 | 2 |

### Histology Findings

The histological examination of the experimental sites was performed on en bloc harvested tissues which were prepared in sagittal plane as non-decalcified, resin embedded specimens and stained with van Gieson's blue and acid picro fuchsin. All slides were prepared in the sagittal plane. FIGS. 13-30 illustrate the 6-week post-operative histological findings at sites of TN-ISM stabilization of dental implants, TN-SM stabilization of dental implants and immediately placed following tooth extraction.

At the six-week time point, the stabilization material is evident as a heterogeneous granulated tan to brown substance. The highly porous TN-ISM is punctuated by round, white fields originally occupied by carbon dioxide gas. In most images the stabilization material is present immediately surrounding the implant and filling the confines of the implant osteotomy, both laterally and apically. In some of the images the material is also seen filling contiguous trabecular spaces.

Qualitatively, the TN-ISM formulation substance appeared to be invaded by osseous tissues to a significantly greater extent at the six-week time point than the TN-SM. In fact, total replacement of the TN-ISM layer was nearly complete at several stabilization site regions.

The results presented confirm that composition 15 (TN-ISM) had superior bone regeneration and resorption rate compared to the composition 14 (TN-SM). The inclusion of calcium carbonate nanoparticles into the formulation resulted in increased porosity of the composition which resulted in faster rate of bone regeneration.

### Example 7. Effects of the Acidity Modulating Additives on the pH and Adhesive Shear of Composition

This in vitro study evaluated the effects of the acidity modulating additives to the compositions. The compositions primarily included a multivalent metal phosphate salt mixed with phosphoserine. This mixture resulted in an acidic biomaterial when the components were mixed with water. To modulate the acidic nature of the composition, several additives were chosen and added to the composition as shown in Table 18.

TABLE 18
List of compositions which include pH adjusting additives

| | Dry Components | | | | | | |
|---|---|---|---|---|---|---|---|
| | Multivalent Metal Salt | | Multidentate Organic Acid | | | | |
| | | mass (g) | | mass (g) | | | |
| | | (mean | | (mean | Liquid Component | | Mixing and |
| | | particle | | particle | Aqueous | Volume | Application |
| Composition | Compound | size, μm)¹ | Compound | size, μm)¹ | Medium | (μL) | Technique |
| Composition 17 | Multiphasic | 0.738 | O-Phospho- | 0.462 | Water | 258 | Technique 1 |
| | calcium | 33.62 | L-serine | 9.9 | | | |
| | phosphate | | | | | | |
| | (NB019-54) | | | | | | |
| | Magnesium | 0.0048 | | | | | |
| | oxide | 9.33 | | | | | |
| Composition 18 | Multiphasic | 0.738 | O-Phospho- | 0.462 | Water | 268 | Technique 1 |
| | calcium | 33.62 | L-serine | 9.9 | | | |
| | phosphate | | | | | | |
| | (NB019-54) | | | | | | |
| | Magnesium | 0.0097 | | | | | |
| | oxide | 9.33 | | | | | |
| Composition 19a | Multiphasic | 0.738 | O-Phospho- | 0.462 | Water | 280 | Technique 1 |
| | calcium | 33.62 | L-serine | 9.9 | | | |
| | phosphate | | | | | | |
| | (NB019-54) | | | | | | |
| | Magnesium | 0.0193 | | | | | |
| | oxide | 9.33 | | | | | |
| Composition 19b | Multiphasic | 1.476 | O-Phospho- | 0.924 | Water | 560 | Technique 1 |
| | calcium | 33.62 | L-serine | 9.9 | | | |
| | phosphate | | | | | | |
| | (NB019-54) | | | | | | |
| | Magnesium | 0.039 | | | | | |
| | oxide | 9.33 | | | | | |
| Composition 20 | Multiphasic | 0.738 | O-Phospho- | 0.46 | Water | 258 | Technique 1 |
| | calcium | 33.62 | L-serine | 9.9 | | | |
| | phosphate | | | | | | |
| | (NB019-54) | | | | | | |
| | Calcium | 0.0139 | | | | | |
| | silicates2 | 6.8 | | | | | |
| Composition 21 | Multiphasic | 0.738 | O-Phospho- | 0.46 | Water | 268 | Technique 1 |
| | calcium | 33.62 | L-serine | 9.9 | | | |
| | phosphate | | | | | | |
| | (NB019-54) | | | | | | |
| | Calcium | 0.0278 | | | | | |
| | silicates2 | 6.8 | | | | | |
| Composition 22 | Multiphasic | 0.738 | O-Phospho- | 0.46 | Water | 280 | Technique 1 |
| | calcium | 33.62 | L-serine | 9.9 | | | |
| | phosphate | | | | | | |
| | (NB019-54) | | | | | | |
| | Calcium | 0.042 | | | | | |
| | silicates2 | 6.8 | | | | | |
| Composition 23a | Multiphasic | 0.738 | O-Phospho- | 0.46 | Water | 285 | Technique 1 |
| | calcium | 33.62 | L-serine | 9.9 | | | |
| | phosphate | | | | | | |
| | (NB019-54) | | | | | | |
| | Calcium | 0.056 | | | | | |
| | silicates2 | 6.8 | | | | | |
| Composition 23b | Multiphasic | 1.476 | O-Phospho- | 0.924 | Water | 570 | Technique 1 |
| | calcium | 33.62 | L-serine | 9.9 | | | |
| | phosphate | | | | | | |
| | (NB019-54) | | | | | | |
| | Calcium | 0.056 | | | | | |
| | silicates2 | 6.8 | | | | | |
| Composition 24 | Multiphasic | 0.738 | O-Phospho- | 0.46 | Water | 258 | Technique 1 |
| | calcium | 33.62 | L-serine | 9.9 | | | |
| | phosphate | | | | | | |
| | (NB019-54) | | | | | | |
| | Calcium | 0.0115 | | | | | |
| | Citrate | 12.5 | | | | | |
| | tetrahydrate | | | | | | |
| Composition 25 | Multiphasic | 0.738 | O-Phospho- | 0.46 | Water | 268 | Technique 1 |
| | calcium | 33.62 | L-serine | 9.9 | | | |
| | phosphate | | | | | | |
| | (NB019-54) | | | | | | |
| | Calcium | 0.023 | | | | | |
| | Citrate | 6.8 | | | | | |
| | tetrahydrate | | | | | | |
| Composition 26a | Multiphasic | 0.738 | O-Phospho- | 0.46 | Water | 280 | Technique 1 |
| | calcium | 33.62 | L-serine | 9.9 | | | |
| | phosphate | | | | | | |
| | (NB019-54) | | | | | | |
| | Calcium | 0.046 | | | | | |
| | Citrate | 6.8 | | | | | |
| | tetrahydrate | | | | | | |
| Composition 26b | Multiphasic | 0.738 | O-Phospho- | 0.46 | Water | 560 | Technique 1 |
| | calcium | 33.62 | L-serine | 9.9 | | | |
| | phosphate | | | | | | |
| | (NB019-54) | | | | | | |
| | Calcium | 0.092 | | | | | |
| | Citrate | 6.8 | | | | | |
| | tetrahydrate | | | | | | |
| Composition 27 | Multiphasic | 0.738 | O-Phospho- | 0.46 | 0.1M | 258 | Technique 1 |
| | calcium | 33.62 | L-serine | 9.9 | NaOH | | |
| | phosphate | | | | | | |
| | (NB019-54) | | | | | | |
| Composition 28 | Multiphasic | 0.738 | O-Phospho- | 0.46 | 0.4M | 258 | Technique 1 |
| | calcium | 33.62 | L-serine | 9.9 | NaOH | | |
| | phosphate | | | | | | |
| | (NB019-54) | | | | | | |
| Composition 29a | Multiphasic | 0.738 | O-Phospho- | 0.46 | 3.2M | 258 | Technique 1 |
| | calcium | 33.62 | L-serine | 9.9 | NaOH | | |
| | phosphate | | | | | | |
| | (NB019-54) | | | | | | |
| Composition 29b | Multiphasic | 1.476 | O-Phospho- | 0.924 | 3.2M | 560 | Technique 1 |
| | calcium | 33.62 | L-serine | 9.9 | NaOH | | |
| | phosphate | | | | | | |
| | (NB019-54) | | | | | | |
| Composition 30a | Multiphasic | 0.738 | O-Phospho- | 0.46 | Water | 258 | Technique 1 |
| | calcium | 33.62 | L-serine | 9.9 | | | |
| | phosphate | | | | | | |
| | (NB019-54) | | | | | | |
| Composition 30b | Multiphasic | 1.476 | O-Phospho- | 0.924 | Water | 560 | Technique 1 |
| | calcium | 33.62 | L-serine | 9.9 | | | |
| | phosphate | 22.84 | | 9.9 | | | |
| | (NB019-54) | | | | | | |
| | Calcium | 0.087 | | | | | |
| | carbonate | 0.015-0.19 | | | | | |

### Method

The compositions were mixed for 30 seconds and formed into disks (approximately 10 mm diameter×5 mm high) and were placed in 0.001 M modified saline solution in a ratio of 1 g of composition to 5 mL of 0.001 M modified saline. Samples were prepared using a cylindrical mold made from polyethylene and placed into the modified saline solution before 2 minutes post product activation had elapsed. The samples were removed at 15 minutes and punched out of the mold before being replaced in the solution for the remainder of the incubation time. The elute of each composition was collected and tested for pH, as detailed in Example 1. The compositions were also assessed for the adhesive strength using the method described in Example 1.

### pH of the Elute

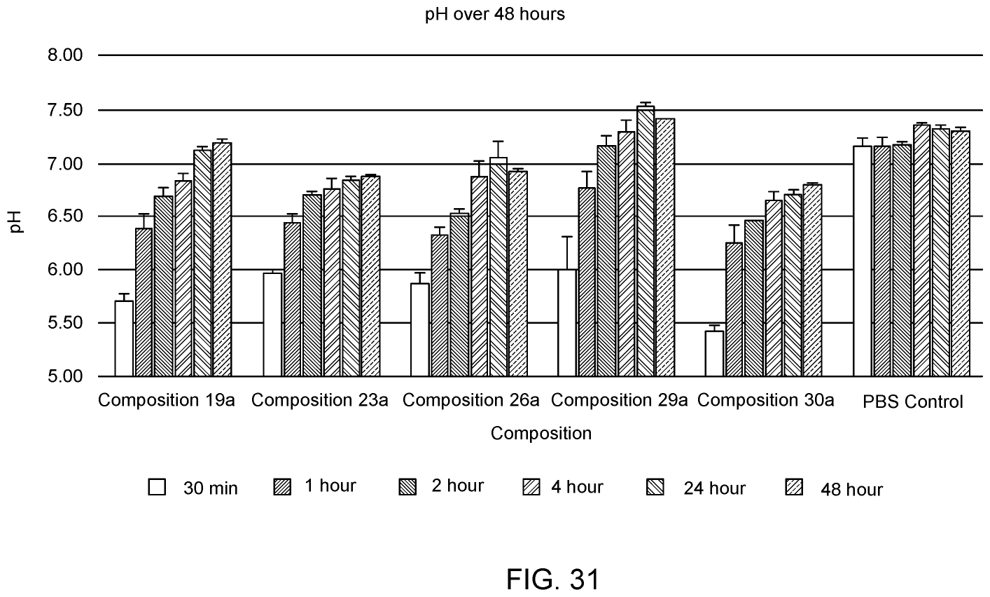

The pH of the elute for all the compositions, illustrated for Compositions 19a, 23a, 26a, 29a, and 30a against a PBS control in FIG. 31, showed a similar trend where the pH becomes neutral within the first 24 hours. Based on the pH, all the compositions can be explored as pH modulating additives into the composition.

### Adhesive Shear

To assess whether incorporation of the additives influence the adhesive properties of the composition, adhesive shear testing was performed using a similar method the that described in Example 1. Table 19 shows the resultant adhesive shear data for Compositions 19a, 23a, 26a, 29a, and 30a.

TABLE 19
Adhesive Shear data for acidity modulation compositions

| | Adhesive |
|---|---|
| Composition | Shear (MPa) |
| Composition 19b | 2.4 |
| Composition 23b | 3.1 |
| Composition 26b | 2.9 |
| Composition 29b | 3.2 |
| Composition 30b | 2.3 |

### Example 8: Influence of Porosity on the Physical State of the Cured Composition

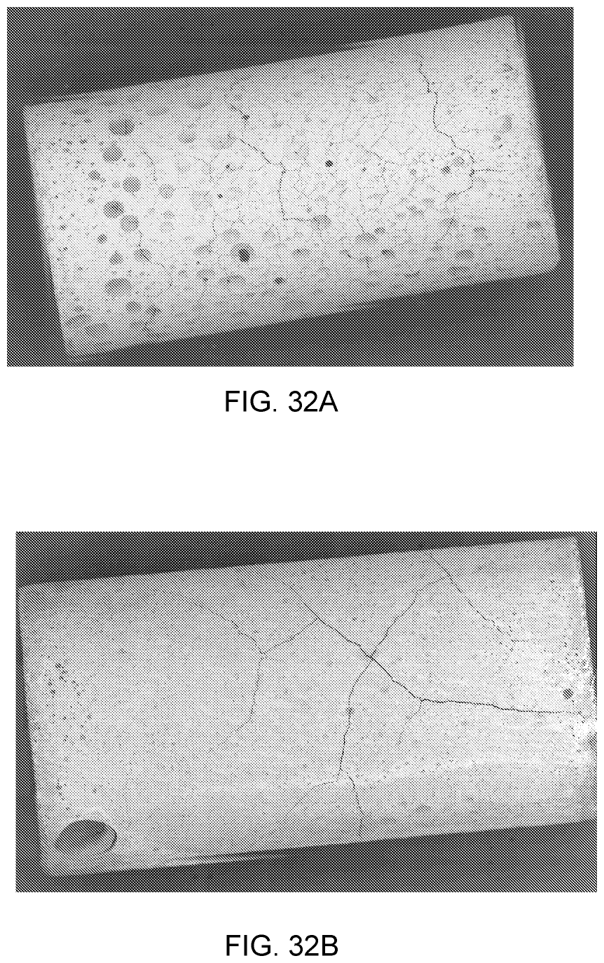

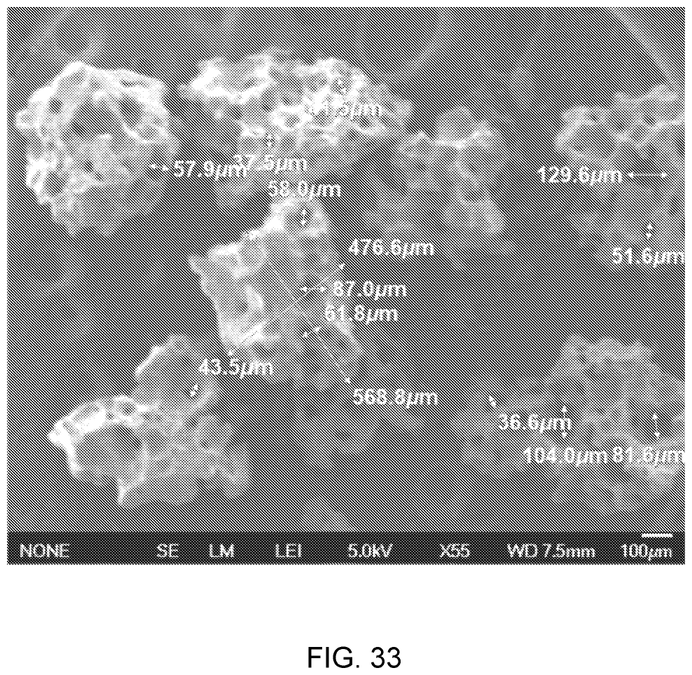

The product of the curing reaction involving incorporating a pH adjusting agent, such as calcium carbonate, generated a more porous bulk material, as shown in FIG. 32A which is 3D reconstruction of micro-CT data. The denser cylinder illustrated in FIG. 32B was produced by the TN-SM composition listed in Table 3, whereas the porous cylinder illustrated in FIG. 1A was produced using the TN-ISM composition listed in Table 3. FIG. 33 illustrates the abundance of pores in the 40-120 μm range present throughout the porous solid illustrated in FIG. 1A, i.e., the TN-ISM composition, as seen in a granulated form of the material.

Total porosity as well as the breakdown of the porosity levels between various size thresholds from Mercury Intrusion Porosimetry comparing cured plugs of the TN-SM composition to the TN-ISM composition are presented in Table 20. In addition, total porosity data from micro-CT for several adhesive compositions after cured plugs were created is presented in Table 21.

TABLE 20
Total Porosity and Breakdown of Pore Size Distribution
Obtained via Mercury Intrusion Porosimetry

| | Composition 14 | Composition 15 |
|---|---|---|
| | (TN-SM) | (TN-ISM) |
| Overall Porosity | 12% ± 0.5 | 38% ± 1.2 |

| | | | |
|---|---|---|---|
| 0-20 | μm | 12% ± 0.6 | 25% ± 2.2 |
| 20-40 | μm | 0.3% ± 0.1 | 7% ± 0.8 |
| 40-60 | μm | 0.1% ± 0.03 | 2% ± 1.2 |
| 60-100 | μm | 0.2% ± 0.03 | 2% ± 0.5 |
| 100-250 | μm | 0.2% ± 0.1 | 1% ± 0.7 |

TABLE 21
Total Porosity of Various Adhesive Compositions by micro-CT analysis
% Porosity

| | Compo- | Compo- | Compo- | Compo- | Compo- | | Composition | Composition | | | |
|---|---|---|---|---|---|---|---|---|---|---|---|
| | sition | sition | sition | sition | sition | Composition | 35 | 36 | Composition | Composition | Composition |
| | 14 | 15 | 16 | 31 | 33 | 34 | Granule | Granule | 37 | 38 | 39 |
| Time | TN-SM | TN-ISM | TN-CFF | TOM750 | ADBS I | ADBS II | 500-710 | 710-1000 | (TN-FF) | (TN-FF 2) | (TN-VAF) |
| T = 0 | 3.0 | 28.7 | 4.1 | 29.8 | 24.9 | 24.4 | 58.5 | 62.2 | 38.6 | — | 4.1 |
| T = 2 | 7.1 | 37.7 | 4.4 | 44.7 | 36.6 | 29.4 | 58.3 | 65.8 | 27.4 | — | 2.7 |
| wks | | | | | | | | | | | |

### Example 9. Theoretical Maximum Porosity Yields from Inclusion of Calcium Carbonate in Existing Formulations

This example illustrates model calculations for the theoretical porosity of compositions including calcium carbonate. The following assumptions were made in the model calculations: 1. that the carbonate is fully converted to CO₂ gas; 2. that the CO₂ gas is fully expanded to atmospheric pressure; and 3. that 100% of the gas is retained within the reaction mixture and the cured solid, i.e., that no gas escapes the substance of the composition. Empirical results shown in Table 22 indicated that the volume of the total porosity is approximately 44% of the theoretical maximum when CaCO₃ nanoparticles are combined with the TOM250 formulation and mechanically mixed in a triturator capsule.

TABLE 22
Model calculations for the theoretical porosity of compositions including calcium carbonate

| Percentage | | Mass of | Moles of | Potential | Potential | Potential | | Percentage |
|---|---|---|---|---|---|---|---|---|
| w/w | Mass | Potentially | Potentially | Volume | Volume | Maximum | Exp. | Theoretical |
| CaCO₃ of | CaCO₃ | Available | Available | CO₂ gas | CO₂ gas | CO₂ gas | Observed | Amount of |
| Solids | (g) | CO₂ (g) | CO₂ | (L) | (ml) | Porosity | Porosity | CO₂ |
| 0.01% | 6.500E−05 | 2.858E−05 | 6.494E−07 | 1.455E−05 | 1.455E−02 | 2.9% | | |
| 0.02% | 1.300E−04 | 5.716E−05 | 1.299E−06 | 2.909E−05 | 2.909E−02 | 5.6% | | |
| 0.05% | 3.250E−04 | 1.429E−04 | 3.247E−06 | 7.274E−05 | 7.274E−02 | 13.0% | | |
| 0.10% | 6.500E−04 | 2.858E−04 | 6.494E−06 | 1.455E−04 | 1.455E−01 | 23.0% | | |
| 0.20% | 1.300E−03 | 5.716E−04 | 1.299E−05 | 2.909E−04 | 2.909E−01 | 37.4% | | |
| 0.50% | 3.250E−03 | 1.429E−03 | 3.247E−05 | 7.274E−04 | 7.274E−01 | 59.9% | | |
| 1.00% | 6.500E−03 | 2.858E−03 | 6.494E−05 | 1.455E−03 | 1.455E+00 | 74.9% | | |
| 2.00% | 1.300E−02 | 5.716E−03 | 1.299E−04 | 2.909E−03 | 2.909E+00 | 85.6% | 38.0% | 44.4% |
| 5.00% | 3.250E−02 | 1.429E−02 | 3.247E−04 | 7.274E−03 | 7.274E+00 | 93.7% | | |
| 10.00% | 6.500E−02 | 2.858E−02 | 6.494E−04 | 1.455E−02 | 1.455E+01 | 96.8% | | |

### Example 10. Theoretical Maximum Pore Size Resulting from the Inclusion of Calcium Carbonate Powder within Existing Formulations

This example illustrates model calculations for the theoretical maximum pore size of compositions including calcium carbonate. The following assumptions were made: 1. that the CaCO₃ particles were nonporous and spherical; 2. that the carbonate is 100% converted to CO₂ gas; 3. that the CO₂ gas is 100% expanded to atmospheric pressure, and 4. that 100% of the gas is retained within the composition substance. Empirical results shown in Table 23.

TABLE 23
Model calculations for the theoretical maximum pore size of compositions including calcium carbonate

| | | | | | Equivalent | Bubble | Bubble | Bubble |
|---|---|---|---|---|---|---|---|---|
| Grain size | Volume | Volume | Mass | | volume CO₂ | volume | size | size |
| (nm ø) | (nm³) | (cm³) | (g) | Moles | gas (L) | (μL) | (nm ø) | (μm ø) |
| 10 | 5.236E+02 | 5.236E−19 | 1.419E−18 | 1.418E−20 | 3.176E−19 | 3.176E−13 | 8.466E−02 | 8.466E−05 |
| 20 | 4.189E+03 | 4.189E−18 | 1.135E−17 | 1.134E−19 | 2.541E−18 | 2.541E−12 | 1.693E−01 | 1.693E−04 |
| 50 | 6.545E+04 | 6.545E−17 | 1.774E−16 | 1.772E−18 | 3.970E−17 | 3.970E−11 | 4.233E−01 | 4.233E−04 |
| 100 | 5.236E+05 | 5.236E−16 | 1.419E−15 | 1.418E−17 | 3.176E−16 | 3.176E−10 | 8.465E−01 | 8.465E−04 |
| 200 | 4.189E+06 | 4.189E−15 | 1.135E−14 | 1.134E−16 | 2.541E−15 | 2.541E−09 | 1.693E+00 | 1.693E−03 |
| 500 | 6.545E+07 | 6.545E−14 | 1.774E−13 | 1.772E−15 | 3.970E−14 | 3.970E−08 | 4.233E+00 | 4.233E−03 |
| 1000 | 5.236E+08 | 5.236E−13 | 1.419E−12 | 1.418E−14 | 3.176E−13 | 3.176E−07 | 8.465E+00 | 8.465E−03 |
| 2000 | 4.189E+09 | 4.189E−12 | 1.135E−11 | 1.134E−13 | 2.541E−12 | 2.541E−06 | 1.693E+01 | 1.693E−02 |
| 5000 | 6.545E+10 | 6.545E−11 | 1.774E−10 | 1.772E−12 | 3.970E−11 | 3.970E−05 | 4.233E+01 | 4.233E−02 |
| 10000 | 5.236E+11 | 5.236E−10 | 1.419E−09 | 1.418E−11 | 3.176E−10 | 3.176E−04 | 8.465E+01 | 8.465E−02 |

### Example 11. Influence of Method on pH of the Immediate Aqueous Environment Surrounding the Reaction Mixture

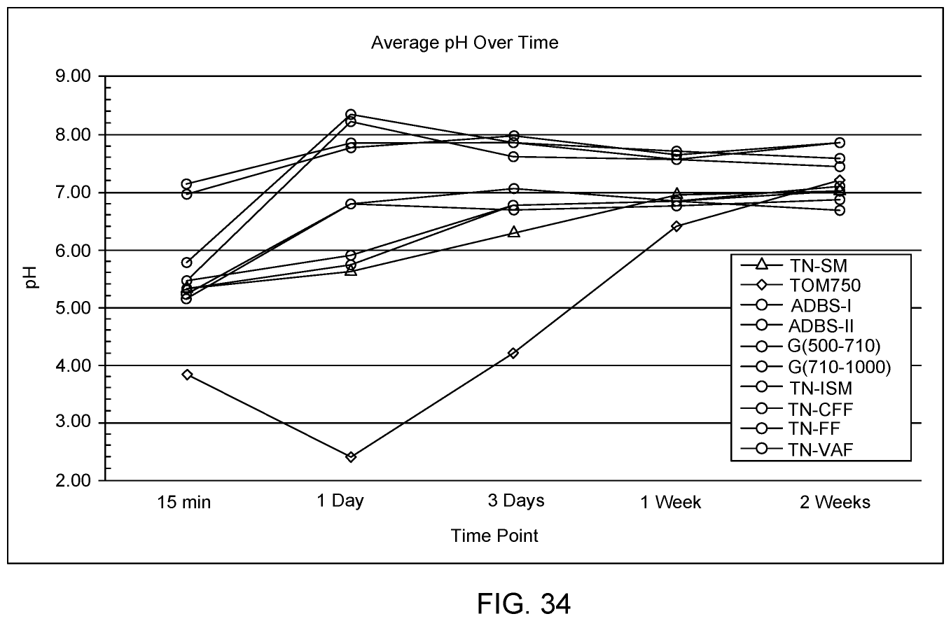

Measurements of acidity of the aqueous environment immediately surrounding the reaction mixture performed as pH measurements of the elution medium demonstrated the modulation of the observed pH drop. In this study, all formulations listed in Table 3 were formed into disks (approximately 10 mm diameter×5 mm height) and placed in a 0.001 M phosphate-buffered saline (PBS) solution in a ratio of 1 g of the composition to 5 mL of the 0.001 M PBS. Samples were prepared using a polyethylene mold and placed into the PBS solution at 2 minutes following product activation. The samples were removed at 15 minutes and punched out of the mold before being replaced in the solution for the remainder of the incubation time. Samples were incubated in a shaking water bath at 37° C. and 50 RPM for up to 2 weeks; specific analyzed time points included 15 minutes of incubation, 1 day of incubation, 3 days of incubation, 1 week of incubation, and 2 weeks of incubation. PBS media changes were performed daily, and pH measurements were taken at each time point for n=3 samples. The average of the pH measurements taken are illustrated in FIG. 34 and presented in Table 24 below. It should be noted that that the pH of elution media from all formulations universally changes with time from more acidic to more basic. It should further be noted that increased OPLS content, TOM750, generates more acidic elution media than lower OPLS content, TOM250, all else kept constant.

TABLE 28
Average pH of the Elution Medium Measured for Various Adhesive Compositions Over a Two-Week Period

| | Compo- | Compo- | | | | | | | | |
|---|---|---|---|---|---|---|---|---|---|---|
| | sition | sition | Composition | Composition | Composition | Composition | Composition | Composition | Composition | Composition |
| | 14 | 32 | 33 | 34 | 35 Granule | 36 Granule | 15 | 16 | 37 | 39 |
| Time Point | TN-SM | TOM 750 | ADBS-I | ADBS-II | (500-710) | (710-1000) | TN-ISM | TN-CFF | TN-FF | TN-VAF |
| 15 Min | 5.32 | 3.84 | 5.78 | 5.46 | 7.14 | 6.96 | 5.17 | 5.32 | 5.14 | 5.43 |
| 1 Day | 5.64 | 2.40 | 8.33 | 8.21 | 7.83 | 7.77 | 6.77 | 5.73 | 6.81 | 5.90 |
| 3 Days | 6.30 | 4.21 | 7.87 | 7.60 | 7.96 | 7.92 | 7.06 | 6.73 | 6.69 | 6.78 |
| 1 Week | 6.95 | 6.42 | 7.66 | 7.58 | 7.69 | 7.62 | 6.83 | 6.88 | 6.78 | 6.86 |
| 2 Weeks | 6.98 | 7.19 | 7.57 | 7.44 | 7.86 | 7.85 | 6.69 | 7.00 | 6.87 | 7.10 |

### Example 12: Influence of Methods on Functional Shear Bond Strength of the Composition When Cured

Functional shear bond strength of the cured adhesive compositions was assessed on benchtop by examining resistance to reverse torque of screw form dental implants (3.7 mm diameter×8 nun length) placed into prepared sites into 15 PCF solid foam (SAWBONES®) material. Initially, the implants were deemed not stable as they could be removed from the solid foam material by application of counterclockwise torque of 5 N-cm. The implants were stabilized and tested at 15 minutes post-insertion and application of the adhesive composition. The testing compared the implant stabilization strength using the non-pH-adjusted, dense adhesive composition (TN-SM) to the acidity adjusted and porous adhesive composition (TN-ISM). The maximum counterclockwise (reversal) torque levels required to break the bond and rotate the implants for the two adhesive compositions are presented in Table 25. The results showed a significant increase in the resistance to torque from the initial removal torque without the adhesive composition indicating an improved shear bond strength resulting from application of both compositions disclosed herein.

In another experiment involving the same two formulations, the same site geometry, the same implants, but conducted in vivo, the results of reverse torque application to failure demonstrate that stabilization by the porous, acidity adjusted formulation (TN-ISM) leads to greater resistance to removal torque than that of stabilization by the legacy non-acidity adjusted, relatively non-porous formulation (TN-SM), Table 25, right column. This functional difference in the level of stabilization provided by the two formulations has been shown to be related to bone ingrowth and osteointegration of the implants by host tissues.

TABLE 25
Comparison of reverse torque to failure values for dense and
porous formulation variants TN-SM and TN-ISM, respectively,
at t = 15 min (benchtop) and at six weeks (in vivo)
post-placement using Ø3.7 × 8 mm Tapered Screw Vent Implant

| | | | Maximum | Maximum |
|---|---|---|---|---|
| | | Implant | Torque at | Torque at |
| | Porosity | dimensions | t = 15 min | t = 6 wks |
| Formulation | (%) | D × L (mm) | (N-cm) | (N-cm) |
| TN-SM | 2.67 | Ø3.7 × 8 mm | 32.4 ± 4.8 | 18.1 ± 5.1 |
| TN-ISM | 29.9 | Ø3.7 × 8 mm | 17.1 ± 2.2 | 23.1 ± 7.2 |

Additionally, bone-bone adhesive shear strength was evaluated by gluing together, by their circular faces, two cylindrical bovine cortical bone blocks using the adhesive compositions. The testing was conducted to compare the adhesion strength of the bond formed using the TN-CFF adhesive composition. In one set of tests, the TN-CFF powders were mixed with either water as the aqueous medium or with the pH-adjusted aqueous medium using 0.8 M sodium hydroxide. The bone samples were pressed together immediately after applying the adhesive compositions to the bones after they were mixed. The samples were allowed to cure through incubation in PBS solution at 37° C. The mechanical testing was conducted at various curing times of the adhesive compositions. The shear testing of the bond between the bone blocks was conducted at 2 mm/min utilizing an Instron load frame machine.

TABLE 26
Adhesive Shear Stress (MPa) Required to Break the
Bond Between Bony Faces of Cortical Bone Blocks

| | | TN-CFF | TN-CFF |
|---|---|---|---|
| | | (with water) | (with NaOH) |
| 15 | minute (MPa) | 1.05 ± 0.2 | 0.93 ± 0.3 |
| 1 | hour (MPa) | 1.71 ± 0.5 | 1.95 ± 0.5 |
| 24 | hours (MPa) | 1.61 ± 0.5 | 2.08 ± 0.3 |

### Example 13: Influence of the Invention Methods on Phosphoserine (OPLS) Release

As discussed herein, OPLS is released from the adhesive compositions gradually throughout the initial curing period, followed by the gradual dissolution and resorption period of the adhesive composition matrix and is dependent on the available surface area of the adhesive composition that is in contact with the physiological fluids. During the setting of the adhesive composition, a percentage of OPLS is eluted from the cured adhesive product followed by the release of OPLS initial dissolution phase. This process was explored through in vitro incubation studies, presented here, that assessed the elution of OPLS from the various adhesive compositions over a two-week period.

Study 1: During this study, the elution profile of OPLS was assessed for TN-VAF, which is a low porosity composition, and for TN-ADBS, which is a hybrid acidity adjusted formulation composed of pre-cured granules embedded in a porous binder. In this study, the adhesive compositions were mixed and formed into cylindrical plugs (6 mm diameter×12 mm height) which weighed ˜0.675 g. The cylinders were incubated such that the mass of the plug to liquid medium ratio was 1 g to 50 mL 1× PBS solution at 37° C. for up to two weeks. The experimental conditions were designed to represent ample sink conditions, so that the solution would not reach saturation from OPLS released into the elution medium.

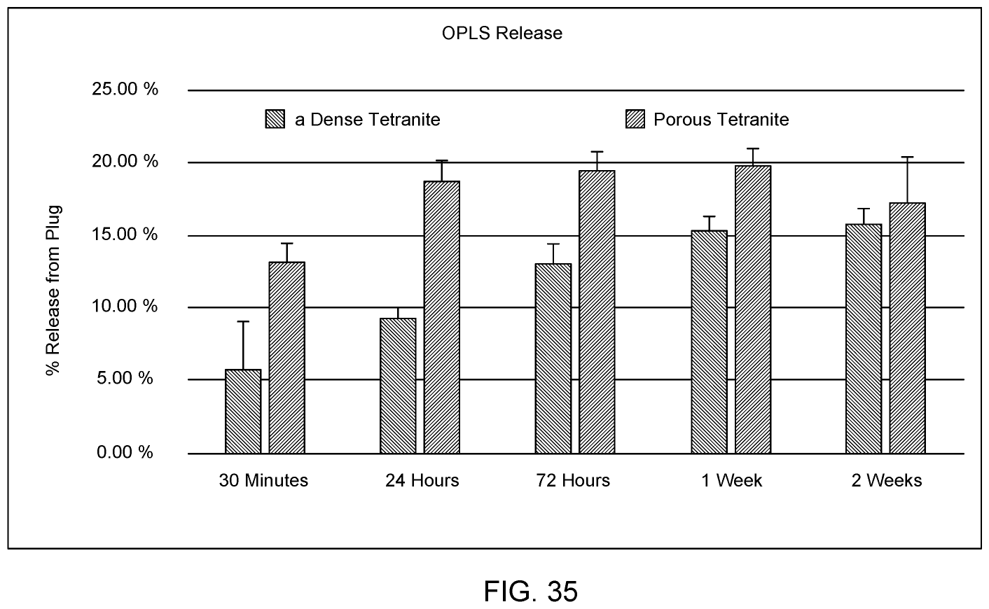

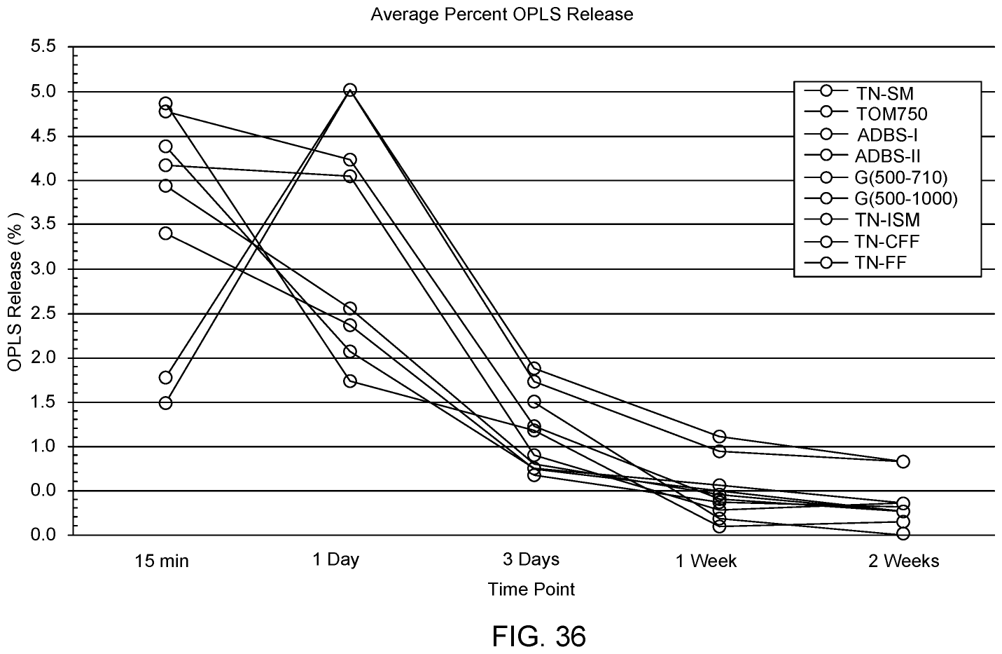

FIGS. 35 and 36 show the accumulated concentration and the percentage of the OPLS eluted out from samples over 2 weeks. The results show that 9% w/w of OPLS was released from the dense TN-VAF during its full setting reaction through Day 1. The release of OPLS was 18% w/w, or twice the amount, during this same period for TN-ADBS composition, which is characterized by open and interconnected porosity. Thereafter, the initial release of OPLS was governed by the available surface area of the composition. Hence, from a formed cylinder of dense TN-VAF (0.675 g), the OPLS eluted was 0.034 g (15.866% w/w of 0.215 g of OPLS) at the 2-week time point. Therefore, the concentration of OPLS in 50 mL solution was determined to be 0.69 mg/mL in the closed system. This increase in OPLS concentration may result in local biological response in vivo based upon previous work in which it was demonstrated that cellular response to OPLS is concentration dependent. Specifically, the OPLS induced amplification of osteoblast specific gene expression as reflected in the amount of mRNA only if its concentration is above 0.5 mg/mL threshold, concentrations less than 0.5 mg/mL yielding a similar gene expression to the controls.

Study 2: In this study, all formulations listed in Table 1 were formed into disks (approximately 10 mm diameter×5 mm height) and were placed in 0.001M PBS solution in a ratio of 1 g adhesive composition to 5 mL of 0.001M PBS. Samples were formed using a polyethylene mold and the adhesive composition was placed into the PBS solution by 2 minutes post product activation. The samples were removed at 15 minutes and punched out of the mold before being replaced in the solution for the remainder of the incubation time. Samples were incubated in a shaking water bath at 37° C. and 50 RPM for up to 2 weeks. PBS media changes were performed daily to represent an open system. FIG. 36 illustrates a comparison of the percentage of OPLS released for each formulation. Table 27 below shows the total percentage of OPLS released in addition to the OPLS released per time interval. Formulations utilizing porous granules were shown to have the highest total percentage of OPLS released over the 2-week elution period.

TABLE 27
Percentage of OPLS Released from Various Adhesive Compositions
Over 2-week Incubation Period, (n = 3 samples each)

| | TN- | TOM | ADBS- | ADBS- | Granule | Granule | TN- | TN- | TN- | TN- |
|---|---|---|---|---|---|---|---|---|---|---|
| Time | SM | 750 | I | II | (500-710) | (500-1000) | ISM | CFF | FF | VAF |
| 15 Min | 4.88 | N/A | 4.18 | 4.78 | 1.49* | 1.78 | N/A | 3.95 | 4.39 | 3.42 |
| 1 Day | 1.75 | N/A | 4.05 | 4.24 | 5.02 | 5.02 | N/A | 2.56 | 2.08 | 2.38 |
| 3 Days | 1.19 | 1.52 | 0.92 | 1.24 | 1.73 | 1.89 | 0.69 | 0.81 | 0.75 | 0.77 |
| 1 Week | 0.11 | 0.18 | 0.31 | 0.42 | 0.95 | 1.13 | 0.37 | 0.48 | 0.58 | 0.55 |
| 2 Weeks | 0.15 | 0.03 | 0.30 | 0.29 | 0.82 | 0.85 | 0.34 | 0.27 | 0.36 | 0.26 |
| Total % | 8.08 | 1.73 | 9.76 | 10.97 | 10.01 | 10.67 | 1.40 | 8.07 | 8.15 | 7.37 |
| OPLS | | | | | | | | | | |

### Example 14: Influence of the Method on Biocompatibility In Vitro I

In vitro cytotoxicity assessments were performed per ISO 10993-5 using L-929 mouse fibroblast cells. This testing, the results of which are listed in Table 28 demonstrated a significant improvement in cell viability when the pH-adjusted adhesive compositions from Table 3 (TN-ISM, TN-CFF) are compared to the non-pH-adjusted, dense adhesive composition (TN-SM).

TABLE 28
Cytotoxicity of the Adhesive Compositions

| Formulation | Score |
|---|---|
| TN-SM | Mild (2) - not more than 50% of the cells are round, devoid |
| | of intracytoplasmic granules; no extensive cell lysis; not |
| | more than 50% growth inhibition observable |
| TN-ISM | None (0) - discrete intracytoplasmic granules, no cell |
| | lysis, no reduction of cell growth |
| TN-CFF | None (0) - discrete intracytoplasmic granules, no cell lysis, |
| | no reduction of cell growth |

### Example 15. Influence of Methods of this Invention on the Rate of Replacement of the Adhesive Composition by New Bone In Vivo A

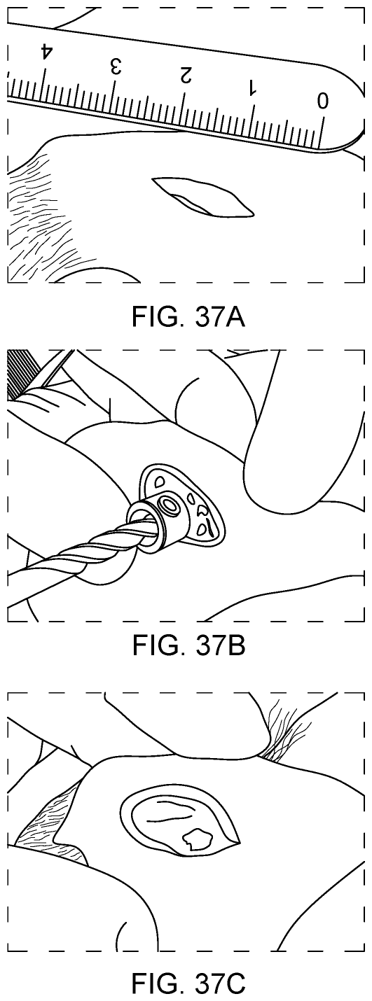

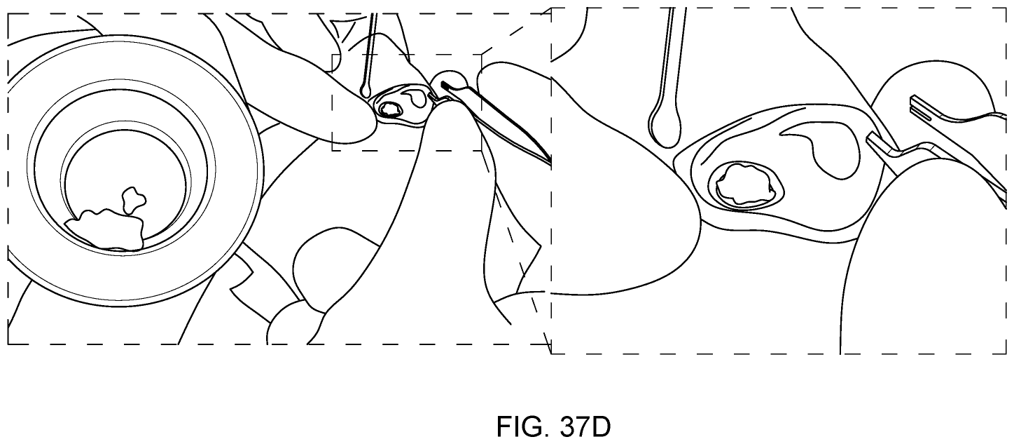

An experiment was designed to compare biological response of the non-pH-adjusted, dense adhesive composition (TN-SM) to the pH-adjusted and porous adhesive composition (TN-ISM) in the New Zealand White rabbit distal femur critical size defect model as illustrated in FIGS. 37A-37D. The model consisted of a 5 mm diameter by 10 mm long bony defect in the lateral condyle at the region of the epiphyseal plate of a skeletally mature animal. The defect of this size is a critical size, i.e., it does not fill with hone spontaneously. Biomaterials which are implanted into the defect can be compared as to both their regenerative potential, i.e., new bone formation, and to induction of adverse responses, e.g., excessive inflammation.

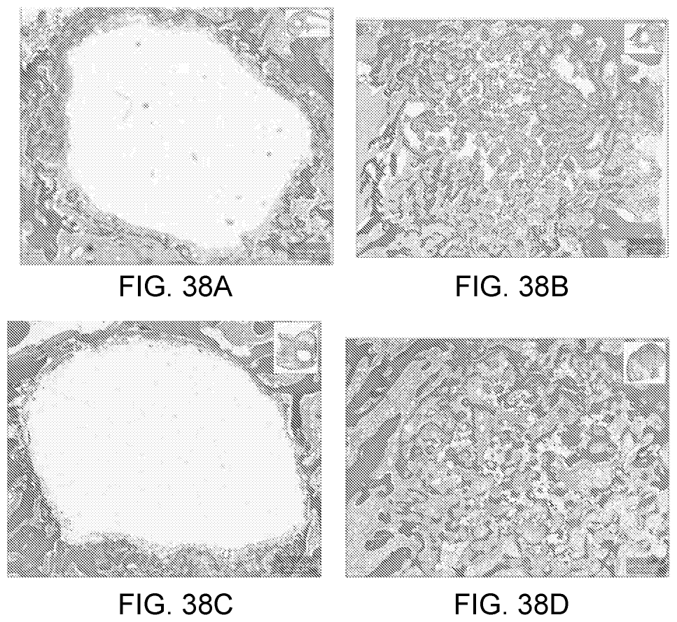

The results of implantation of the non-pH-adjusted adhesive composition (TN-SM) to the pH-adjusted, porous material (TN-ISM) demonstrated a clear difference in the regenerative potential of the two test compositions. Neither adhesive composition produced an adverse response. FIGS. 38A-38D, showing photomicrographs of the implantation sites at 3 weeks and 8 weeks post-implantation, demonstrated the result of accelerated regenerative process at the sites where the TN-ISM adhesive composition was placed.

### Example 16. Influence of the Method of this Invention the Rate of Replacement of the Adhesive Composition by New Bone In Vivo B

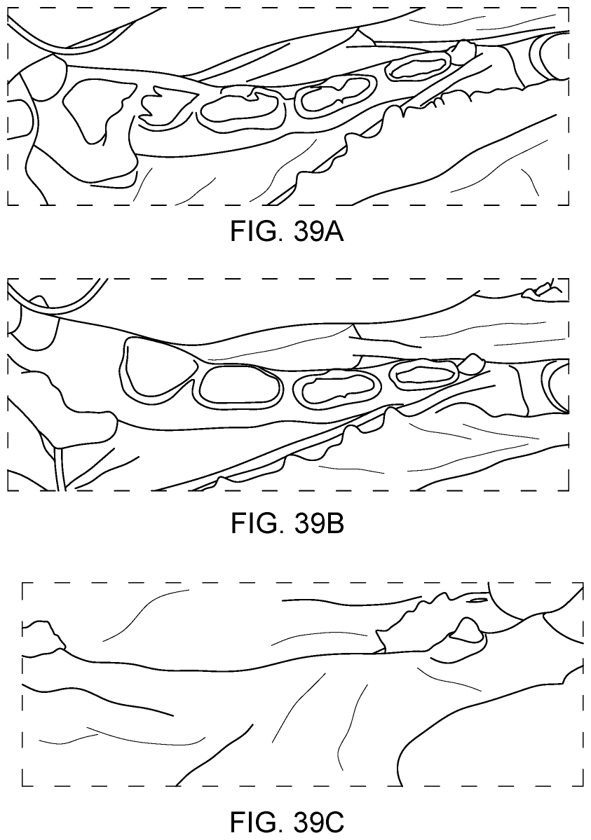

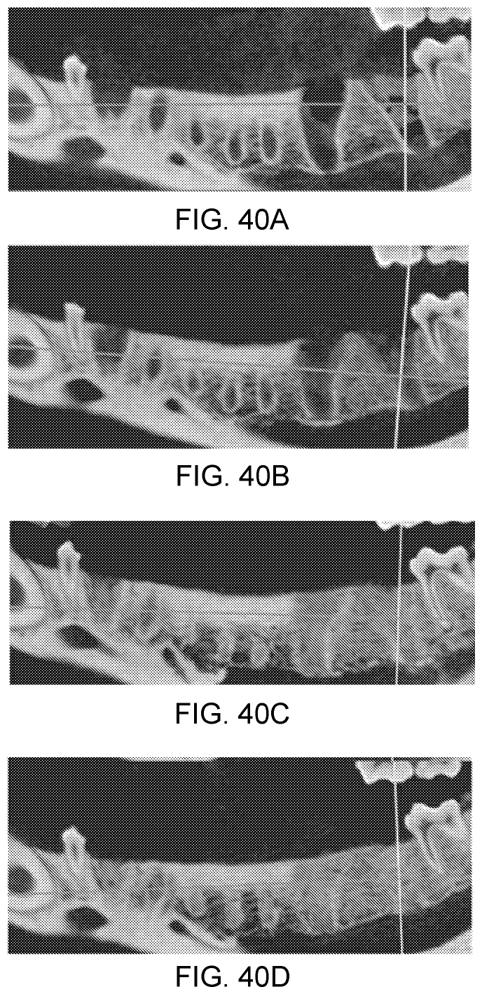

In this study, the rapid turnover of the pH-adjusted and porous composition (TN-ADBS) was explored by implanting the TN-ADBS composition in a precured, granulated form into a bony defect in a canine mandibular model as illustrated in FIGS. 39A-39C. FIGS. 40A-40D illustrate the accelerated dissolution-resorption of the radiopaque granules in CT images in sagittal and coronal planes. The radiographic reconstructions demonstrated the immediate post-implantation (FIG. 40A), two week (FIG. 40B), four week (FIG. 40C), and six week (FIG. 40C) post-implantation states of the site. At the two week time point as illustrated in FIG. 40B, the radiopacity associated with the granules is no longer evident at the site. The increased radiodensity evident at weeks 4 and 6, shown in FIGS. 40C and 40D, respectively, was associated with the deposition of new trabecular and cortical bone generated at the defect site and replacement of the implanted biomaterial.

### Example 17. Effects of the Addition of Citric Acid and Calcium Citrate

In this example, Compositions 39-42 as listed in Table 3 were prepared with varying particle sizes of the multivalent metal salt to investigate its effects on the porosity, pH, and functional characteristics such as adhesive shear. These compositions also included pH adjusting modalities such as citric acid and calcium citrate.

### Method

The compositions were mixed for 30 seconds, after which the mixture was formed into a 6 mm diameter and 12 mm high cylinder and allowed to cure for 15-20 minutes. The substrate was then punched out and incubated in 0.001M PBS in a ratio of 1 g/5 mL liquid. After curing, the pH of the elution medium was tested, and porosity and compression of the resultant compositions were evaluated as shown in Table 29 below.

TABLE 29
Test Description, sample size and time points

| Test Description | Sample Size | Timepoints |
|---|---|---|
| Elution - pH | 3/Timepoint | T = 30 min, 1 hr, 2 hr, |
| | | 6 hr, 24 hr, 48 hr |
| Density and Porosity | 3/Timepoint | T = 40-48 hr |
| Compression | 3/Timepoint | T = 40-48 hr |
| Adhesive Shear | 3/Timepoint | T = 40-48 hours |

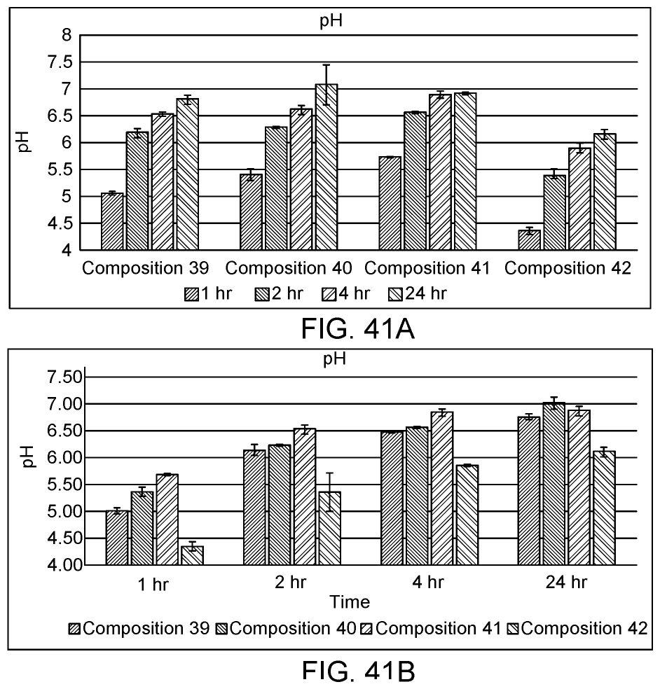

The detailed methods are described in Example 1. The compositions were tested to determine its adhesive shear. The results are presented in Table 30. The pH of the elution medium for each of Compositions 39-42 is illustrated in FIGS. 41A-41B. The data showed that the mechanical properties of the compositions such as the injection force and the adhesive shear were not compromised when the particle size of multivalent metal salt was 250 μm.

TABLE 30
Results of Density, Porosity and Compression

| | | | | Pore size | Pore Surface | | |
|---|---|---|---|---|---|---|---|
| | | | Pore size | percentiles by | Area to Total | | |
| | | | percentiles by | cumulative | Material | | |
| | | Porosity | count (μm) | volume (μm) | Volume | Compressive | Adhesive |
| | Density | (%) | (d10, d50, d90) | (d10, d50, d90) | Ratio (mm⁻¹) | Stress | Shear |
| Composition | (g/cc) | Mean ± s.d. | Mean ± s.d. | Mean ± s.d. | Mean ± s.d | (MPa) | (MPa) |
| Composition | 1.98 | 4.4 ± .2 | 19 ± 0, | 289 ± 44, | .54 ± .06 | 35.12 | 3.23 |
| 39 | | | 19 ± 0, | 1187 ± 24, | | | |
| | | | 43 ± 2 | 1187 ± 24 | | | |
| Composition | 1.26 | 26.3 ± 4.3 | 19 ± 0, | 661 ± 53, | 9.71 ± .11 | 5.78 | 0.87 |
| 40 | | | 23 ± 0, | 2199 ± 147, | | | |
| | | | 61 ± 7 | 2199 ± 147 | | | |
| Composition | 0.95 | 52.3 ± .3 | 19 ± 0, | 406 ± 9, | 11.03 ± .72 | 2.49 | 0.19 |
| 41 | | | 19 ± 0, | 3184 ± 17, | | | |
| | | | 36 ± 2 | 3184 ± 17 | | | |
| Composition | 1.96 | 7.1 ± .2 | 19 ± 0, | 310 ± 9, | 1.19 ± .20 | 13.6 | 2.75 |
| 42 | | | 19 ± 0, | 1664 ± 7, | | | |
| | | | 54 ± 2 | 1664 ± 7 | | | |

### Example 18. Effects of the Particle Size of the Multivalent Metal Salt and Calcium Carbonate on High Porosity Compositions

In this example, Compositions 32, 43, and 44 as recited in Table 3 were prepared with varying particle sizes of the multivalent metal salt to investigate its effects on the porosity and pH of a high porous composition containing 20% w/w calcium carbonate.

### Method

The compositions were mixed for 30 seconds, after which the mixture was formed into a puck and allowed to cure for 15-20 minutes. The substrate was then punched out and incubated in 0.001 M phosphate buffered saline in a ratio of 1 g/5 mL liquid. After curing, the pH of the elution medium was tested, and porosity and compression of the resultant compositions were evaluated as shown in Table 31 below.

TABLE 31
Test Description, sample size and time points

| Test Description | Sample Size | Timepoints |
|---|---|---|
| Elution - pH | 3/Timepoint | T = 30 min, 1 hr, 2 hr, |
| | | 6 hr, 24 hr, 48 hr |
| Porosity | 3/Timepoint | T = 40-48 hr |

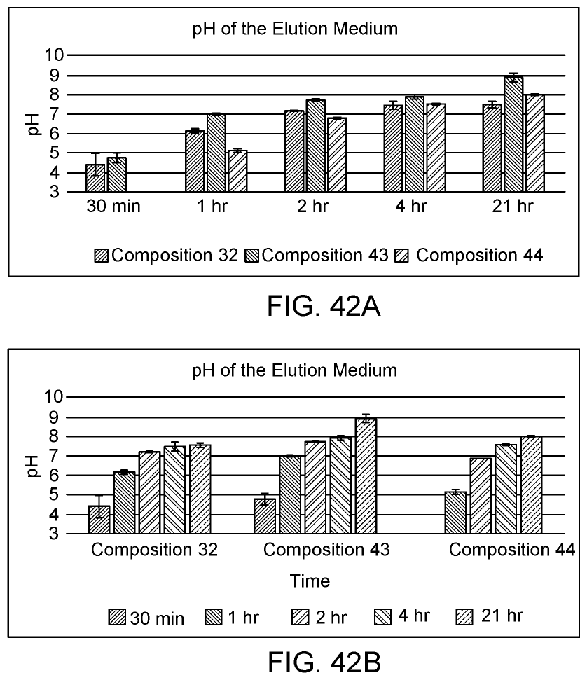

The detailed methods are described in Example 1. The compositions were tested to determine their porosity, pore size percentile by count, and pore size percentiles by cumulative volume. The results are presented in Table 32. The pH of the elution medium for each of Compositions 32, 43, and 44 is illustrated in FIGS. 42A-42B. The data showed that up to 76% of porosity using calcium carbonate as the porogen while maintaining the pH between 4.4 and 9.

TABLE 32
Results of Porosity

| | | | Pore size | |
|---|---|---|---|---|
| | | Pore size | percentiles | Pore Surface |
| | | percentiles | by cumulative | Area to Total |
| | | by count | volume | Material |
| | Porosity | (μm) (d10, | (μm) (d10, | Volume Ratio |
| Composition | (%) | d50, d90) | d50, d90) | (mm⁻¹) |
| Compo- | 76.9 | 19, 23, 45 | 194, 3619, 3619 | 5.68 |
| sition 32 | | | | |
| Compo- | 76.1 | 19, 19, 36 | 122, 2962, 2962 | 6.36 |
| sition 43 | | | | |
| Compo- | 61.5 | 19, 23, 64 | 632, 3366, 3366 | 5.21 |
| sition 44 | | | | |

### Example 19. Porous Adhesive Composition Using α-Tricalcium Phosphate and Calcium Silicate as the Multivalent Metal Salt

In this example, Compositions 45-48 as listed in Table 3 were prepared using a novel multivalent metal salt system including α-tricalcium phosphate and calcium silicate.

### Method

The compositions were mixed for 30 seconds, after which the mixture was formed into a 6 mm diameter and 12 mm high cylinder and allowed to cure for 15-20 minutes. The substrates were then punched out and incubated in 0.001 M phosphate buffered saline in a ratio of 1 g15 mL liquid. After curing, the substrates were tested for porosity adhesive sheer, and compression. The pH of the elution medium was measured. The test parameters are listed in Table 33.

TABLE 33
Summary of the test plan, sample size and time points

| Test Description | Sample Size | Timepoints |
|---|---|---|
| Elution - pH | 3/Timepoint | T = 30 min, 1 hr, 2 hr, |
| | | 6 hr, 24 hr, 48 hr |
| Density and Porosity | 3/Timepoint | T = 40-48 hr |
| Compression | 3/Timepoint | T = 40-48 hr |
| Adhesive Shear | 3/Timepoint | T = 40-48 hours |

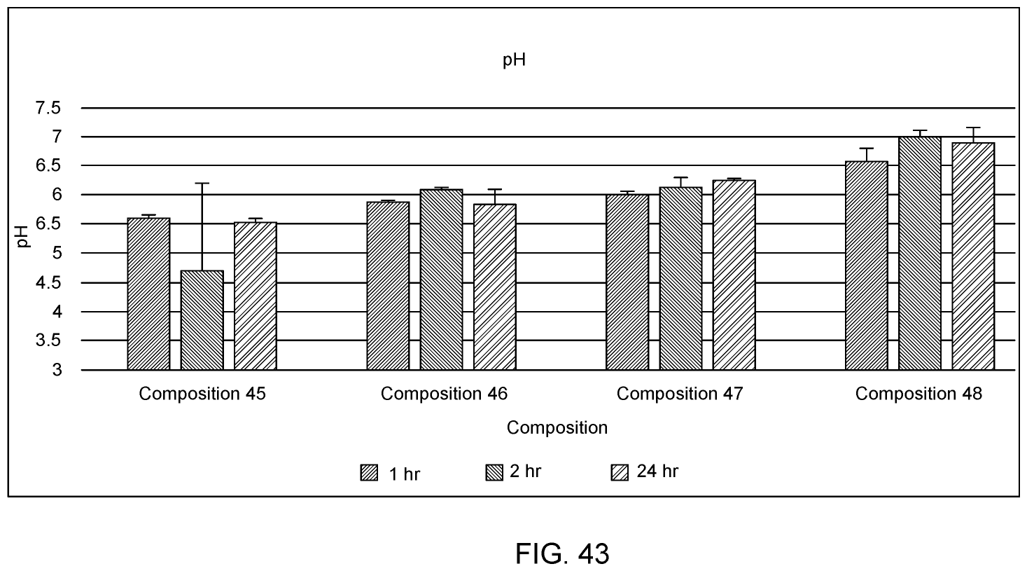

The results are shown in Table 33 and FIG. 43. The data confirmed that each of Compositions 45-48 had similar adhesive shear while the compressive stress decreased with the addition of calcium carbonate. The pH of the elution medium is shown in FIG. 43.

TABLE 34
Results (mean) showing Density, Porosity and Compression

| | | Compressive | Adhesive |
|---|---|---|---|
| | Density | Stress (MPa) | Shear (MPa) |
| Composition | (g/cc) | (n = 1) | (n = 1) |
| Composition 45 | 1.47 | 11.54 | 1.35 |
| Composition 46 | 1.6 | 9.9 | 1.31 |
| Composition 47 | 1.24 | 1.9 | 1.0 |
| Composition 48 | 1.26 | 2.87 | 0.8 |

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

What is claimed is:

1. An adhesive composition comprising:
i) a multivalent metal salt;
ii) an multidentate acidic organic compound;
iii) a porogen; and
iv) an aqueous medium,
wherein the adhesive composition, upon curing, has a porosity between about 10% to about 50%.

2. The adhesive composition of claim 1, wherein the porogen comprises a calcium salt (e.g., calcium carbonate).

3. The adhesive composition of claim 1, wherein the adhesive composition, upon curing, comprises a plurality of pores.

4. The adhesive composition of claim 1, wherein upon curing, the composition comprises a plurality of pores having a pore size of between 20 μm to 200 km.

5. The adhesive composition of claim 1, wherein the adhesive composition has a density of about 0.75 to about 1.40 g/cm³.

6. The adhesive composition of claim 1, wherein the pH of the milieu surrounding the adhesive composition is less than 8.5.

7. The adhesive composition of claim 1, wherein the pH of the adhesive composition in a tacky state is between 7 to 10 (e.g., greater than 6.5, wherein X=5, 6, 7, 8, or 9).

8. The adhesive composition of claim 1, wherein the multivalent metal salt comprises calcium phosphate, e.g., tetracalcium phosphate or tricalcium phosphate, e.g., α-tricalcium phosphate or β-tricalcium phosphate, calcium citrate, calcium carbonate, magnesium phosphate, sodium silicate, lithium phosphate, titanium phosphate, strontium phosphate, zinc phosphate, calcium oxide, magnesium oxide, or a combination thereof.

9. The adhesive composition of claim 1, wherein the multidentate acidic organic compound is a compound of Formula (I) or a salt thereof: 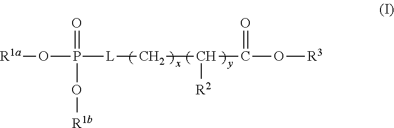
wherein:
L is O, S, NH, or CH₂;
each of R^{1a} and R^{1b} is independently H, optionally substituted alkyl, or optionally substituted aryl;
R² is H, NR^{4a}R^{4b}, C(O)R⁵, or C(O)OR⁵;
R³ is H, optionally substituted alkyl, or optionally substituted aryl;
each of R^{4a} and R^{4b} is independently H, C(O)R⁶, or optionally substituted alkyl;
R⁵ is H, optionally substituted alkyl, or optionally substituted aryl;
R⁶ is optionally substituted alkyl or optionally substituted aryl; and
each of x and y is independently 0, 1, 2, or 3.

10. The adhesive composition of claim 9, wherein the multidentate acidic organic compound comprises phosphoserine.

11. The adhesive composition of claim 1, wherein the multidentate acidic organic compound is present within the adhesive composition in an amount between 10% and 90% (w/w) of the total weight.

12. The adhesive composition of claim 1, wherein the aqueous medium comprises water, saliva, saline, serum, plasma, or blood.

13. The adhesive composition of claim 1, wherein the adhesive composition further comprises an additive.

14. The adhesive composition of claim 13, wherein the additive comprises a salt, filler, formulation base, viscosity modifier, abrasive, coloring agent, flavoring agent, or polymer.

15. The adhesive composition of claim 14, wherein the polymer comprises poly(L-lactide), poly(D,L-lactide), polyglycolide, poly(ε-caprolactone), poly(teramethylglycolic-acid), poly(dioxanone), poly(hydroxybutyrate), poly(hydroxyvalerate), poly(lactide-co-glycolide), poly(glycolide-co-trimethylene carbonate), poly(glycolide-co-caprolactone), poly(glycolide-co-dioxanone-co-trimethylene-carbonate), poly(tetramethylglycolic-acid-co-dioxanone-co-trimethylenecarbonate), poly(glycolide-co-caprolactone-co-lactide-co-trimethylene-carbonate), poly(hydroxybutyrate-co-hydroxyvalerate), poly(methylmethacrylate), poly(acrylate), polyamines, polyamides, polyimidazoles, poly(vinyl-pyrrolidone), collagen, silk, chitosan, hyaluronic acid, gelatin, or a mixture thereof.

16. The adhesive composition of claim 1, wherein the adhesive composition during the tacky state has a tack stress of between about 10 kPa and about 250 kPa after mixing with the aqueous medium.

17. The adhesive composition of claim 1, wherein the multivalent salt, the multidentate acidic organic compound, and/or the porogen is provided as a powder.

18. The adhesive composition of claim 17, wherein the mean particle size of the powder is about 0.0001 to about 1.000 mm, about 0.0005 to about 0.001 mm, about 0.001 to about 0.025 mm, about 0.005 to about 0.015 mm, about 0.001 to about 0.250 mm, about 0.005 to about 0.150 mm, about 0.250 to about 0.750 mm, about 0.25 to about 0.50 mm, about 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm.

19. The adhesive composition of any one of claims 3-4, wherein the pores range in size from 0.01 mm to 1.0 mm.

20. The adhesive composition of claim 1, where in the aqueous medium further comprises a pH adjusting agent comprising sodium hydroxide.

21. A method of preparing an adhesive composition, comprising:
(i) providing a mixture of a multivalent metal salt, multidentate acidic organic compound, and a carbonate salt;
(ii) contacting the mixture with an aqueous medium; and
(iii) adding a pH adjusting agent to the mixture to bring a pH of the mixture to a value between 7 to 10,
thereby preparing the adhesive composition.

22. A method of preparing an adhesive composition, comprising:
(i) providing a mixture of a multivalent metal salt, multidentate acidic organic compound, and a carbonate salt;
(ii) contacting the mixture with an aqueous medium; and
(iii) adding a pH adjusting agent to the mixture to evolve a gas from the mixture,
wherein the adhesive composition, upon curing, has a porosity between about 10% to about 50%,
thereby preparing the adhesive composition.

23. A method of preparing an adhesive composition, comprising:
(i) providing a mixture of a multivalent metal salt, multidentate acidic organic compound, and a carbonate salt;
(ii) contacting the mixture with an aqueous medium; and
(iii) adding a pH adjusting agent to the mixture to evolve a gas from the mixture,
wherein the adhesive composition, upon curing, has a plurality of pores having a pore size of between 20 μm to 200 m,
thereby preparing the adhesive composition.

24. The method of any one of claims 21-23, wherein the pH adjusting agent is a basic pH adjusting agent.

25. The method of claim 24, wherein the basic pH adjusting agent comprises an alkali metal hydroxide, an alkali metal oxide, an alkaline earth metal hydroxide, an alkaline earth metal oxide, or a combination thereof.

26. The method of any one of claims 24-25, wherein the basic pH adjusting agent comprises a basic pH adjusting agent selected from the group consisting of sodium hydroxide, potassium hydroxide, lithium hydroxide, calcium hydroxide, magnesium hydroxide, or a combination thereof.

27. The method of any one of claims 24-26, wherein the basic pH adjusting agent comprises sodium hydroxide.

28. A method of generating or regenerating bone tissue, the method comprising:
a) preparing an adhesive composition comprising a multivalent metal salt, an multidentate acidic organic compound, and a carbonate salt in an aqueous medium;
b) applying the adhesive composition to a site (e.g., into or onto bone, or in between bones or bone fragments); and
c) allowing the adhesive composition to harden, cure, or be resorbed by bone, the adhesive composition, upon curing, having a porosity between about 10% to about 50%;
thereby generating or regenerating bone tissue.

29. A method of inducing osteoblast formation, the method comprising:
a) preparing an adhesive composition comprising a multivalent metal salt, an multidentate acidic organic compound, and a carbonate salt in an aqueous medium;
b) applying the adhesive composition to a site (e.g., into or onto bone, or in between bones or bone fragments); and
c) allowing the adhesive composition to harden, cure, or be resorbed by bone, the adhesive composition, upon curing, having a porosity between about 10% to about 50%;
thereby inducing osteoblast formation.

30. A method of treating or preventing a bone disease or disorder in a subject, the method comprising:
a) preparing an adhesive composition comprising a multivalent metal salt, an multidentate acidic organic compound, and a carbonate salt in an aqueous medium;
b) applying the adhesive composition to a site (e.g., into or onto bone, or in between bones or bone fragments); and
c) allowing the adhesive composition to harden, cure, or be resorbed by bone, the adhesive composition, upon curing, having a porosity between about 10% to about 50%;
thereby treating or preventing the bone disease or disorder in the subject.

31. A method of inducing expression of MKI67, the method comprising:
a) preparing an adhesive composition comprising a multivalent metal salt, an multidentate acidic organic compound, and a carbonate salt in an aqueous medium;
b) applying the adhesive composition to a site (e.g., into or onto bone, or in between bones or bone fragments); and
c) allowing the adhesive composition to harden, cure, or be resorbed by bone, the adhesive composition, upon curing, having a porosity between about 10% to about 50%,
thereby inducing expression of MKI67.

32. A method of inducing expression of Bone Morphogenetic protein-2 (BMP2), the method comprising:
a) preparing an adhesive composition comprising a multivalent metal salt, an multidentate acidic organic compound, and a carbonate salt in an aqueous medium;
b) applying the adhesive composition to a site (e.g., into or onto bone, or in between bones or bone fragments); and
c) allowing the adhesive composition to harden, cure, or be resorbed by bone, the adhesive composition, upon curing, having a porosity between about 10% to about 50%;
thereby inducing expression of Bone Morphogenetic protein-2 (BMP2).

33. A method of inducing expression of Vascular Endothelial Growth factor (VEGF), the method comprising:
a) preparing an adhesive composition comprising a multivalent metal salt, an multidentate acidic organic compound, and a carbonate salt in an aqueous medium;
b) applying the adhesive composition to a site (e.g., into or onto bone, or in between bones or bone fragments); and
c) allowing the adhesive composition to harden, cure, or be resorbed by bone, the adhesive composition, upon curing, having a porosity between about 10% to about 50%;
thereby inducing expression of Vascular Endothelial Growth factor (VEGF).

34. A method of inducing expression of one or both of caspase-3 and caspase-9, the method comprising:
a) preparing an adhesive composition comprising a multivalent metal salt, an multidentate acidic organic compound, and a carbonate salt in an aqueous medium;
b) applying the adhesive composition to a site (e.g., into or onto bone, or in between bones or bone fragments); and
c) allowing the adhesive composition to harden, cure, or be resorbed by bone, the adhesive composition, upon curing, having a porosity between about 10% to about 50%;
thereby inducing expression of one or both of caspase-3 and caspase-9.

35. A method of inducing expression of Interleukin-6 (IL-6), the method comprising:
a) preparing an adhesive composition comprising a multivalent metal salt, an multidentate acidic organic compound, and a carbonate salt in an aqueous medium;
b) applying the adhesive composition to a site (e.g., into or onto bone, or in between bones or bone fragments); and
c) allowing the adhesive composition to harden, cure, or be resorbed by bone, the adhesive composition, upon curing, having a porosity between about 10% to about 50%;
thereby inducing expression of Interleukin-6 (IL-6).

36. A method of inducing expression of tumor necrosis factor alpha (TNF-α), the method comprising:
a) preparing an adhesive composition comprising a multivalent metal salt, an multidentate acidic organic compound, and a carbonate salt in an aqueous medium;
b) applying the adhesive composition to a site (e.g., into or onto hone, or in between bones or bone fragments); and
c) allowing the adhesive composition to harden, cure, or be resorbed by bone, the adhesive composition, upon curing, having a porosity between about 10% to about 50%;
thereby inducing expression of tumor necrosis factor alpha (TNF-α).

37. The method of any one of claims 21-36, wherein the adhesive composition, upon curing, comprises a plurality of pores.

38. The method of any one of claims 21-36, wherein the adhesive composition is porous and upon curing has a plurality of pores having a pore size of between 20 m to 200 m.

39. The method of any one of claims 21-36, wherein the adhesive composition further comprises a pH adjusting agent.

40. The method of any one of claims 21-36, wherein the adhesive composition has a density of about 0.75 to about 1.40 g/cm³.

41. The method of any one of claims 21-36, wherein the pH of the milieu surrounding the adhesive composition is less than 8.5.

42. The method of any one of claims 21-36, wherein the pH of the adhesive composition in a tacky state is between 7 to 10 (e.g., greater than 6.5, wherein X=5, 6, 7, 8, or 9).

43. The method of any one of claims 21-36, wherein the multivalent metal salt comprises calcium phosphate, e.g., tetracalcium phosphate or tricalcium phosphate, e.g., α-tricalcium phosphate or β-tricalcium phosphate, calcium citrate, calcium carbonate, magnesium phosphate, sodium silicate, lithium phosphate, titanium phosphate, strontium phosphate, zinc phosphate, calcium oxide, magnesium oxide, or a combination thereof.

44. The method of any one of claims 21-36, wherein the multidentate acidic organic compound is a compound of Formula (I) or a salt thereof: 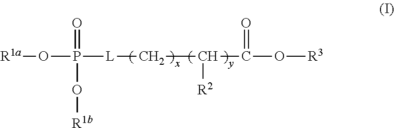
wherein:
L is O, S, NH, or CH₂;
each of R^{1a} and R^{1b} is independently H, optionally substituted alkyl, or optionally substituted aryl;
R² is H, NR^{4a}R^{4b}, C(O)R⁵, or C(O)OR⁵;
R³ is H, optionally substituted alkyl, or optionally substituted aryl;
each of R^{4a} and R^{4b} is independently H, C(O)R⁶, or optionally substituted alkyl;
R⁵ is H, optionally substituted alkyl, or optionally substituted aryl;
R⁶ is optionally substituted alkyl or optionally substituted aryl; and
each of x and y is independently 0, 1, 2, or 3.

45. The method of claim 44, wherein the multidentate acidic organic compound comprises phosphoserine.

46. The method of any one of claims 21-36, wherein the multidentate acidic organic compound is present within the adhesive composition in an amount between 10% and 90% (w/w) of the total weight.

47. The method of any one of claims 21-36, wherein the aqueous medium comprises water, saliva, saline, serum, plasma, or blood.

48. The method of any one of claims 21-36, wherein the adhesive composition further comprises an additive.

49. The method of claim 48, wherein the additive comprises a salt, filler, formulation base, viscosity modifier, abrasive, coloring agent, flavoring agent, or polymer.

50. The method of any one of claims 21-36, wherein the carbonate salt comprises calcium carbonate.

51. The method of any one of claims 21-36, wherein the adhesive composition during a tacky state has a tack stress of between about 10 kPa and about 250 kPa after mixing with the aqueous medium.

52. The method of any one of claims 21-36, wherein the multivalent metal compound is provided as a powder.

53. The method of claim 52, wherein a mean particle size of the powder is about 0.0001 to about 1.000 mm, about 0.0005 to about 0.001 mm, about 0.001 to about 0.025 mm, about 0.005 to about 0.015 mm, about 0.001 to about 0.250 mm, about 0.005 to about 0.150 mm, about 0.250 to about 0.750 mm, about 0.25 to about 0.50 mm, about 0.10 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm.

54. The method of any one of claims 21-36, wherein pores range in size from 0.01 mm to 1.0 mm.

55. The adhesive composition or method of any one of the proceeding claims, wherein the multivalent metal salt has at least 10% of its particle size below 45 μm.
